(19) United States
(12) Patent Application Publication
(10) Pub. No.: US 2009/0178161 A1
(43) Pub. Date: Jul. 9, 2009

(54) METHOD FOR DIRECTING NUCLEIC ACIDS TO PLASTIDS

(76) Inventors: Chantal Arar, Draveil (FR); Richard De Rose, Raleigh, NC (US); Anne Duprat, Noisy Le Sec (FR); Jacques Joyard, Meylan (FR); Maryse Nicolai, Brignoles (FR); Christophe Robaglia, Venelles (FR); Norbert Rolland, Saint-Egreve (FR); Daniel Salvi, Tullins (FR); Rodnay Sormani, Aix En Provence (FR)

Correspondence Address: STITES & HARBISON PLLC, 1199 NORTH FAIRFAX STREET, SUITE 900, ALEXANDRIA, VA, 22314, US

(21) Appl. No.: 11/720,133

(22) PCT Filed: Nov. 25, 2005
(86) PCT No.: PCT/FR05/02940

§ 371 Date: Mar. 3, 2008

(30) Foreign Application Priority Data
Nov. 26, 2004 (FR) 0412601

(51) Int. Cl. A01H 5/00; C12N 15/82; C12N 15/74; C12N 5/10; C12P 21/00

(52) U.S. Cl. 800/298; 435/468; 435/320.1; 435/410; 435/69.1

(57) ABSTRACT

The invention relates to nucleic acid sequences naturally imported into a plant cell plastid, and use thereof for directing an RNA sequence of interest to a plastid, which permits, in particular, the directed expression of a protein of interest in a plant cell plastid.

The invention relates to nucleic acid sequences naturally imported into a plant cell plastid, and to the use thereof for targeting an RNA sequence of interest to a plant cell plastid, thus allowing in particular the directed expression of a protein of interest in a plant cell plastid.

Over the last fifteen years or so, a concept has emerged according to which the subcellular localization of mRNA is thought to be a key mechanism for directing gene products to individual subcellular compartments, or to specific regions of a cell or of an embryo, thus constituting an important mechanism for post-transcriptional regulation of gene expression. This mRNA localization phenomenon occurs in all three of unicellular organisms, animals and plants. The mechanisms that may contribute to this subcellular localization of mRNA have been reviewed by Kloc et al. (2002).

The first pieces of evidence of a specific subcellular localization of mRNA in plant cells are very recent. Cellular polarization has thus been demonstrated in differentiating xylem cells in which an exclusive localization at the basal pole or the apical pole has been observed depending on the type of expansin mRNA considered (Im et al., 2000), expansins being cell wall proteins. mRNAs of storage proteins in rice have, moreover, been localized in specific subdomains of the endoplasmic reticulum (Choi et al., 2000).

Plastids are semiautonomous organelles which exhibit great structural diversity and contain unique biosynthetic pathways. In particular, chloroplasts are supposed to be derived from an endosymbiosis between a photosynthetic bacterium and a eukaryotic cell. The appropriate function of this association requires a high degree of integration between the chloroplast genome and the cellular genome of the plant. Many chloroplast genes have been transferred to the nucleus of the host cell and the proteins encoded by these genes are subsequently imported into the chloroplast (Martin and Herrmann, 1998; Joyard et al., 1998). The chloroplast activity also regulates the expression of these genes at the transcriptional and post-transcriptional level (Surpin et al., 2002; Petracek et al., 1997).

More generally, plastid function is highly dependent on the proteins which are encoded in the nucleus, translated in the cytoplasm and imported into the plastids. In fact, most genes encoding plastid proteins are nuclear and the proteins are therefore translocated to the plastids by means of protein importation machinery contained in the plastid envelope membrane. Surprisingly, the importation of RNA molecules from the nucleus or the cytoplasm of the host cell to plastids has never been observed, despite the acknowledged role of RNA localization in the regulation of genetic expression.

Very recently, studies showing RNA targeting to chloroplasts has been disclosed in international patent application WO 2004/040973. However, the RNA sequences serving to translocate genes into chloroplasts are transformed with a CLS sequence (Chloroplast Localization Sequence) of viral origin, the sequences of which are preferentially chosen from ASBVd, PLMVd, CChMVd, CChMVd or else ELVd virus sequences, and are never nontransformed and/or endogenous RNA sequences as in the present invention.

The inventors have now demonstrated that transcripts of certain nuclear genes are localized in plastids, in cells of various plant species. The inventors have thus demonstrated, for the first time, that RNAs transcribed in the nucleus of plant cells can be translocated to plastids, in particular to chloroplasts.

The inventors have also demonstrated that an RNA of interest can be targeted specifically to a plant cell plastid by fusing this RNA of interest with a transcript of a nuclear gene detected in plastids. The transformation of plant cells with such a construct therefore makes it possible to translocate the RNA of interest to a plastid, and then to express, in the plastid, the protein encoded by this RNA of interest.

The mechanisms for targeting mRNA to plastids that has been identified by the inventors therefore represents an alternative to transformation of the plastid genome, in particular of the chloroplast genome, used up until now for a directed production of recombinant proteins in these organelles.

### DEFINITIONS

In the context of the present application, the term “nucleic acid” is intended to mean a phosphate ester of a polymeric form of ribonucleosides (adenosine, guanosine, uridine or cytidine; “RNA molecules”) or of deoxyribonucleosides (deoxyadenosine, deoxyguanosine, deoxythymidine or deoxycytidine; “DNA molecules”), or any phosphoester analog thereof, such as phosphorothioates and thioesters, in single-stranded form or in double-stranded form.

A “targeting nucleic acid” is a DNA or RNA molecule, the transcribed sequence of which is that of a gene which is localized in the nucleus of a plant cell (i.e. a nuclear gene), and which produces by transcription a messenger RNA (mRNA) which is translocated from the nucleus to a plastid of said plant cell.

The expression “transcribed sequence” denotes an RNA sequence which can be obtained by transcription of a DNA sequence, or which has the sequence of a reference RNA sequence. Thus, if the targeting nucleic acid is a DNA molecule, the transcribed sequence is an mRNA sequence which derives from the sequence of the DNA molecule. If the targeting nucleic acid is already an RNA molecule, the transcribed sequence is then that of the RNA molecule.

A targeting nucleic acid is therefore characterized by a transcribed sequence which is that of an mRNA of a nuclear gene, said mRNA being naturally detectable in a plastid of a plant cell. Said mRNA can have a predominantly cytoplasmic localization or a predominantly plastidial localization, or can be localized in equivalent amounts in the cytoplasm and in a plastid of the cell. Preferably, said mRNA has a predominantly plastidial localization; its concentration in a plastid is therefore greater than its cytoplasmic concentration. Said targeting nucleic acid is therefore characterized by a transcribed sequence which is that of an mRNA of a nuclear gene, said mRNA being characterized by a concentration in a plastid which is greater than its cytoplasmic concentration.

A “nucleic acid of interest” denotes a DNA or RNA molecule, the transcribed sequence (if it is a DNA molecule) of which it is desired to target to a plastid of a plant cell, or which (if it is an RNA molecule) it is desired to target to a plastid of a plant cell.

The term “plastid” denotes an ovoid or spherical organelle of a few microns in length and delimited by a double membrane or envelope. Plastids are specific to plant cells and to some protists. The role of these organelles is the synthesis or storage of molecules. Plastids include the chloroplast, which is the organelle in which photosynthesis takes place, the amyloplast, where the storage of starch occurs, the etioplast present in non-chlorophyll-containing tissues such as roots, the gerontoplast present in senescent tissues, the chromoplast which accumulates pigments, and the proplastid which is the origin of the other plastids.

### Method for Targeting to a Plastid

The inventors have demonstrated that, in plant cells, the mRNAs of certain nuclear genes have a subcellular localization in plastids. These results show that there exists a mechanism for translocation of mRNA from the nucleus and/or the cytosol of plant cells to plastids. In addition, constructs comprising such an mRNA fused with a nucleic acid sequence of interest are also found in plastids. These results therefore show that the transcripts of these nuclear genes constitute sequences for targeting to plastids.

The use of nuclear genes of which a transcript is localized in plastids is particularly useful for translocating nucleic acid sequences of interest, in particular RNA sequences, to specific subcellular plastid compartments.

The invention therefore relates to a method for targeting an RNA of interest to a plastid of a plant cell, said method comprising the transformation of a plant cell with a nucleic acid of interest linked to a targeting nucleic acid, the transcribed sequence of which is that of an mRNA of a nuclear gene, said mRNA being detectable in a plastid.

Preferably, the transcribed sequence of the targeting nucleic acid is the sequence of an mRNA of a nuclear gene which is endogenous to said plant cell.

The plastid can be selected from the group consisting of a chloroplast, an amyloplast, an etioplast, a gerontoplast, a chromoplast and a proplastid. Preferably, said plastid is a chloroplast.

The detection of a given mRNA in a plastid of a plant cell is within the scope of those skilled in the art. It is, for example, possible to extract the RNAs from plastids isolated from plant cells, and to search for the presence of a given mRNA by hybridization with a probe specific for the mRNA. The amount of mRNA isolated in a plastid can be compared with the amount present in the rest of the cell, i.e. the cytosol and all the organelles other than the plastid(s) considered, or with the amount present in the total RNA extracted from whole cells (cytoplasmic concentration). Preferably, said mRNA is characterized by a concentration in a plastid which is greater than its cytoplasmic concentration. More preferably, the concentration of said mRNA in a plastid is at least twice its cytoplasmic concentration. The respective concentrations of the mRNA in the plastid and in the cytoplasm can be determined in accordance with the methods described in the present application.

The inventors have thus identified, by means of a screening study on chips, a reproducible list of nuclear messengers highly enriched in plastid fractions, in particular chloroplast fractions, of plant cells. These mRNAs, and also the genomic DNA sequences or the cDNAs of these nuclear genes—once transcribed—therefore constitute nucleic acids for targeting an RNA to which they are linked, to a plastid compartment.

Preferably, a targeting nucleic acid according to the invention has a DNA or RNA sequence, the transcribed sequence of which is that of an mRNA of a nuclear gene selected from the group consisting of one of the genes identified in one of tables I, II, III or IV. The TAIR accession number is the number for accession to the gene identified in the TAIR database (The Arabidopsis Information Resource; http://www.arabidopsis.org) which was formed from the sequencing of the Arabidopsis thaliana genome.

TABLE I

| TAIR | |
|---|---|
| accession | |
| No. | Description (homologous genes identified in other organisms) |
| At3g03480 | transferase family similar to hypersensitivity-related gene GB: CAA64636 |
| | [Nicotiana tabacum]; contains Pfam transferase family domain PF00248 |
| At5g47250 | disease resistance protein (CC-NBS-LRR class), putative domain signature |
| | CC-NBS-LRR exists, suggestive of a disease resistance protein. |
| At5g08100 | Asparaginase |
| At2g33840 | tyrosyl-tRNA synthetase-related |
| At5g16770 | myb DNA-binding protein(AtMYB9) |
| At3g52520 | hypothetical protein |
| At3g50440 | hydrolase, alpha/beta fold family similar to ethylene-induced esterase [Citrus |
| | sinensis] GI: 14279437, polyneuridine aldehyde esterase [Rauvolfia serpentina] |
| | GI: 6651393; contains Pfam profile PF00561: hydrolase, alpha/beta fold family |
| At4g16920 | disease resistance protein (TIR-NBS-LRR class), putative domain signature |
| | TIR-NBS-LRR exists, suggestive of a disease resistance protein. |
| At5g65850 | F-box protein family |
| At5g54920 | expressed protein strong similarity to unknown protein (pir |
| At4g29905 | expressed protein |
| At3g06920 | pentatricopeptide (PPR) repeat-containing protein low similarity to fertility |
| | restorer [Petunia x hybrida] GI: 22128587; contains Pfam profile PF01535: |
| | PPR repeat |
| At5g39730 | avirulence induced gene (AIG) - like protein AIG2 PROTEIN, Arabidopsis |
| | thaliana, SWISSPROT: AIG2_ARATH |
| At4g23030 | MATE efflux protein - related contains Pfam profile PF01554: Uncharacterized |
| | membrane protein family |
| At4g10510 | subtilisin-like serine protease contains similarity to subtilase; SP1 GI: 9957714 |
| | from [Oryza sativa] |
| At3g13290 | transducin/WD-40 repeat protein family contains 2 WD-40 repeats (PF00400); |
| | autoantigen locus HUMAUTANT (GI: 533202) [Homo sapiens] and autoantigen |
| | locus HSU17474 (GI: 596134) [Homo sapiens] |
| At3g43380 | hypothetical protein hypothetical proteins - Arabidopsis thaliana |
| At5g08520 | expressed protein contains similarity to I-box binding factor |
| At4g07340 | contains similarity to Xenopus laevis replication protein A1 (SW: RFA1_XENLA) |
| At5g47790 | expressed protein |
| At5g05670 | signal recognition particle receptor beta subunit-related protein |
| At1g78890 | expressed protein |
| At1g17230 | leucine rich repeat protein family contains protein kinase domain, |
| | Pfam: PF00069; contains leucine-rich repeats, Pfam: PF00560 |
| At5g56160 | sec14 cytosolic factor family (phosphoglyceride transfer protein family) similar |
| | to SEC14 cytosolic factor (SP: P45816) [Candida lipolytica] |
| At1g07620 | GTP-binding protein - related similar to GB: M24537 from [Bacillus subtilis] |
| At5g45830 | tumor-related protein-like |
| At4g00730 | homeodomain protein AHDP |
| At3g59200 | F-box protein family contains F-box domain Pfam: PF00646 |
| At1g26930 | Kelch repeat containing F-box protein family contains Pfam: PF01344 Kelch |
| | motif, Pfam: PF00646 F-box domain |
| At1g79350 | expressed protein |
| At1g53290 | galactosyltransferase family contains Pfam profile: PF01762 |
| | galactosyltransferase; contains similarity to Avr9 elicitor response protein |
| | GI: 4138265 from [Nicotiana tabacum] |
| At3g07440 | expressed protein est hits to genscan model |
| At4g07750 | transposon protein-related similar to Arabidopsis thaliana putative En/Spm |
| | transposon protein (GB: AC005396) |
| At1g02410 | expressed protein contains similarity to cytochrome c oxidase assembly protein |
| | cox11 GI: 1244782 from [Saccharomyces cerevisiae] |
| At2g19750 | 40S ribosomal protein S30 (RPS30A) |
| At5g24600 | expressed protein similar to unknown protein (pir |
| At2g01710 | DnaJ protein family simlar to AHM1 [Triticum aestivum] GI: 6691467; contains |
| | Pfam profile PF00226: DnaJ domain |
| At5g38120 | 4-coumarate: CoA ligase (4-coumaroyl-CoA synthase) family similar to 4CL2, |
| | Arabidopsis thaliana [gi: 12229665], 4CL1, Nicotiana tabacum [gi: 12229631]; |
| | contains Pfam AMP-binding enzyme domain PF00501 |
| At2g44630 | Kelch repeat containing F-box protein family similar to SKP1 interacting partner |
| | 6 [Arabidopsis thaliana] GI: 10716957; contains Pfam profiles PF00646: F-box |
| | domain, PF01344: Kelch motif |
| At5g13350 | auxin-responsive - like protein Nt-gh3 deduced protein, Nicotiana tabacum, |
| | EMBL: AF123503 |
| At5g47200 | GTP-binding protein, putative similar to GTP-binding protein GI: 303750 from |
| | [Pisum sativum] |
| At2g28290 | SNF2 domain/helicase domain-containing protein similar to transcriptional |
| | activator HBRM [Homo sapiens] GI: 414117; contains Pfam profiles PF00271: |
| | Helicase conserved C-terminal domain, PF00176: SNF2 family N-terminal |
| | domain |
| At5g52610 | F-box protein family contains F-box domain Pfam: PF00646 |
| At4g30230 | hypothetical protein |
| At1g71030 | myb family transcription factor similar to MybHv5 GI: 19055 from [Hordeum |
| | vulgare] |
| At5g66230 | expressed protein similar to unknown protein (emb |
| At5g35180 | expressed protein |
| At2g10850 | envelope-related protein identical to GB: AAD20656 |
| At5g56670 | 40S ribosomal protein S30 (RPS30C) |
| At1g10660 | expressed protein |
| At3g01810 | expressed protein similar to unknown protein |
| At5g49160 | DNA (cytosine-5)-methyltransferase (DNA methyltransferase) (DNA metase) |
| | (sp |
| At5g66140 | 20S proteasome alpha subunit D2 (PAD2) (gb |
| At5g50250 | 31 kDa ribonucleoprotein, chloroplast (RNA-binding protein RNP-T/RNA- |
| | binding protein 1/2/3/RNA-binding protein cp31), putative similar to SP |
| At2g40510 | 40S ribosomal protein S26 (RPS26A) |
| At1g15700 | ATP synthase gamma-subunit-related similar to ATP synthase gamma-subunit |
| | GI: 21241 from [Spinacia oleracea] |
| At1g80300 | adenine nucleotide translocase identical to adenine nucleotide translocase |
| | GB: Z49227 [Arabidopsis thaliana] (FEBS Lett. 374 (3), 351-355 (1995)) |
| At1g17260 | ATPase 10, plasma membrane-type (proton pump 10) (proton-exporting |
| | ATPase), putative strong similarity to SP |
| At4g20160 | Glu-rich protein mature-parasite-infected erythrocyte surface antigen MESA, |
| | Plasmodium falciparum, PIR2: A45605 |
| At4g15440 | hydroperoxide lyase (HPOL) like protein |
| At4g22260 | alternative oxidase, putative (IMMUTANS) identical to IMMUTANS from |
| | Arabidopsis thaliana [gi: 4138855]; contains Pfam profile PF01786 alternative |
| | oxidase |
| At3g27690 | light harvesting chlorophyll A/B binding protein, putative similar to chlorophyll |
| | A-B binding protein 151 precursor (LHCP) GB: P27518 from [Gossypium |
| | hirsutum] |
| At4g09040 | RNA recognition motif (RRM) - containing protein low similarity to enhancer |
| | binding protein-1; EBP1 [Entamoeba histolytica] GI: 8163877, SP |
| At1g08550 | violaxanthin de-epoxidase precursor, putative similar to EST gb |
| At3g56690 | calmodulin-binding protein identical to calmodulin-binding protein GI: 6760428 |
| | from [Arabidopsis thaliana] |
| At3g15640 | cytochrome c oxidase subunit Vb-related similar to cytochrome oxidase IV |
| | GB: 223590 [Bos taurus]; contains Pfam profile: PF01215 cytochrome c |
| | oxidase subunit Vb |
| At4g16155 | dihydrolipoamide dehydrogenase 2, plastidic (lipoamide dehydrogenase 2) |
| | (ptlpd2) identical to plastidic lipoamide dehydrogenase from Arabidopsis |
| | thaliana [gi: 7159284] |
| At3g48425 | endonuclease/exonuclease/phosphatase family similar to SP |
| At2g31670 | expressed protein |
| At4g26670 | expressed protein |
| At2g40060 | expressed protein |
| At1g03780 | expressed protein |
| At1g19100 | hypothetical protein low similarity to microrchidia [Homo sapiens] GI: 5410257 |
| At4g31530 | expressed protein hypothetical protein - Arabidopsis thaliana, PIR2: T04873 |
| At5g57460 | expressed protein |
| At3g59840 | expressed protein |
| At1g06380 | expressed protein similar to hypothetical protein GI: 6598642 from [Arabidopsis |
| | thaliana] |
| At4g11960 | hypothetical protein hypothetical protein F7H19.70 - Arabidopsis thaliana, |
| | PID: e1310057 |
| At1g78620 | expressed protein |
| At3g48730 | glutamate-1-semialdehyde 2,1-aminomutase 2 (GSA 2) (glutamate-1- |
| | semialdehyde aminotransferase 2) (GSA-AT 2) identical to GSA2 [SP |
| At5g63570 | glutamate-1-semialdehyde 2,1-aminomutase 1 (GSA 1) (glutamate-1- |
| | semialdehyde aminotransferase 1) (GSA-AT 1) identical to GSA 1 [SP |
| At5g23710 | hypothetical protein |
| At3g44780 | hypothetical protein |
| At3g22400 | lipoxygenase (LOX), putative similar to lipoxygenase gi: 8649004 [Prunus |
| | dulcis], gi: 1495802 and gi: 1495804 from [Solanum tuberosum] |
| At3g54400 | nucleoid DNA-binding - like protein nucleoid DNA-binding protein cnd41, |
| | chloroplast, common tobacco, PIR: T01996 |
| At5g07020 | proline-rich protein family |
| At4g28660 | photosystem II protein W - like photosystem II protein W, Porphyra purpurea, |
| | PIR2: S73268 |
| At1g32900 | starch synthase, putative similar to starch synthase SP: Q42857 from [Ipomoea |
| | batatas] |
| At2g15570 | thioredoxin M-type 3, chloroplast precursor (TRX-M3) identical to SP |
| At5g44020 | vegetative storage protein-related |
| | trnY&trnE |
| At1g59453 | hypothetical protein contains similarity to transcription factors |
| At1g12170 | F-box protein family contains F-box domain Pfam: PF00646 |
| At1g46768 | AP2 domain protein RAP2.1 identical to AP2 domain containing protein |
| | RAP2.1 GI: 2281627 from [Arabidopsis thaliana] |
| At1g13620 | hypothetical protein |
| At1g77720 | protein kinase family contains protein kinase domain, Pfam: PF00069 |
| At1g50970 | hypothetical protein |
| At1g35530 | DEAD/DEAH box helicase, putative low similarity to RNA helicase/RNAseIII |
| | CAF protein [Arabidopsis thaliana] GI: 6102610; contains Pfam profiles |
| | PF00270: DEAD/DEAH box helicase, PF00271: Helicase conserved C- |
| | terminal domain |
| At1g55570 | pectinesterase (pectin methylesterase) family similar to pectinesterase |
| | [Lycopersicon esculentum][GI: 1944575]; nearly identical to pollen-specific |
| | BP10 protein [SP |
| At1g14000 | protein kinase-related |
| At1g35500 | hypothetical protein |
| At1g21170 | hypothetical protein |
| At1g72330 | alanine aminotransferase, putative similar to alanine aminotransferase 2 SP |
| At1g18040 | cell division protein kinase, putative similar to cell division protein kinase 7 |
| | [Homo sapiens] SWISS-PROT: P50613 |
| At1g44318 | porphobilinogen synthase (delta-aminolevulinic acid dehydratase), putative |
| | similar to delta-aminolevulinic acid dehydratase (Alad) GI: 493019 [SP |
| At1g60250 | CONSTANS B-box zinc finger family protein contains similarity to zinc finger |
| | protein GI: 3618320 from [Oryza sativa] |
| At1g08340 | rac GTPase activating protein-related similar to rac GTPase activating protein |
| | 1 GI: 3695059 from [Lotus japonicus] |
| At1g27260 | hypothetical protein |
| At4g16840 | expressed protein |
| At4g38620 | transcription factor (MYB4)-related |
| At2g47460 | myb family transcription factor similar to myb-related DNA-binding protein |
| | GI: 1020155 from [Arabidopsis thaliana] |
| At2g18010 | auxin-induced (indole-3-acetic acid induced) protein family similar to auxin- |
| | induced protein TGSAUR22 (GI: 10185820) [Tulipa gesnerian]; similar to |
| | indole-3-acetic acid induced protein ARG7 (SP: P32295) [Phaseolus aureus] |
| At2g36840 | ACT domain-containing protein contains Pfam profile ACT domain PF01842 |
| At2g37080 | myosin heavy chain-related |
| At2g31280 | expressed protein |
| At3g57380 | expressed protein hypothetical protein T32G6.16 - Arabidopsis thaliana, |
| | PIR: T00820 |
| At3g12170 | DnaJ protein family similar to SP |
| At3g57250 | hypothetical protein |
| At3g51470 | protein phosphatase 2C (PP2C), putative protein phosphatase-2C, |
| | Mesembryanthemum crystallinum, EMBL: AF075580 |
| At3g45990 | actin depolymerising like protein Actin depolymerising factor 2, Arabidopsis |
| | thaliana, EMBL: ATU48939 |
| At3g42950 | Polygalacturonase, putative polygalacturonase, muskmelon, PIR: T08213 |
| At3g47970 | hypothetical protein |
| At4g23780 | hypothetical protein Arabidopsis hypothetical proteins |
| At3g20350 | expressed protein |
| At4g27620 | expressed protein |
| At4g29700 | nucleotide pyrophosphatase-related protein nucleotide pyrophosphatase, |
| | Oryza sativa, gb: T03293 |
| At4g33560 | expressed protein |
| At4g26440 | WRKY family transcription factor identical to WRKY transcription factor 34 |
| | (WRKY34) GI: 15990591 from [Arabidopsis thaliana] |
| At4g36900 | AP2 domain protein RAP2.10 Identical to GP: 2632063 and GP: 7270639 |
| | [Arabidopsis thaliana] |
| At4g02150 | importin alpha-2 subunit identical to importin alpha-2 subunit (Karyopherin |
| | alpha-2 subunit) (KAP alpha) SP: O04294 from [Arabidopsis thaliana] |
| At5g03310 | auxin-induced (indole-3-acetic acid induced) protein family similar to indole-3- |
| | acetic acid induced protein ARG7 (SP: P32295) [Vigna radiata] |
| At5g16730 | expressed protein predicted proteins - Arabidopsis thaliana and Oryza sativa |
| At5g22700 | F-box protein family contains F-box domain Pfam: PF00646 |
| At5g13400 | peptide transporter - like protein peptide transporter, Hordeum vulgare, |
| | EMBL: AF023472 |
| At5g22620 | expressed protein similar to unknown protein (dbj |
| At5g43800 | pseudogene, similar to gag-pol polyprotein (Ty1_Copia-element) [Glycine max] |
| | (GB: AAC64917) similar to gag/pol polyprotein [Arabidopsis thaliana] gi |
| At5g37450 | leucine-rich repeat transmembrane protein kinase, putative |
| At5g52410 | expressed protein |
| At5g14860 | glycosyltransferase family contains Pfam profile: PF00201 UDP-glucoronosyl |
| | and UDP-glucosyl transferase |
| At5g47520 | GTP-binding protein, putative similar to GTP-binding protein RAB11J |
| | GI: 1370160 from [Lotus japonicus] |
| At5g51360 | hypothetical protein |
| At5g22850 | protease-related protein |
| At2g37640 | expansin, putative (EXP3) identical to Alpha-expansin 3 precursor (At- |
| | EXP3)[Arabidopsis thaliana] SWISS-PROT: O80932; alpha-expansin gene |
| | family, PMID: 11641069 |
| At2g18040 | peptidyl-prolyl cis-trans isomerase-related similar to ESS1 (S. cerevisiae) and |
| | dodo (D. melanogaster.) |
| At3g49250 | expressed protein |
| At2g13150 | bZIP family transcription factor contains a bZIP transcription factor basic |
| | domain signature (PDOC00036) |
| At4g39690 | expressed protein |
| At4g34180 | expressed protein hypothetical protein slr2121, Synechocystis sp., |
| | PIR2: S75497 |
| At4g01350 | CHP-rich zinc finger protein, putative similar to A. thaliana CHP-rich zinc finger |
| | proteins see T10M13, GenBank accession number AF001308 functional |
| | catalog ID = 98 |
| At2g04450 | MutT/nudix family protein similar to SP |
| At1g24540 | cytochrome P450, putative similar to GB: AAB87111, similar to ESTs dbj |
| At5g38480 | 14-3-3 protein GF14 psi (grf3/RCI1) identical to 14-3-3 protein GF14 psi |
| | GI: 1168200, SP: P42644 |
| At2g07770 | hypothetical protein low similarity to KED [Nicotiana tabacum] GI: 8096269; |
| | contains Pfam profile PF03384: Drosophila protein of unknown function, |
| | DUF287 |
| At2g11930 | pseudogene, hypothetical protein and genefinder |
| At1g53730 | leucine-rich repeat transmembrane protein kinase 1, putative similar to |
| | GI: 3360289 from [Zea mays] (Plant Mol. Biol. 37 (5), 749-761 (1998)) |
| At1g61580 | 60S ribosomal protein L3 (RPL3B) identical to ribosomal protein GI: 806279 |
| | from [Arabidopsis thaliana] |
| At1g30450 | cation-chloride cotransporter, putative similar to cation-chloride co-transporter |
| | GB: AAC49874 GI: 2582381 from [Nicotiana tabacum], Cation-Chloride |
| | Cotransporter (CCC) Family Member, PMID: 11500563 |
| At1g76110 | expressed protein |
| At1g51300 | hypothetical protein |
| At1g17880 | transcription factor-related similar to transcription factor BTF3 homolog |
| | GI: 2982299 from [Picea mariana] |
| At1g04880 | expressed protein |
| At1g30840 | purine permease-related low similarity to purine permease [Arabidopsis |
| | thaliana] GI: 7620007; contains Pfam profiles PF03151: Domain of unknown |
| | function, DUF250, PF00892: Integral membrane protein |
| At1g54490 | exonuclease-related similar to 5′-3′ exonuclease GI: 1894792 from [Mus |
| | musculus] |
| At2g31320 | poly (ADP-ribose) polymerase-related |
| At2g38500 | expressed protein |
| At2g02180 | TOM3 protein annotation temporarily based on supporting cDNA gi |
| At2g45070 | transport protein SEC61 beta-subunit-related |
| At2g16860 | expressed protein |
| At4g31980 | expressed protein EREBP-4 homolog, Arabidopsis thaliana |
| At3g15700 | hypothetical protein similar to N-term of NBS/LRR disease resistance protein |
| | GB: AAC26125 [Arabidopsis thaliana]; contains Pfam profile: PF00931 NB- |
| | ARC domain |
| At3g05130 | hypothetical protein |
| At3g01840 | protein kinase family contains protein kinase domain, Pfam: PF00069 |
| At3g21933 | pseudogene contains Pfam profile: PF01657 Domain of unknown function |
| At3g17470 | calcium-binding EF-hand family protein contains INTERPRO: IPR002048 |
| | calcium-binding EF-hand domain |
| At3g60520 | expressed protein |
| At3g42430 | hypothetical protein various predicted proteins, Arabidopsis thaliana |
| At3g02480 | expressed protein similar to ABA-inducible protein [Fagus sylvatica] |
| | GI: 3901016, cold-induced protein kin1 [Brassica napus] GI: 167146 |
| At3g08560 | vacuolar ATP synthase subunit E-related similar to vacuolar ATP synthase |
| | subunit E GB: Q39258 [Arabidopsis thaliana] |
| At3g62260 | protein phosphatase 2C (PP2C), putative phosphoprotein phosphatase (EC |
| | 3.1.3.16) 1A-alpha - Homo sapiens, PIR: S22423 |
| At3g53330 | plastocyanin-like domain containing protein similar to mavicyanin SP: P80728 |
| | from [Cucurbita pepo] |
| At4g15040 | subtilisin-like serine protease contains similarity to prepro-cucumisin GI: 807698 |
| | from [Cucumis melo] |
| At4g10740 | hypothetical protein |
| At4g22560 | expressed protein predicted proteins, Arabidopsis thaliana |
| At4g29750 | expressed protein predicted proteins, Arabidopsis thaliana |
| At4g37130 | proline-rich protein-related |
| At4g19210 | RNase L inhibitor protein, putative similar to 68 kDa protein HP68 GI: 16755057 |
| | from [Triticum aestivum] |
| At5g41040 | transferase family similar to hypersensitivity-related gene product HSR201 - |
| | Nicotiana tabacum, EMBL: X95343; contains Pfam transferase family domain |
| | PF00248 |
| At5g37690 | lipase family similar to family II lipase EXL3 (GI: 15054386), EXL1 |
| | (GI: 15054382), EXL2 (GI: 15054384) [Arabidopsis thaliana] |
| At5g46000 | jacalin lectin family similar to myrosinase-binding protein homolog [Arabidopsis |
| | thaliana] GI: 2997767; contains Pfam profile PF01419 jacalin-like lectin |
| | domain |
| At2g22500 | mitochondrial carrier protein family contains Pfam profile: PF00153 |
| | mitochondrial carrier protein |
| At5g54310 | ARF GAP-like zinc finger-containing protein (ZIGA3) almost identical to ARF |
| | GAP-like zinc finger-containing protein ZIGA3 GI: 10441352 from [Arabidopsis |
| | thaliana] |
| At5g15490 | UDP-glucose dehydrogenase-related protein UDP-glucose 6-dehydrogenase - |
| | Glycine max, EMBL: U53418 |
| At4g13510 | ammonium transport protein (AMT1) |
| At5g55350 | long-chain-alcohol O-fatty-acyltransferase (wax synthase) family contains |
| | similarity to wax synthase wax synthase - Simmondsia chinensis, |
| | PID: g5020219 similar to wax synthase [gi: 5020219] from Simmondsia |
| | chinensis |
| At4g02630 | protein kinase family contains protein kinase domain, Pfam: PF00069; contains |
| | serine/threonine protein kinase domain, INTERPRO: IPR002290 |
| At1g56100 | hypothetical protein |
| At1g32430 | F-box protein family contains F-box domain Pfam: PF00646 |
| At1g74150 | Kelch repeat-containing protein low similarity to rngB protein, Dictyostelium |
| | discoideum, PIR: S68824; contains Pfam profile PF01344: Kelch motif |
| At1g69770 | chromomethylase-related similar to chromomethylase GB: AAB95486 |
| | [Arabidopsis arenosa] |
| At2g39590 | 40S ribosomal protein S15A (RPS15aC) |
| At3g30810 | hypothetical protein |
| At5g18620 | DNA-dependent ATPase, putative similar to DNA-dependent ATPase SNF2H |
| | [Mus musculus] GI: 14028669; contains Pfam profiles PF00271: Helicase |
| | conserved C-terminal domain, PF00176: SNF2 family N-terminal domain, |
| | PF00249: Myb-like DNA-binding domain |
| At1g55930 | CBS/transporter associated domain-containing protein contains Pfam profiles |
| | PF00571: CBS domain, PF03471: Transporter associated domain, PF01595: |
| | Domain of unknown function |
| At1g62050 | expressed protein |
| At3g25940 | expressed protein |
| At1g26540 | expressed protein |
| At1g80050 | adenine phosphoribosyltransferase almost identical to adenine |
| | phosphoribosyltransferase GI: 1402894 from [Arabidopsis thaliana] |
| At1g59312 | hypothetical protein |
| At1g64960 | expressed protein |
| At1g03370 | C2 domain/GRAM domain-containing protein low similarity to SP |
| At1g56290 | expressed protein |
| At1g03590 | protein phosphatase 2C (PP2C) similar to GB: AAB97706 |
| At4g17910 | hypothetical protein predicted protein, Saccharomyces cerevisiae, |
| | PIR2: S56868 |
| At5g35340 | hypothetical protein |
| At2g33580 | protein kinase-related contains a protein kinase domain profile (PDOC00100) |
| At2g44190 | expressed protein |
| At2g18480 | mannitol transporter, putative similar to mannitol transporter [Apium graveolens |
| | var. dulce] GI: 12004316; contains Pfam profile PF00083: major facilitator |
| | superfamily protein |
| At2g46310 | AP2 domain transcription factor, putative |
| At3g27590 | hypothetical protein |
| At3g09600 | myb family transcription factor contains Pfam profile: PF00249 myb-like DNA- |
| | binding domain |
| At3g12870 | hypothetical protein similar to oxidoreductases |
| At3g26090 | expressed protein |
| At3g13224 | RNA recognition motif (RRM) - containing protein contains InterPro entry |
| | IPR000504: RNA-binding region RNP-1 (RNA recognition motif) (RRM) |
| At3g20475 | DNA mismatch repair MutS family similar to SP |
| At3g54220 | scarecrow transcription factor (SCR) |
| At3g61510 | 1-aminocyclopropane-1-carboxylate synthase (ACC synthase), putative similar |
| | to ACC synthases from Citrus sinensis [GI: 6434142], Cucumis melo |
| | [GI: 695402], Cucumis sativus [GI: 3641645] |
| At3g46020 | RNA-binding protein, putative similar to Cold-inducible RNA-binding protein |
| | (Glycine-rich RNA-binding protein CIRP) from {Homo sapiens} SP |
| At3g20280 | expressed protein contains Pfam profile: PF00628 PHD-finger, implications for |
| | chromatin-mediated transcriptional regulation |
| At4g28780 | GDSL-motif lipase/hydrolase protein similar to family II lipase EXL3 |
| | (GI: 15054386), EXL1 (GI: 15054382), EXL2 (GI: 15054384) [Arabidopsis |
| | thaliana]; contains Pfam profile PF00657: Lipase/Acylhydrolase with GDSL- |
| | like motif |
| At4g13650 | pentatricopeptide (PPR) repeat-containing protein contains Pfam profile |
| | PF01535: PPR repeat |
| At4g09580 | expressed protein hypothetical protein - Arabidopsis thaliana, PIR2: B71448 |
| At4g18040 | translation initiation factor eIF4E |
| At4g13810 | disease resistance protein family (LRR) contains leucine rich-repeat domains |
| | Pfam: PF00560, INTERPRO: IPR001611; similar to disease resistance protein |
| | [Lycopersicon esculentum] gi |
| At5g04220 | C2 domain-containing protein GC donor splice site at exon 3; similar to Ca2+- |
| | dependent lipid-binding protein (CLB1) GI: 2789434 from [Lycopersicon |
| | esculentum] |
| At5g07650 | formin homology 2 (FH2) domain-containing protein contains formin homology |
| | 2 domain, Pfam: PF02128 |
| At5g58430 | leucine zipper-containing protein leucine zipper-containing protein, |
| | Lycopersicon esculentum, PIR: S21495 |
| At5g18240 | transfactor-related protein |
| At5g48690 | hypothetical protein |
| At5g66460 | glycosyl hydrolase family 5/cellulase ((1-4)-beta-mannan endohydrolase) |
| At5g60060 | F-box protein family various predicted proteins, Arabidopsis thaliana; similar to |
| | SKP1 interacting partner 2 (SKIP2) TIGR_Ath1: At5g67250 |
| At5g14070 | glutaredoxin protein family contains INTERPRO Domain IPR002109, |
| | Glutaredoxin (thioltransferase) |
| At4g10020 | short-chain dehydrogenase/reductase family protein similar to sterol-binding |
| | dehydrogenase steroleosin GI: 15824408 from [Sesamum indicum] |
| At5g20730 | auxin response transcription factor (ARF7) identical to auxin response factor 7 |
| | GI: 4104929 from [Arabidopsis thaliana] |
| At5g65630 | bromodomain-containing protein similar to 5.9 kb fsh membrane protein |
| | [Drosophila melanogaster] GI: 157455; contains Pfam profile PF00439: |
| | Bromodomain |
| At2g02290 | hypothetical protein and genefinder |
| At1g78300 | 14-3-3 protein GF14 omega (grf2) identical to GF14omega isoform GI: 487791 |
| | from [Arabidopsis thaliana] |
| At1g61960 | expressed protein similar to hypothetical protein GI: 5541664 from [Arabidopsis |
| | thaliana] |
| At2g14630 | hypothetical protein contains Pfam profile PF03004: Plant transposase |
| | (Ptta/En/Spm family) |
| At5g16230 | acyl-[acyl-carrier-protein] desaturase (stearoyl-ACP desaturase), putative |
| | similar to Acyl-[acyl-carrier protein] desaturase from Spinacia oleracea SP |
| At1g22170 | expressed protein contains similarity to phosphoglycerate mutases |
| At4g08320 | tetratricopeptide repeat (TPR)-containing protein glutamine-rich |
| | tetratricopeptide repeat (TPR) containing protein (SGT) - Rattus |
| | norvegicus, PID: e1285298 (SP |
| At5g49500 | SRP54 (signal recognition particle 54 KDa) protein |
| At2g01420 | auxin transport protein, putative similar to auxin transport protein |
| | PIN7 [Arabidopsis thaliana] gi |
| At3g49400 | transducin/WD-40 repeat protein family contains 4 WD-40 repeats (PF00400); |
| | low similarity (47%) to Agamous-like MADS box protein AGL5 (SP: P29385) |
| | {Arabidopsis thaliana} |
| AtCg00630 | psaJ: photosystem I subunit IX |
| At1g22210 | trehalose-6-phosphate phosphatase, putative similar to trehalose-6-phosphate |
| | phosphatase (AtTPPB) GI: 2944180 from [Arabidopsis thaliana]; contains Pfam |
| | profile PF02358: Trehalose-phosphatase |
| At1g68935 | expressed protein |
| At1g24625 | zinc finger protein 7, ZFP7 |
| At1g08100 | high-affinity nitrate transporter ACH2 identical to GB: AAC35884 from |
| | [Arabidopsis thaliana] (Plant J. 17 (5), 563-568 (1999)) |
| At1g70460 | protein kinase-related similar to C-terminal region has similarity to C-terminal |
| | region of protein kinase (APK1A) GB: Q06548 [Arabidopsis thaliana]; Pfam |
| | HMM hit: Eukaryotic protein kinase domain |
| At1g71750 | hypoxanthine ribosyl transferase-related similar to hypoxanthine ribosyl |
| | transferase GB: AAC46403 GI: 2689037 from [Vibrio parahaemolyticus] |
| At3g52910 | expressed protein growth-regulating factor 1, Oryza sativa, EMBL: AF201895 |
| At4g38240 | alpha-1,3-mannosyl-glycoprotein beta-1,2-N-acetylglucosaminyltransferase, |
| | putative similar to N-acetylglucosaminyltransferase I from Arabidopsis |
| | thaliana [gi: 5139335]; contains AT-AC non-consensus splice sites at intron 13 |
| At5g59613 | expressed protein |
| At2g19000 | expressed protein |
| At2g32400 | glutamate receptor family (GLR3.7)(GLR5) identical to Glr5 [Arabidopsis |
| | thaliana] gi |
| At2g46050 | pentatricopeptide (PPR) repeat-containing protein contains |
| | INTERPRO: IPR002885 PPR repeats |
| At3g02810 | protein kinase family contains protein kinase domain, Pfam: PF00069 |
| At3g09080 | transducin/WD-40 repeat protein family contains 8 WD-40 repeats; similar to |
| | JNK-binding protein JNKBP1 (GP: 6069583) [Mus musculus] |
| At3g04660 | F-box protein family contains F-box domain Pfam: PF00646 |
| At3g06160 | transcriptional factor B3 family contains Pfam profile PF02362: B3 DNA binding |
| | domain |
| At3g61450 | syntaxin of plants 73 (SYP73) annotation temporarily based on supporting |
| | cDNA gi |
| At3g12540 | hypothetical protein |
| At3g26800 | hypothetical protein |
| At3g15510 | No apical meristem (NAM) protein family contains Pfam PF02365: No apical |
| | meristem (NAM) domain; similar to jasmonic acid 2 GB: AAF04915 from |
| | [Lycopersicon esculentum] |
| At3g56790 | hypothetical protein hypothetical protein F27K19.110 - Arabidopsis thaliana, |
| | PIR: T49205 |
| At4g30870 | hypothetical protein hypothetical protein, Schizosaccharomyces pombe, |
| | PID: E322903 |
| At4g00750 | dehydration-induced protein family similar to early-responsive to dehydration |
| | stress ERD3 protein [Arabidopsis thaliana] GI: 15320410; contains Pfam profile |
| | PF03141: Putative methyltransferase |
| At4g37400 | cytochrome P450 family similar to cytochrome P450 monooxygenase |
| | CYP91A2, Arabidopsis thaliana, D78607 |
| At4g15890 | expressed protein |
| At4g09510 | neutral invertase like protein Daucus carota mRNA, PID: e1372926 |
| At4g14720 | expressed protein |
| At5g58000 | expressed protein similar to unknown protein (gb |
| At5g39790 | expressed protein 5′-AMP-ACTIVATED PROTEIN KINASE, BETA-1 SUBUNIT, |
| | pig, SWISSPROT: AAKB_PIG |
| At5g01390 | DnaJ protein family similar to SP |
| At5g53210 | bHLH protein family contains similarity to helix-loop-helix DNA-binding protein |
| At5g51030 | short-chain dehydrogenase/reductase family protein contains INTERPRO |
| | family IPR002198 short chain dehydrogenase/reductase SDR family |
| At5g67540 | glycosyl hydrolase family 43 contains similarity to xylanase GI: 2645416 from |
| | [Caldicellulosiruptor saccharolyticus] |
| At5g50030 | expressed protein contains similarity to pollen-specific protein Bnm1 Brassica |
| | napus GI: 1857671; contains Pfam profile PF04043: Plant invertase/pectin |
| | methylesterase inhibitor |
| At5g05190 | expressed protein similar to unknown protein (emb |
| At3g12600 | MutT/nudix family protein contains Pfam profile PF00293: NUDIX domain |
| At3g54180 | cell division control protein 2 homolog B (CDC2B) identical to cell division |
| | control protein 2 homolog B [Arabidopsis thaliana] SWISS-PROT: P25859 |
| At5g01640 | expressed protein prenylated Rab acceptor 1 - Homo sapiens, |
| | EMBL: AJ133534 |
| At5g53230 | hypothetical protein similar to unknown protein (pir |
| At2g33530 | serine carboxypeptidase-related |
| At2g43690 | receptor lectin kinase, putative similar to receptor-like kinase LECRK1 |
| | [Arabidopsis thaliana] gi |
| At3g09110 | hypothetical protein |
| At4g27130 | translation initiation factor |
| At1g60220 | Ulp1 protease family contains Pfam profile PF02902: Ulp1 protease family, C- |
| | terminal catalytic domain |
| At1g49140 | NADH-ubiquinone oxidoreductase 12 kD subunit-related annotation |
| | temporarily based on supporting cDNA gi |
| At1g52700 | hypothetical protein contains similarity to lysophospholipase GI: 1552244 from |
| | [Rattus norvegicus] |
| At4g39430 | hypothetical protein |
| At1g50980 | F-box protein family contains F-box domain Pfam: PF00646 |
| At4g35600 | protein kinase family contains protein kinase domain, Pfam: PF00069 |
| At2g18980 | peroxidase, putative identical to peroxidase ATP22a [Arabidopsis thaliana] gi |
| At2g27410 | hypothetical protein |
| At2g14520 | CBS domain containing protein contains Pfam profiles PF00571: CBS domain, |
| | PF01595: Domain of unknown function |
| At2g33770 | ubiquitin-conjugating enzyme family low similarity to ubiquitin-conjugating BIR- |
| | domain enzyme APOLLON [Homo sapiens] GI: 8489831, ubiquitin-conjugating |
| | enzyme [Mus musculus] GI: 3319990; contains Pfam profile PF00179: |
| | Ubiquitin-conjugating enzyme |
| At2g24500 | C2H2-type zinc finger protein-related likely a nucleic acid binding protein |
| At2g19190 | light repressible receptor protein kinase, putative similar to light repressible |
| | receptor protein kinase [Arabidopsis thaliana] gi |
| At2g18070 | hypothetical protein |
| At2g41970 | protein kinase, putative similar to Pto kinase interactor 1 (serine/threonine |
| | protein kinase) [Lycopersicon esculentum] gi |
| At3g30875 | pseudogene, putative multidrug resistance protein similar to multidrug |
| | resistance protein 1 homolog GB: T06165 GI: 7442649 from [Hordeum vulgare] |
| At3g29618 | pseudogene, similar to mudrA of transposon = MuDR“ (MuDr-element) [Zea |
| | mays] (GB: AAA21566) similar to Mutator-like transposase GB: AAD25591 |
| | from [Arabidopsis thaliana]” |
| At3g28030 | UV hypersensitive protein (UVH3) annotation temporarily based on supporting |
| | cDNA gi |
| At3g59410 | protein kinase like GCN2 - Saccharomyces cerevisiae, EMBL: M27082 |
| At3g56490 | protein kinase C inhibitor-related protein protein kinase C inhibitor - Zea mays, |
| | PIR: S45368 |
| At3g29280 | hypothetical protein |
| At3g15310 | expressed protein |
| At3g26600 | expressed protein |
| At3g25100 | Cdc45-related protein similar to Cdc45 GB: AAC67520 [Xenopus laevis] (EMBO |
| | J. 17, 5699-5707 (1998)) (required for the initiation of eukaryotic DNA |
| | replication) |
| At3g26295 | pseudogene, cytochrome P450 |
| At3g22780 | DNA binding protein-related identical to putative DNA binding protein |
| | GB: AAF27433 from [Arabidopsis thaliana] |
| At3g05050 | cyclin-dependent protein kinase-related similar to cyclin-dependent kinase |
| | GB: CAA65979 from [Medicago sativa] |
| At3g47900 | expressed protein various predicted proteins |
| At3g29570 | hypothetical protein |
| At3g13310 | DnaJ protein family similar to J11 protein [Arabidopsis thaliana] GI: 9843641; |
| | contains Pfam profile: PF00226 DnaJ domain |
| At4g00770 | expressed protein |
| At4g38270 | glycosyltransferase family 8 contains Pfam profile: PF01501 glycosyl |
| | transferase family 8 |
| At4g11930 | hypothetical protein |
| At4g36560 | hypothetical protein |
| At4g08470 | mitogen-activated protein kinase, putative similar to mitogen-activated protein |
| | kinase [Arabidopsis thaliana] gi |
| At4g40000 | proliferating-cell nucleolar antigen - like protein proliferating-cell nucleolar |
| | antigen, Saccharomyces cerevisiae, PIR2: S45758 |
| At4g04180 | vesicle transfer ATPase-related |
| At5g53710 | expressed protein |
| At5g03890 | hypothetical protein predicted protein, Arabidopsis thaliana |
| At5g61300 | hypothetical protein predicted protein, Arabidopsis thaliana |
| At5g22510 | alkaline/neutral invertase |
| At5g48660 | hypothetical protein contains similarity to unknown protein (gb |
| At5g47280 | disease resistance protein (NBS-LRR class), putative domain signature NBS- |
| | LRR exists, suggestive of a disease resistance protein. |
| At2g47580 | small nuclear ribonucleoprotein (spliceosomal protein) U1A identical to |
| | GB: Z49991 U1snRNP-specific protein [Arabidopsis thaliana] |
| At1g05200 | glutamate receptor family (GLR3.4) plant glutamate receptor family, |
| | PMID: 11379626 |
| At2g18240 | integral membrane protein-related |
| At5g64470 | expressed protein similar to unknown protein (gb |
| At1g31300 | expressed protein similar to hypothetical protein GB: AAF24587 GI: 6692122 |
| | from [Arabidopsis thaliana] |
| At3g59530 | strictosidine synthase-related similar to strictosidine synthase [Rauvolfia |
| | serpentina][SP |
| At4g29600 | cytidine deaminase 7 |
| At3g21130 | F-box protein family contains Pfam profile: PF00646 F-box domain |
| At3g20000 | membrane import protein-related similar to membrane import protein |
| | GB: AAF20172 GI: 6636407 [Drosophila melanogaster] |
| At2g24520 | ATPase, plasma membrane-type (proton pump), putative strong similarity to P- |
| | type H(+)-transporting ATPase from [Phaseolus vulgaris] GI: 758250, |
| | [Lycopersicon esculentum] GI: 1621440, SP |
| At1g09410 | pentatricopeptide (PPR) repeat-containing protein contains |
| | INTERPRO: IPR002885 PPR repeats |
| At1g67460 | hypothetical protein |
| At3g06560 | poly(A) polymerase-related similar to polynucleotide adenylyltransferase |
| | GB: S17875 from [Bos taurus] (Nature (1991) 353 (6341), 229-234) |
| At2g42030 | C3HC4-type zinc finger protein family contains Pfam profile: PF00097 zinc |
| | finger, C3HC4 type (RING finger) |
| At1g22630 | auxin-regulated protein |
| At3g42600 | hypothetical protein |
| At2g29340 | short-chain dehydrogenase/reductase family protein similar to tropinone |
| | reductase-I GI: 424160 from [Datura stramonium] |
| At1g22600 | seed maturation protein PM27-related similar to seed maturation protein PM27 |
| | GI: 4836403 from [Glycine max] |
| At1g48380 | root hairless 1 (RHL1) similar to root hairless 1 GI: 3219355 from [Arabidopsis |
| | thaliana] |
| At1g72960 | root hair defective-related similar to root hair defective 3 GI: 1839188 from |
| | [Arabidopsis thaliana] |
| At1g24530 | transducin/WD-40 repeat protein family similar to Vegetatible incompatibility |
| | protein HET-E-1 (SP: Q00808) {Podospora anserina}; contains 7 WD-40 |
| | repeats (PF00400) |
| At1g48520 | Glu-tRNA(Gln) amidotransferase subunit B; nuclear gene for chloroplast |
| | product annotation temporarily based on supporting cDNA gi |
| At1g61370 | receptor protein kinase (IRK1)-related similar to receptor protein kinase (IRK1) |
| | GI: 836953 from [Ipomoea trifida] |
| At1g32310 | expressed protein |
| At2g47410 | transducin/WD-40 repeat protein family contains 5 WD-40 repeats (PF00400); |
| | similar to WDR protein, form B (GI: 14970593) [Mus musculus] |
| At1g14280 | phytochrome kinase substrate 1-related similar to phytochrome kinase |
| | substrate 1 GI: 5020168 from [Arabidopsis thaliana] |
| At1g75620 | hypothetical protein |
| At4g19420 | pectinacetylesterase family contains Pfam profile: PF03283 |
| | pectinacetylesterase |
| At5g27150 | sodium proton exchanger (NHX1) identical to Na+/H+ exchanger [Arabidopsis |
| | thaliana] gi |
| At2g06005 | expressed protein |
| At2g20000 | cell division cycle (CDC) protein-related low similarity to SP |
| At2g44170 | N-myristoyltransferase-related |
| At2g46100 | expressed protein |
| At3g63240 | endonuclease/exonuclease/phosphatase family similar to inositol |
| | polyphosphate 5-phosphatase I (GI: 10444261) and II (GI: 10444263) |
| | [Arabidopsis thaliana]; contains Pfam profile PF03372: |
| | Endonuclease/Exonuclease/phosphatase family |
| At3g25890 | AP2 domain transcription factor, putative |
| At3g62190 | DnaJ protein family similar to SP |
| At4g38210 | expansin, putative (EXP20) similar to alpha-expansin 3 GI: 6942322 from |
| | [Triphysaria versicolor]; alpha-expansin gene family, PMID: 11641069 |
| At4g04840 | expressed protein similar to transcriptional regulator |
| At4g35540 | hypothetical protein transcription factor IIIB chain BRF1, |
| | Saccharomyces cerevisiae, PIR2: A44072 |
| At4g28000 | hypothetical protein MSP1, Saccharomyces cerevisiae, PIR2: A49506 |
| At4g01760 | CHP-rich zinc finger protein, putative similar to T15B16.10 similar to A. thaliana |
| | CHP-rich proteins encoded by T10M13, GenBank accession number |
| | AF001308 |
| At5g52850 | pentatricopeptide (PPR) repeat-containing protein contains |
| | INTERPRO: IPR002885 PPR repeats |
| At5g15040 | hypothetical protein predicted proteins, Arabidopsis thaliana |
| At5g24030 | expressed protein contains similarity to unknown protein (pir |
| At5g50870 | ubiquitin-conjugating enzyme, putative strong similarity to ubiquitin conjugating |
| | enzyme [Lycopersicon esculentum] GI: 886679; contains Pfam profile |
| | PF00179: Ubiquitin-conjugating enzyme |
| At5g55430 | hypothetical protein |
| At5g06340 | diadenosine 5′,5′″-P1,P4-tetraphosphate hydrolase, putative similar to |
| | diadenosine 5′,5′″-P1,P4-tetraphosphate hydrolase from [Lupinus |
| | angustifolius] GI: 1888557, [Hordeum vulgare subsp. vulgare] GI: 2564253; |
| | contains Pfam profile PF00293: NUDIX domai? |
| At5g11050 | myb family transcription factor contains Pfam profile: PF00249 myb-like DNA |
| | binding domain |
| At1g75180 | expressed protein |
| At4g37010 | caltractin (centrin), putative similar to Caltractin (Centrin) SP: P41210 from |
| | [Atriplex nummularia] |
| At4g37020 | expressed protein |
| At5g43380 | serine/threonine protein phosphatase type on(TOPP7) |
| At4g20020 | putative DAG protein annotation temporarily based on supporting cDNA gi |
| At4g02670 | zinc finger protein-related similar to potato PCP1 zinc finger protein, GenBank |
| | accession number X82328 |
| At4g07720 | hypothetical protein |
| At2g29880 | hypothetical protein |
| At2g24740 | SET-domain transcriptional regulator family identical to SUVH8 [Arabidopsis |
| | thaliana] GI: 13517757; contains Pfam profiles PF00856: SET domain, |
| | PF05033: Pre-SET motif, PF02182: YDG/SRA domain |
| At2g02840 | hypothetical protein |
| AtCg00960 | rrn4.5S: 23S ribosomal RNA |
| At1g69420 | DHHC-type zinc finger domain-containing protein contains Pfam profile: |
| | PF01529: DHHC zinc finger domain |
| At1g31790 | pentatricopeptide (PPR) repeat-containing protein contains Pfam profile |
| | PF01535: PPR repeat |
| At1g48780 | hypothetical protein |
| At1g67150 | hypothetical protein |
| At1g55830 | expressed protein |
| At1g21480 | Exostosin family contains Pfam profile: PF03016 Exostosin family |
| At1g22050 | ubiquitin family contains INTERPRO: IPR000626 ubiquitin domain |
| At1g71080 | expressed protein |
| At1g71290 | F-box protein-related contains weak hit to TIGRFAM TIGR01640:F-box |
| | protein interaction domain contains weak hit to TIGRFAM TIGR01640:F-box |
| | protein interaction domain; |
| At2g11200 | F-box protein family |
| At2g38270 | expressed protein |
| At2g05400 | expressed protein |
| At2g04790 | expressed protein |
| At2g47540 | expressed protein and genefinder |
| At2g23470 | expressed protein |
| At3g30380 | hypothetical protein contains Pfam profile: PF00561 alpha/beta hydrolase fold |
| At3g08770 | lipid transfer protein 6 (ltp6) identical to GI: 8571927 |
| At3g58630 | expressed protein hypothetical protein F9F8.9 - Arabidopsis thaliana, |
| | EMBL: AC009991 |
| At3g17850 | protein kinase, putative similar to IRE (incomplete root hair elongation) |
| | [Arabidopsis thaliana] gi |
| At3g29190 | terpene synthase/cyclase family contains Pfam profile: PF01397 terpene |
| | synthase family |
| At4g26560 | calcineurin B-like protein, putative similar to calcineurin B-like protein 3 |
| | GI: 3309086 from [Arabidopsis thaliana] |
| At4g21840 | expressed protein CGI-131 protein, Homo sapiens, AF151889 |
| At4g36600 | late embryogenesis abundant (LEA) domain-containing protein low similarity to |
| | SP |
| At5g57220 | cytochrome P450, putative similar to Cytochrome P450 (SP: O65790) |
| | [Arabidopsis thaliana]; Cytochrome P450 (GI: 7415996) [Lotus japonicus] |
| At5g45700 | hypothetical protein |
| At5g17770 | NADH-cytochrome b5 reductase identical to NADH-cytochrome b5 reductase |
| | [Arabidopsis thaliana] GI: 4240116 |
| At5g49430 | transducin/WD-40 repeat protein family similar to WD-repeat protein 9 |
| | (SP: Q9NSI6) {Homo sapiens}; contains Pfam PF00400: WD domain, G-beta |
| | repeat (4 copies) |
| At5g59640 | serine/threonine-specific protein kinase - like putative protein serine/threonine |
| | kinase, Sorghum bicolor, EMBL: SBRLK1 |
| At5g06270 | B-type cyclin-related similar to B-type cyclin GI: 849074 from [Nicotiana |
| | tabacum] |
| At5g65070 | MADS-box protein |
| At5g01780 | oxidoreductase, 2OG-Fe(II) oxygenase family low similarity to alkB protein - |
| | Escherichia coli, PIR: BVECKB, alkB [Caulobacter crescentus][GI: 2055386]; |
| | contains Pfam domain PF03171 2OG-Fe(II) oxygenase superfamily |
| At5g45600 | expressed protein contains similarity to unknown protein (gb |
| At5g15370 | hypothetical protein |
| At5g11290 | hypothetical protein predicted proteins, Arabidopsis thaliana |
| At5g42910 | ABA-responsive element binding protein, putative |
| At5g10410 | expressed protein putative protein, Arabidopsis thaliana |
| At5g39500 | pattern formation protein, putative similar to SP |
| At1g60300 | hypothetical protein contains similarity to jasmonic acid 2 GI: 6175246 from |
| | [Lycopersicon esculentum] |
| At4g34060 | hypothetical protein |
| At3g42480 | hypothetical protein hypothetical proteins - Arabidopsis thaliana |
| At4g24530 | PsRT17-1 like protein PsRT17-1, Pisum sativum (pea), PATX: G1778376 |
| At2g32590 | hypothetical protein |
| At2g27280 | hypothetical protein |
| At1g12190 | F-box protein family contains F-box domain Pfam: PF00646 |
| At1g22720 | WAK-like kinase (WLK) contains similarity to serine/threonine kinase gb |
| At4g04400 | hypothetical protein contains Pfam profile PF03384: Drosophila protein of |
| | unknown function, DUF287 |
| At2g46740 | FAD-linked oxidoreductase family strong similarity to At1g32300, At5g56490, |
| | At2g46750, At2g46760; contains PF01565: FAD binding domain |
| At1g62630 | disease resistance protein (CC-NBS-LRR class), putative domain signature |
| | CC-NBS-LRR exists, suggestive of a disease resistance protein. |
| At2g13900 | CHP-rich zinc finger protein, putative |
| At4g28630 | ABC transporter family protein identical to half-molecule ABC transporter ATM1 |
| | GI: 9964117 from [Arabidopsis thaliana] |
| AtCg00320 | trnfM: tRNA-Phe |
| At1g31320 | lateral organ boundaries (LOB) domain family similar to lateral organ |
| | boundaries (LOB) domain-containing proteins from Arabidopsis thaliana |
| At1g24200 | hypothetical protein similar to hypothetical protein, GB: AAB61107 |
| At1g04070 | expressed protein Contains similarity to hypothetical mitochondrial import |
| | receptor subunit gb Z98597 from S. pombe. ESTs gb |
| At1g53760 | hypothetical protein |
| At1g72810 | threonine synthase, putative strong similarity to SP |
| At1g10522 | expressed protein |
| At1g78100 | F-box protein family contains F-box domain Pfam: PF00646 |
| At1g72410 | hypothetical protein similar to N-term of COP1-Interacting Protein 7 |
| | GB: BAA31739 [Arabidopsis thaliana] |
| At1g68720 | deaminase-related similar to cytidine/deoxycytidylate deaminase family protein |
| | GB: AAF73539 GI: 8163170 from [Chlamydia muridarum] |
| At1g34300 | expressed protein contains similarity to receptor-like protein kinase GI: 6979335 |
| | from [Oryza sativa] |
| At1g27850 | transposon protein-related similar to En/Spm-like transposon protein |
| | GB: AAB95292 GI: 2088658 from [Arabidopsis thaliana] |
| At1g11220 | expressed protein contains similarity to cotton fiber expressed protein |
| | GB: AAC33276 from [Gossypium hirsutum] |
| At1g73970 | expressed protein |
| At1g66840 | hypothetical protein |
| At1g01650 | expressed protein |
| At2g26310 | expressed protein and grail |
| At2g27490 | expressed protein |
| At2g22290 | GTP-binding protein, putative similar to GTP-binding protein GI: 550072 from |
| | [Homo sapiens] |
| At2g45280 | RAD51C DNA repair protein-related |
| At3g05460 | expressed protein |
| At3g24230 | polysaccharide lyase family 1 (pectate lyase) similar to pectate lyase |
| | GP: 14531296 from [Fragaria x ananassa] |
| At3g04605 | Mutator-related transposase similar to MURA transposase of maize Mutator |
| | transposon |
| At3g52900 | expressed protein chromosome assembly protein homolog, Aquifex aeolicus, |
| | PIR: B70356 |
| At3g08000 | RNA-binding protein, putative similar to RNA-binding protein from [Nicotiana |
| | tabacum] GI: 15822703, [Nicotiana sylvestris] GI: 624925; contains Pfam |
| | profile: PF00076 RNA recognition motif. (a.k.a. RRM, RBD, or RNP domain) |
| At3g15120 | chaperone-related ATPase contains Pfam profile: PF00004 ATPases |
| | associated with various cellular activities (AAA) |
| At3g45100 | n-acetylglucosaminyl-phosphatidylinositol biosynthetic protein, putative similar |
| | to PIG-A from Mus musculus [gi: 577723[, Homo sapiens [SP |
| At4g04360 | hypothetical protein |
| At4g26850 | expressed protein |
| At4g35280 | zinc-finger protein-related PEThy; ZPT4-1, Petunia x hybrida |
| At2g43970 | VirF-interacting protein FIP1 |
| At4g33180 | hydrolase, alpha/beta fold family low similarity to 2-hydroxy-6-oxo-7- |
| | methylocta-2,4-dienoate hydrolase [Pseudomonas fluorescens] GI: 1871461; |
| | contains Pfam profile PF00561: alpha/beta hydrolase fold |
| At5g10620 | hypothetical protein predicted protein, Bacillus subtilis |
| At3g25725 | pseudogene, similar to open reading frame 1 (Ty1_Copia-element) [Brassica |
| | oleracea] (GB: CAA72989) |
| At5g65480 | expressed protein similar to unknown protein (pir |
| At5g44870 | disease resistance protein (TIR-NBS-LRR class), putative domain signature |
| | TIR-NBS-LRR exists, suggestive of a disease resistance protein. |
| At5g47550 | expressed protein similar to unknown protein (pir |
| At5g39360 | expressed protein predicted proteins, Arabidopsis thaliana |
| At3g23570 | expressed protein contains Pfam profile: PF01738 dienelactone hydrolase |
| | family |
| At1g74910 | ADP-glucose pyrophosphorylase family contains Pfam profile PF00483: |
| | Nucleotidyl transferase; low similarity to mannose-1-phosphate |
| | guanylyltransferase [Hypocrea jecorina] GI: 3323397 |
| At3g19980 | protein phosphatase similar to serine/threonine protein phosphatase |
| | GB: Z47076 GI: 1143510 [Malus domestica] |
| At3g42220 | transposase - like protein putative transposase protein Shooter, Zea mays, |
| | EMBL: AF136220 |
| At4g06718 | pseudogene, predicted protein |
| At2g29900 | presenilin-related |
| At3g05110 | hypothetical protein |
| At3g10270 | DNA topoisomerase [ATP-hydrolyzing] (DNA topoisomerase II/DNA gyrase), |
| | putative similar to SP |
| At1g24090 | RNase H domain-containing protein very low similarity to GAG-POL precursor |
| | [Oryza sativa (japonica cultivar-group)] GI: 5902445; contains Pfam profiles |
| | PF00075: RNase H, PF04134: Protein of unknown function, DUF393 |
| AtCg00570 | psbF: cytochrome b559 beta chain |
| At1g06020 | pfkB type carbohydrate kinase protein family similar to fructokinase GI: 2102693 |
| | from [Lycopersicon esculentum] |
| At1g03580 | hypothetical protein temporary automated functional assignment |
| At1g49110 | hypothetical protein |
| At1g08260 | DNA polymerase epsilon catalytic subunit-related similar to DNA polymerase |
| | epsilon catalytic subunit GI: 5565875 from [Mus musculus] |
| At1g69970 | CLE26, putative CLAVATA3/ESR-Related 26 (CLE26); |
| At1g14360 | expressed protein |
| At1g24270 | hypothetical protein |
| At1g30190 | hypothetical protein |
| At1g02650 | DnaJ domain-containing protein contains Pfam profile PF00226: DnaJ domain |
| At4g21680 | peptide transporter - like protein peptide transporter (ptr1) - Hordeum |
| | vulgare, AF023472 |
| At5g55540 | expressed protein similar to unknown protein (gb |
| At2g33550 | expressed protein |
| At2g28520 | vacuolar proton-ATPase subunit-related |
| At2g46250 | expressed protein and genefinder |
| At2g37650 | scarecrow transcription factor family |
| At2g42230 | expressed protein |
| At2g34190 | membrane transporter-related |
| At3g43180 | C3HC4-type zinc finger protein family contains Pfam profile: PF00097 zinc |
| | finger, C3HC4 type (RING finger) |
| At3g52620 | hypothetical protein phosphate actyltransferase, Staphylococcus aureus, |
| | EMBL: SAU271496 |
| At3g06580 | galactokinase identical to galactokinase (Galactose kinase) [Arabidopsis |
| | thaliana] SWISS-PROT: Q9SEE5 |
| At3g27390 | expressed protein |
| At3g12550 | expressed protein |
| At3g58710 | WRKY family transcription factor contains Pfam profile: PF03106 WRKY DNA - |
| | binding domain |
| At3g62980 | transport inhibitor response 1 (TIR1), AtFBL1 E3 ubiquitin ligase SCF complex |
| | F-box subunit; identical to transport inhibitor response 1 GI: 2352492 from |
| | [Arabidopsis thaliana] |
| At3g03190 | glutathione transferase, putative identical to glutathione S-transferase |
| | GB: AAB09584 from [Arabidopsis thaliana] |
| At3g09950 | hypothetical protein |
| At3g21230 | 4-coumarate: CoA ligase (4-coumaroyl-CoA synthase) (4CL), putative similar to |
| | 4CL2 [gi: 12229665] and 4CL1 [gi: 12229649] from [Arabidopsis thaliana], 4CL1 |
| | [gi: 12229631] from Nicotiana tabacum |
| At4g13540 | expressed protein predicted protein, Arabidopsis thaliana |
| At4g29270 | acid phosphatase-related protein acid phosphatase-1 (EC 3.1.3.—) - |
| | Lycopersicon esculentum, PIR2: T06587 |
| At4g22570 | adenine phosphoribosyltransferase (EC 2.4.2.7) - like protein adenine |
| | phosphoribosyltransferase, Triticum aestivum, T06263 |
| At4g25430 | hypothetical protein |
| At5g12870 | myb family transcription factor contains PFAM profile: myb DNA binding |
| | domain PF00249 |
| At5g45170 | expressed protein similar to unknown protein (pir |
| At5g18260 | expressed protein |
| At5g01720 | F-box protein family (FBL3) contains similarity to leucine-rich repeats |
| | containing F-box protein FBL3 GI: 5919219 from [Homo sapiens] |
| At5g01900 | WRKY family transcription factor contains Pfam profile: PF03106 WRKY DNA |
| | binding domain |
| At5g39380 | expressed protein predicted protein, Arabidopsis thaliana |
| At5g66560 | phototropic response protein family contains NPH3 family domain, |
| | Pfam: PF03000 |
| At5g18070 | N-acetylglucosamine-phosphate mutase |
| At5g41850 | hypothetical protein |
| At5g15470 | glycosyltransferase family 8 contains Pfam profile: PF01501 glycosyl |
| | transferase family 8 |
| At5g38960 | germin-like protein, putative similar to germin-like protein subfamily 1 member |
| | 8 [SP |
| At5g41460 | fringe-related protein strong similarity to unknown protein (pir |
| At5g62070 | expressed protein various predicted proteins, Arabidopsis thaliana |
| At1g73010 | expressed protein |
| At1g31970 | DEAD/DEAH box helicase, putative similar to p68 RNA helicase |
| | [Schizosaccharomyces pombe] GI: 173419 |
| At5g49160 | DNA (cytosine-5)-methyltransferase (DNA methyltransferase) (DNA metase) |
| | (sp |
| At3g46430 | expressed protein mitochondrial ATP SYNTHASE 6 KD SUBUNIT - Solanum |
| | tuberosum, SWISSPROT: P80497 |
| At3g46170 | short-chain dehydrogenase/reductase family protein contains similarity to 3- |
| | oxoacyl-[acyl-carrier protein] reductase SP: P51831 from [Bacillus subtilis] |
| At4g05580 | contains similarity to Arabidopsis thaliana hypothetical protein (GB: AL022580) |
| At4g22070 | WRKY family transcription factor identical to WRKY transcription factor 31 |
| | (WRKY31) GI: 15990589 from [Arabidopsis thaliana] |
| At5g06390 | expressed protein strong similarity to unknown protein (gb |
| At2g32430 | galactosyltransferase family contains Pfam profile: PF01762 |
| | galactosyltransferase |
| At1g71240 | expressed protein |
| At1g37080 | hypothetical protein |
| At3g23980 | expressed protein |
| At1g03060 | putataive transport protein Similar to gb |
| At4g34090 | expressed protein |
| At1g55740 | glycosyl hydrolase family 36 similar to seed imbibition protein GB: AAA32975 |
| | GI: 167100 from [Hordeum vulgare] |
| At1g16190 | DNA repair protein RAD23, putative similar to DNA repair by nucleotide |
| | excision (NER) RAD23 protein, isoform II GI: 1914685 from [Daucus carota] |
| At1g59540 | kinesin-related protein similar to kinesin motor protein (kin2) GI: 2062751 from |
| | (Ustilago maydis) |
| At1g29520 | plasma membrane associated protein-related similar to GI: 6851373 from |
| | [Hordeum vulgare] |
| At1g70540 | expressed protein contains Pfam profile PF04043: Plant invertase/pectin |
| | methylesterase inhibitor |
| At1g12030 | hypothetical protein |
| At1g01810 | hypothetical protein |
| At1g75200 | flavodoxin family contains Pfam profiles PF00258: Flavodoxin, PF04055: |
| | radical SAM domain protein |
| At1g64355 | expressed protein |
| At1g47350 | hypothetical protein similar to hypothetical protein GB: AAD22292 GI: 6598654 |
| | from [Arabidopsis thaliana] |
| At5g66020 | hypothetical protein non-consensus AT donor splice site at exon 7, TA donor |
| | splice site at exon 10, AT acceptor splice at exon 13, strong similarity to |
| | unknown protein (emb |
| At2g46710 | rac GTPase activating protein-related |
| At2g33560 | expressed protein |
| At2g15790 | cyclophilin-40 annotation temporarily based on supporting cDNA gi |
| At2g02780 | leucine-rich repeat transmembrane protein kinase, putative |
| At2g43420 | 3-beta hydroxysteroid dehydrogenase/isomerase family contains Pfam profile |
| | PF01073 3-beta hydroxysteroid dehydrogenase/isomerase domain; similar to |
| | NAD(P)-dependent steroid dehydrogenase from Homo sapiens [SP |
| At2g20980 | hypothetical protein and genefinder |
| At3g56980 | bHLH protein family NULL |
| At3g52200 | dihydrolipoamide S-acetyltransferase (LTA3); nuclear gene encoding |
| | mitochondrial protein annotation temporarily based on supporting cDNA gi |
| At3g10400 | RNA recognition motif (RRM) - containing protein low similarity to splicing |
| | factor SC35 [Arabidopsis thaliana] GI: 9843653; contains InterPro entry |
| | IPR000504: RNA-binding region RNP-1 (RNA recognition motif) (RRM) |
| At3g25960 | pyruvate kinase, putative similar to pyruvate kinase, cytosolic isozyme |
| | [Nicotiana tabacum] SWISS-PROT: Q42954 |
| At3g44680 | expressed protein histone deacetylase 1 - Gallus gallus, EMBL: AF043328 |
| At3g13682 | amine oxidase family similar to polyamine oxidase isoform-1 [Homo sapiens] |
| | GI: 14860862; contains Pfam profile: PF01593 Flavin containing amine oxidase |
| At3g07990 | serine carboxypeptidase-related similar to serine carboxypeptidase II (CP-MII) |
| | GB: CAA70815 [Hordeum vulgare] |
| At3g06270 | protein phosphatase 2C (PP2C), putative similar to protein phosphatase-2C |
| | (PP2C) GB: AAC36699 [Mesembryanthemum crystallinum]; contains Pfam |
| | profile: PF00481 protein phosphatase 2C |
| At3g05200 | RING-H2 zinc finger protein ATL6-related similar to GB: AAD33584 from |
| | [Arabidopsis thaliana] |
| At3g48610 | phosphoesterase family low similarity to SP |
| At3g61600 | POZ domain protein family contains Pfam PF00651: BTB/POZ domain; |
| | contains Interpro IPR000210/PS50097: BTBB/POZ domain; similar to |
| | POZ/BTB containing-protein AtPOB1 (GI: 12006855) [Arabidopsis thaliana]; |
| | similar to actinfilin (GI: 21667852) [Rattus norv? |
| At4g21910 | MATE efflux protein family similar to ripening regulated protein DDTFR18 |
| | [Lycopersicon esculentum] GI: 12231296; contains Pfam profile PF01554: |
| | Uncharacterized membrane protein family |
| At4g36030 | armadillo repeat containing protein |
| At4g32120 | galactosyltransferase family contains Pfam profile: PF01762 |
| | galactosyltransferase |
| At4g13420 | potassium transporter, putative (HAK5/POT5) identical to K+ transporter HAK5 |
| | [Arabidopsis thaliana] gi |
| At4g10300 | expressed protein predicted protein, Arabidopsis thaliana |
| At5g62030 | expressed protein predicted proteins, D. melanogaster, C. elegans and yeast |
| At5g37790 | protein kinase family contains protein kinase domain, Pfam: PF00069 |
| At5g61850 | LFY floral meristem identity control protein |
| At5g44040 | expressed protein similar to unknown protein (gb |
| At5g40580 | 20S proteasome beta subunit B (PBB2) |
| At4g33800 | expressed protein |
| At3g15354 | WD-40 repeat protein family contains 7 WD-40 repeats (PF00400); |
| | phytochrome A supressor spa1 (GI: 4809171) [Arabidopsis thaliana] |
| At4g16070 | lipase (class 3) family low similarity to calmodulin-binding heat-shock protein |
| | CaMBP [Nicotiana tabacum] GI: 1087073; contains Pfam profile PF01764: |
| | Lipase, PF03893: Lipase 3 N-terminal region |
| At5g41060 | DHHC-type zinc finger domain-containing protein contains Pfam profile |
| | PF01529: DHHC zinc finger domain |
| At1g52070 | jacalin lectin family similar to myrosinase-binding protein homolog [Arabidopsis |
| | thaliana] GI: 2997767; contains Pfam profile PF01419 jacalin-like lectin domain |
| At4g24260 | glycosyl hydrolase family 9 (endo-1,4-beta-glucanase) similar to endo-1,4- |
| | beta-D-glucanase; cellulase GI: 5689613 from [Brassica napus] |
| At4g11200 | hypothetical protein other hypothetical proteins Arabidopsis thaliana |
| At3g45940 | glycosyl hydrolase family 31 similar to alpha-xylosidase precursor GI: 4163997 |
| | from [Arabidopsis thaliana] |
| At5g66710 | protein kinase, putative similar to protein kinase ATN1 GP |
| At2g45900 | expressed protein |
| At2g17160 | hypothetical protein identical to hypothetical protein GB: AAB81676 |
| At1g68280 | hypothetical protein |
| At1g19580 | transferase hexapeptide repeat family contains Pfam profile PF00132: Bacterial |
| | transferase hexapeptide (four repeats) |
| At3g13830 | F-box protein family contains Pfam: PF00646 F-box domain; contains |
| | TIGRFAM TIGR01640: F-box protein interaction domain |
| At4g01730 | DHHC-type zinc finger domain-containing protein contains Pfam profile |
| | PF01529: DHHC zinc finger domain |
| At3g11010 | disease resistance protein family (LRR) contains leucine rich-repeat domains |
| | Pfam: PF00560, INTERPRO: IPR001611; similar to disease resistance protein |
| | [Lycopersicon esculentum] gi |
| At2g26740 | epoxide hydrolase (ATsEH) identical to ATsEH [Arabidopsis thaliana] |
| | GI: 1109600 |
| At1g23460 | polygalacturonase, putative similar to polygalacturonase GB: BAA88472 |
| | GI: 6624205 from (Cucumis sativus) |
| | trnV&trnM |
| At1g48310 | SNF2domain/helicase domain-containing protein contains similarity to DNA- |
| | dependent ATPase A GI: 6651385 from [Bos taurus]}; contains PFam profiles |
| | PF00271: Helicase conserved C-terminal domain, PF00176: SNF2 |
| At1g21620 | pumilio-family RNA-binding protein, putative similar to hypothetical protein |
| | GB: AAD41414 GI: 5263312 from (Arabidopsis thaliana) |
| At1g13630 | pentatricopeptide (PPR) repeat-containing protein contains Pfam profile: |
| | PF01535 PPR repeat |
| At1g16640 | transcriptional factor B3 family low similarity to reproductive meristem protein 1 |
| | [Arabidopsis thaliana] GI: 13604227; contains Pfam profile PF02362: B3 DNA |
| | binding domain |
| At1g18460 | lipase family similar to triacylglycerol lipase, gastric precursor (EC 3.1.1.3) |
| | {Canis familiaris} [SP |
| At1g28560 | expressed protein |
| At1g49890 | expressed protein |
| At1g32190 | expressed protein similar to hypothetical protein GB: AAD18105 GI: 4337191 |
| | from [Arabidopsis thaliana] |
| At1g06560 | expressed protein |
| At4g08680 | MuDR-A transposon protein-related similar to Z. mays MuDR-A protein |
| At5g41490 | hypothetical protein strong similarity to unknown protein (gb |
| At2g02790 | hypothetical protein |
| At2g39870 | expressed protein |
| At2g41040 | expressed protein |
| At2g15050 | lipid transfer protein, putative similar to SP |
| At2g16620 | protein kinase-related contains a protein kinase domain profile (PDOC00100) |
| At2g28250 | protein kinase family contains protein kinase domain, Pfam: PF00069 |
| At3g55560 | expressed protein AT-hook protein 1 (AHP1), Arabidopsis thaliana, |
| | EMBL: ATAHP1 |
| At3g20720 | expressed protein |
| At3g01900 | cytochrome P450 family similar to Cytochrome P450 94A1 (P450-dependent |
| | fatty acid omega-hydroxylase) (SP: O81117) {Vicia sativa}; contains Pfam |
| | profile: PF00067 cytochrome P450 |
| At3g62420 | bZIP family transcription factor similar to common plant regulatory factor 6 |
| | GI: 9650826 from [Petroselinum crispum] |
| At3g20030 | F-box protein family contains F-box domain Pfam: PF00646 |
| At3g58320 | hypothetical protein several hypothetical proteins - Arabidopsis thaliana |
| At4g30860 | SET-domain transcriptional regulator family low similarity to IL-5 promoter REII- |
| | region-binding protein [Homo sapiens] GI: 12642795; contains Pfam profile |
| | PF00856: SET domain |
| At4g33840 | glycosyl hydrolase family 10 xylan endohydrolase isoenzyme X-I, Hordeum |
| | vulgare, PID: g1813595 |
| At4g29310 | expressed protein hypothetical protein T27I1.4 - Arabidopsis |
| | thaliana, PID: g3540181 |
| At4g22170 | F-box protein family contains F-box domain Pfam: PF00646 |
| At4g30680 | MA3 domain-containing protein similar to SP |
| At4g26660 | expressed protein probable kinesin - Arabidopsis thaliana, Pir2: H71402 |
| At4g25510 | hypothetical protein |
| At4g33400 | Dem-related protein Dem (defective embryo and meristems) protein - |
| | Lycopersicon esculentum, PID: e321604 |
| At4g27980 | expressed protein |
| At4g39050 | kinesin-related protein kinesin motor protein - Ustilago maydis, PID: g2062750 |
| At4g24050 | short-chain dehydrogenase/reductase family protein contains INTERPRO |
| | family IPR002198 Short-chain dehydrogenase/reductase (SDR) superfamily |
| At4g37710 | expressed protein predicted protein, Arabidopsis thaliana |
| At5g42320 | hypothetical protein |
| At5g64190 | expressed protein strong similarity to unknown protein (gb |
| At5g51210 | oleosin |
| At5g03180 | C3HC4-type zinc finger protein family various predicted proteins, Arabidopsis |
| | thaliana; contains Pfam profile PF00097: Zinc finger, C3HC4 type (RING |
| | finger) |
| At5g65310 | homeobox-leucine zipper protein ATHB-5 (HD-Zip protein ATHB-5) identical to |
| | homeobox-leucine zipper protein ATHB-5 (HD-ZIP protein ATHB-5) |
| | (SP: P46667) [Arabidopsis thaliana] |
| At5g59940 | CHP-rich zinc finger protein, putative large number of predicted zinc finger |
| | proteins, Arabidopsis thaliana, Homo sapiens and others |
| At5g56120 | expressed protein similar to unknown protein (dbj |
| At2g03820 | nonsense-mediated mRNA decay protein-related |
| At1g61820 | glycosyl hydrolase family 1 similar to beta-glucosidase GI: 1155254 from |
| | [Prunus avium] |
| At5g44750 | expressed protein contains similarity to DNA-damage-inducible protein P |
| At2g23400 | dehydrodolichyl diphosphate synthase [DEDOL-PP synthase], putative similar |
| | to GI: 796076 |
| At4g11720 | hypothetical protein histidine-rich glycoprotein precursor, Plasmodium |
| | lophurae, PIR1: KGZQHL |
| At3g26250 | CHP-rich zinc finger protein, putative |
| At1g23150 | expressed protein location of EST gb |
| At2g24950 | hypothetical protein contains Pfam profile PF03080: Arabidopsis proteins of |
| | unknown function |
| At3g14517 | pseudogene, similar to L1 repeat, Tf subfamily, member 30 (LINE-element) |
| | [Mus musculus] (GB: NP_038605) |
| At3g16390 | jacalin lectin family similar to myrosinase-binding protein homolog [Arabidopsis |
| | thaliana] GI: 2997767, epithiospecifier [Arabidopsis thaliana] GI: 16118845; |
| | contains Pfam profiles PF01419 jacalin-like lectin family, PF01344 Kelch motif |
| At2g35330 | expressed protein |
| At1g56110 | nucleolar protein Nop56, putative similar to XNop56 protein [Xenopus laevis] |
| | GI: 14799394; contains Pfam profile PF01798: Putative snoRNA binding |
| | domain |
| At5g66910 | disease resistance protein (CC-NBS-LRR class), putative domain signature |
| | CC-NBS-LRR exists, suggestive of a disease resistance protein. |
| At2g17200 | ubiquitin protein-related |
| AtCg00580 | psbE: cytochrome b559 alpha chain |
| At1g47840 | hexokinase-related similar to hexokinase 2 GB: AAB49911 GI: 1899025 from |
| | [Arabidopsis thaliana] |
| At1g28430 | cytochrome P450, putative similar to cytochrome P450 (CYP93A1) GI: 1435059 |
| | from [Glycine max] |
| At1g65870 | disease resistance response protein-related/dirigent protein-related similar to |
| | dirigent protein [Forsythia x intermedia] gi |
| At1g53280 | expressed protein similar to DJ-1 protein [Homo sapiens] GI: 1780755; contains |
| | Pfam profile: PF01965 ThiJ/PfpI family |
| At1g55880 | pyridoxal-5′-phosphate-dependent enzyme, beta family similar to SP |
| At1g57780 | heavy-metal-associated domain-containing protein low similarity to myosin-like |
| | antigen GI: 159877 Onchocerca volvulus; contains Pfam profile PF00403: |
| | Heavy-metal-associated domain |
| At1g17250 | leucine rich repeat protein family contains leucine rich-repeat (LRR) domains |
| | Pfam: PF00560, INTERPRO: IPR001611; similar to Hcr2-0B [Lycopersicon |
| | esculentum] gi |
| At5g21070 | expressed protein predicted protein - Oryza sativa - TREMBL: AP001072_3 |
| At2g38780 | expressed protein |
| At2g13840 | expressed protein |
| At2g48100 | exonuclease-related annotation temporarily based on supporting cDNA gi |
| At5g34960 | hypothetical protein common family includes At5g34960, At2g14450, |
| | At1g35920 |
| At2g37040 | phenylalanine ammonia lyase (PAL1) nearly identical to SP |
| At3g55910 | expressed protein PA26, p53 regulated PA26-T3 nuclear protein, Homo |
| | sapiens, EMBL: AF033121 |
| At3g11490 | rac GTPase activating protein-related similar to rac GTPase activating protein |
| | 1 GB: AAC62624 [Lotus japonicus] |
| At3g04460 | expressed protein similar to peroxisomal biogenesis factor 12 GB: NP_000277 |
| | [Homo sapiens] |
| At3g13140 | hypothetical protein |
| At3g04800 | inner mitochondrial membrane protein-related similar to inner mitochondrial |
| | membrane protein GB: S71194 (Arabidopsis thaliana) |
| At3g23420 | hypothetical protein |
| At3g43720 | protease inhibitor/seed storage/lipid transfer protein (LTP) family contains Pfam |
| | protease inhibitor/seed storage/LTP family domain PF00234 |
| At4g39220 | AtRer1A |
| At4g12240 | hypothetical proteins |
| At4g07770 | pseudogene, similar to L1 repeat, Tf subfamily, member 30 (LINE-element) |
| | [Mus musculus] (GB: NP_038605) |
| At4g14920 | PHD finger transcription factor, putative |
| At4g22880 | leucoanthocyanidin dioxygenase (anthocyanidin synthase) (LDOX/ANS), |
| | putative similar to SP |
| At4g18670 | leucine-rich repeat extensin family similar to extensin-like protein [Lycopersicon |
| | esculentum] gi |
| At5g46280 | DNA replication licensing factor, putative similar to SP |
| At5g15340 | pentatricopeptide (PPR) repeat-containing protein contains |
| | INTERPRO: IPR002885 PPR repeats |
| At5g20070 | MutT/nudix family protein low similarity to SP |
| At5g46570 | protein kinase family contains protein kinase domain, Pfam: PF00069 |
| At5g43920 | transducin/WD-40 repeat protein family contains 7 WD-40 repeats (PF00400); |
| | similar to will die slowly protein (WDS) (SP: Q9V3J8) [Drosophila |
| | melanogaster] |
| At5g16890 | Exostosin family contains Pfam profile: PF03016 Exostosin family |
| At5g43340 | inorganic phosphate transporter identical to inorganic phosphate transporter |
| | [Arabidopsis thaliana] GI: 3869190 |
| At5g02250 | ribonuclease II-related protein ribonuclease II family protein, Deinococcus |
| | radiodurans, PIR: C75571 |
| At5g42250 | alcohol dehydrogenase (ADH), putative similar to alcohol dehydrogenase ADH |
| | GI: 7705214 from [Lycopersicon esculentum]; contains Pfam zinc-binding |
| | dehydrogenase domain PF00107 |
| At5g58540 | expressed protein serine/threonine-specific protein kinase NPK15, Nicotiana |
| | tabacum, PIR: S52578 |
| At5g52510 | scarecrow-like transcription factor 8 (SCL8) |
| At5g13800 | hydrolase, alpha/beta fold family low similarity to hydrolase [Terrabacter sp. |
| | DBF63] GI: 14196240; contains Pfam profile PF00561: hydrolase, alpha/beta |
| | fold family |
| At5g51640 | (YLS7) leaf-senescence-related protein annotation temporarily based on |
| | supporting cDNA gi |
| At5g04770 | amino acid transport - like protein amino acid transport protein AAT1, |
| | Arabidopsis thaliana, PIR: S51171 |
| At2g43640 | signal recognition particle protein 14 kD, ATSRP14-related |
| At4g11090 | expressed protein other hypothetical proteins - Arabidopsis thaliana |
| At4g14990 | expressed protein |
| At4g03870 | pseudogene, putative transposon protein similar to MuDR transposon |
| At5g49270 | expressed protein contains similarity to phytochelatin synthetase |
| At5g61110 | hypothetical protein |
| At5g07810 | SNF2 domain/helicase domain-containing protein similar to HepA-related |
| | protein HARP [Homo sapiens] GI: 6693791; contains Pfam profiles PF00271: |
| | Helicase conserved C-terminal domain, PF00176: SNF2 family N-terminal |
| | domain, PF01844: HNH endonuclease |
| At2g32920 | protein disulfide isomerase family similar to SP |
| At2g29920 | hypothetical protein |
| At1g37045 | an Arabidopsis thaliana hypothetical protein, which contains similarity to |
| | retrotransposon Athila (GB: AF076275)-related temporary automated |
| | functional assignment |
| At3g10660 | calcium-dependent protein kinase (CDPK)(CPK2) identical to calcium- |
| | dependent protein kinase isoform 2 [Arabidopsis thaliana] gi |
| At5g59920 | CHP-rich zinc finger protein, putative large number of predicted zinc finger |
| | proteins, Arabidopsis thaliana, Homo sapiens and others |
| At1g65385 | pseudogene, putative serpin |
| At5g61700 | ABC transporter family protein ABC family transporter, Entamoeba histolytica, |
| | EMBL: EH058 |
| AtCg00260 | trnT.1: tRNA-Thr |
| At1g56720 | protein kinase family contains protein kinase domain, Pfam: PF00069 |
| At4g06620 | pseudogene, similar to polyprotein (Gypsy_Ty3-element) [Ananas comosus] |
| | (GB: CAA73042) |
| At1g49160 | protein kinase family contains protein kinase domain, Pfam: PF00069 |
| At1g78980 | leucine-rich repeat transmembrane protein kinase, putative similar to leucine- |
| | rich repeat transmembrane protein kinase 2 GI: 3360291 from [Zea mays] |
| At1g02770 | expressed protein similar to Hypothetical protein GB: AAF02890 GI: 6056426 |
| | from (Arabidopsis thaliana) |
| At1g66720 | methyltransferase-related similar to defense-related protein cjs1 [Brassica |
| | carinata][GI: 14009292][Mol Plant Pathol (2001) 2(3): 159-169] |
| At2g40475 | expressed protein |
| At5g19240 | expressed protein predicted protein, Arabidopsis thaliana |
| At5g64960 | Cyclin-dependent kinase C; 2 |
| At1g35460 | bHLH protein similar to GI: 6166283 from [Pinus taeda] |
| At2g17870 | glycine-rich, zinc-finger DNA-binding protein-related genomic copy of EST |
| | T76328 cold-shock signature from position 22 to 41 |
| | [YGFITPDDGGEELFVHQSSI]; 7 copies of CCHC zinc-finger motif, from 94 to |
| | 107 [CFNCGEVGHMAKDC], from 130 to 142 |
| At2g44080 | expressed protein |
| At2g30490 | cytochrome P450 73/trans-cinnamate 4-monooxygenase/cinnamate-4- |
| | hydroxylase (CYP73) (C4H) identical to SP |
| At2g19590 | 1-aminocyclopropane-1-carboxylate oxidase (ACC oxidase), putative similar to |
| | ACC oxidase [Cucumis melo][GI: 1183898] |
| At2g42070 | MutT/nudix family protein similar to SP |
| At3g50170 | hypothetical protein various predicted genes, Arabidopsis thaliana and Oryza |
| | sativa |
| At3g59870 | expressed protein hypothetical protein F6E13.7 - Arabidopsis thaliana, |
| | PIR: T00674 |
| At3g54680 | proteophosphoglycan-related contains similarity to proteophosphoglycan |
| | [Leishmania major] gi |
| At3g61270 | expressed protein several hypothetical proteins - Arabidopsis thaliana |
| At3g03120 | ADP-ribosylation factor, putative similar to ADP-ribosylation factor 1; ARF 1 |
| | (GP: 385340) {Drosophila melanogaster} |
| At3g25190 | integral membrane protein-related contains Pfam profile: PF01988 integral |
| | membrane protein; similar to nodulin-21 GB: CAA34506 [Glycine max] |
| At3g02290 | C3HC4-type zinc finger protein family contains zinc finger motif, C3HC4 type |
| | (RING finger) |
| At3g14500 | hypothetical protein |
| At3g29200 | chorismate mutase, chloroplast (CM1) identical to chorismate mutase |
| | GB: Z26519 [SP |
| At3g22050 | hypothetical protein contains Pfam profile: PF01657 Domain of unknown |
| | function |
| At4g12700 | expressed protein |
| At4g24230 | expressed protein acyl-CoA binding protein - Arabidopsis |
| | thaliana, PID: g4128197 |
| At4g27340 | expressed protein met-10+ protein, Neurospora crassa, PIR2: S46697 |
| At4g38225 | expressed protein |
| At4g31280 | hypothetical protein |
| At4g23880 | hypothetical protein |
| At5g50280 | pentatricopeptide (PPR) repeat-containing protein contains Pfam profile |
| | PF01535: PPR repeat |
| At5g66150 | glycosyl hydrolase family 38 (alpha-mannosidase) similar to lysosomal alpha- |
| | mannosidase SP: O09159 from [Mus musculus] |
| At5g53940 | zinc-binding protein-related |
| At1g16400 | cytochrome P450 family similar to gb |
| At2g26750 | epoxide hydrolase, putative strong similarity to ATsEH [Arabidopsis thaliana] |
| | GI: 1109600 |
| At5g48290 | heavy-metal-associated domain-containing protein strong similarity to |
| | farnesylated proteins ATFP4 [GI: 4097549] and ATFP5 [GI: 4097551]; contains |
| | Pfam profile PF00403: Heavy-metal-associated domain |
| At1g21245 | wall-associated kinase 3-related temporary automated functional assignment |
| At5g60140 | transcriptional factor B3 family contains Pfam profile PF02362: B3 DNA binding |
| | domain |
| At3g44120 | F-box protein family contains Pfam: PF00646 F-box domain |
| AtCg00560 | psbL: photosystem II protein L |
| At1g03670 | hypothetical protein similar to hypothetical protein GB: Z97336 |
| At1g22090 | expressed protein |
| At1g04980 | protein disulfide isomerase family similar to SP |
| At1g04970 | expressed protein |
| At1g55750 | expressed protein |
| At1g59760 | ATP-dependent RNA helicase, putative similar to SP |
| At2g39300 | hypothetical protein |
| At2g37340 | splicing factor RSZ33, putative nearly identical to splicing factor RSZ33 |
| | [Arabidopsis thaliana] GI: 9843663 |
| At3g10210 | expressed protein similar to putative protein GB: CAA20045 [Arabidopsis |
| | thaliana] |
| At3g05430 | PWWP domain protein contains Pfam profile: PF00855 PWWP domain |
| At3g48600 | expressed protein |
| At3g62880 | expressed protein amino acid selective channel protein - Hordeum vulgare, |
| | EMBL: AJ011921 |
| At4g08280 | expressed protein hypothetical protein ssr1391 - Synechocystis sp. (strain PCC6803), |
| | PIR2: S75571 |
| At4g14830 | expressed protein |
| At5g19780 | tubulin alpha-3/alpha-5 chain (TUA5) nearly identical to SP |
| At5g55050 | GDSL-motif lipase/hydrolase protein similar to family II lipases EXL3 |
| | GI: 15054386, EXL1 GI: 15054382, EXL2 GI: 15054384 from [Arabidopsis |
| | thaliana]; contains Pfam profile PF00657: GDSL-like Lipase/Acylhydrolase |
| At5g39260 | expansin, putative (EXP21) similar to alpha-expansin GI: 6573157 from |
| | [Regnellidium diphyllum]; alpha-expansin gene family, PMID: 11641069 |
| At5g38350 | disease resistance protein (NBS-LRR class), putative domain signature NBS- |
| | LRR exists, suggestive of a disease resistance protein. |
| At5g51550 | expressed protein similar to unknown protein (gb |
| At4g39780 | AP2 domain transcription factor, putative similar to AP2 domain containing |
| | protein RAP2.4, Arabidopsis thaliana |
| At1g10650 | conserved hypothetical protein |
| At3g12430 | hypothetical protein |
| At4g16060 | expressed protein |
| At5g01540 | receptor lectin kinase, putative similar to receptor lectin kinase 3 [Arabidopsis |
| | thaliana] gi |
| At5g27210 | expressed protein seven transmembrane domain orphan receptor, Mus |
| | musculus, EMBL: AF051098 |
| At5g48410 | glutamate receptor family (GLR1.3) plant glutamate receptor family, |
| | PMID: 11379626 |
| At4g10510 | subtilisin-like serine protease contains similarity to subtilase; SP1 GI: 9957714 |
| | from [Oryza sativa] |
| At1g14800 | hypothetical protein |
| At1g24220 | hypothetical protein |
| At1g61260 | cotton fiber expressed protein-related similar to cotton fiber expressed protein |
| | 1 GI: 3264828 from [Gossypium hirsutum] |
| At1g80960 | expressed protein |
| At1g67370 | meiotic asynaptic mutant 1 identical to meiotic asynaptic mutant 1 [Arabidopsis |
| | thaliana] GI: 7939627; contains Pfam profiles PF02301: DNA-binding HORMA |
| | domain, PF04433: SWIRM domain |
| At1g64460 | phosphatidylinositol 3- and 4-kinase family contains Pfam profile PF00454: |
| | Phosphatidylinositol 3- and 4-kinase |
| At1g75980 | expressed protein |
| At1g33800 | expressed protein |
| At2g47490 | mitochondrial carrier protein family contains Pfam profile: PF00153 |
| | mitochondrial carrier protein |
| At2g27950 | expressed protein |
| At2g44830 | protein kinase putative similar to protein kinase PVPK-1 [Phaseolus vulgaris] |
| | SWISS-PROT: P15792 |
| At3g24700 | F-box protein family contains F-box domain Pfam: PF00646 |
| At3g46160 | protein kinase-related contains eukaryotic protein kinase domain, |
| | INTERPRO: IPR000719 |
| At3g49670 | leucine-rich repeat transmembrane protein kinase, putative CLAVATA1 |
| | receptor kinase, Arabidopsis thaliana, EMBL: ATU96879 |
| At3g43200 | pseudogene, putative protein predicted proteins, Arabidopsis thaliana |
| At3g10970 | haloacid dehalogenase-like hydrolase family low similarity to genetic modifier |
| | [Zea mays] GI: 10444400; contains InterPro accession IPR005834: Haloacid |
| | dehalogenase-like hydrolase |
| At3g51920 | calmodulin 9 identical to calmodulin 9 GI: 5825602 from [Arabidopsis thaliana] |
| At3g11320 | phosphate translocator-related low similarity to |
| | phosphoenolpyruvate/phosphate translocator precursor [Mesembryanthemum |
| | crystallinum] GI: 9295275, phosphate translocator [Nicotiana tabacum] |
| | GI: 403023; contains Pfam profile: PF00892 Integral membrane pro? |
| At4g27680 | expressed protein MSP1 protein, Saccharomyces cerevisia, PIR2: A49506 |
| At4g00670 | hypothetical protein |
| At4g31320 | auxin-induced (indole-3-acetic acid induced) protein, putative (SAUR_c) similar |
| | to auxin-induced protein TGSAUR22 (GI: 10185820) [Tulipa gesnerian]; |
| | similar to auxin-induced protein 15A (SP: P33081) from [Glycine max] |
| At4g28980 | cdk-activating kinase 1At identical to Cdk-activating kinase 1At [Arabidopsis |
| | thaliana] gi |
| At4g16690 | esterase/lipase/thioesterase family similar to ethylene-induced esterase [Citrus |
| | sinensis] GI: 14279437, polyneuridine aldehyde esterase [Rauvolfia |
| | serpentina] GI: 6651393, SP |
| At5g57730 | hypothetical protein |
| At5g09560 | KH domain protein various predicted RNA binding proteins, Arabidopsis |
| | thaliana |
| At5g59770 | expressed protein protein tyrosine phosphatase-like protein, PTPLB, Mus |
| | musculus, EMBL: AF169286 |
| At5g47290 | myb family transcription factor contains PFAM profile: PF00249 myb-like DNA |
| | binding domain |
| At5g52170 | homeodomain protein similar to Anthocyaninless2 (ANL2) (GP: 5702094) |
| | [Arabidopsis thaliana]; contains Pfam PF00046: Homeobox domain and Pfam |
| | PF01852: START domain |
| At4g03260 | leucine rich repeat protein family contains leucine rich repeat (LRR) domains, |
| | Pfam: PF00560 |
| At2g31570 | glutathione peroxidase, putative |
| At1g52060 | jacalin lectin family similar to myrosinase-binding protein homolog [Arabidopsis |
| | thaliana] GI: 2997767; contains Pfam profile PF01419 jacalin-like lectin |
| | domain |
| At4g15396 | cytochrome P450-related similar to Cytochrome P450 90C1 |
| | (ROTUNDIFOLIA3) (SP: Q9M066) [Arabidopsis thaliana]; contains Pfam |
| | profile: PF00067: Cytochrome P450 {Arabidopsis thaliana} |
| At2g26190 | expressed protein |
| At1g21310 | extensin family protein contains extensin-like region, Pfam: PF04554 |
| At2g19450 | diacylglycerol O-acyltransferase (acyl CoA: diacylglycerol acyltransferase) |
| | (DGAT) identical to gi: 5050913, gi: 6625553 |
| At2g16340 | hypothetical protein |
| At3g10860 | ubiquinol-cytochrome C reductase complex ubiquinone-binding protein (QP-C)- |
| | related similar to ubiquinol-cytochrome C reductase complex ubiquinone- |
| | binding protein (QP-C) GB: P46269 [Solanum tuberosum] |
| At1g05650 | polygalacturonase, putative similar to GB: AAC23398 |
| At1g49080 | pseudogene, putative transposon protein similar to Antirrhinum majus TNP2 |
| | protein gb |
| At3g23280 | auxin-regulated protein contains Pfam profile: PF00023 ankyrin repeat |
| At1g28300 | transcriptional factor B3 protein leafy cotyledon 2 nearly identical to LEAFY |
| | COTYLEDON 2 [Arabidopsis thaliana] GI: 15987516; contains Pfam profile |
| | PF02362: B3 DNA binding domain |
| At1g04640 | lipoyltransferase identical to GB: BAA78386 |
| At1g36310 | expressed protein |
| At1g47860 | reverse transcriptase-related low similarity to reverse transcriptase |
| | [Arabidopsis thaliana] GI: 976278; contains Pfam profiles PF00078: Reverse |
| | transcriptase (RNA-dependent DNA polymerase), PF00096: Zinc finger, C2H2 |
| | type, PF03727: Hexokinase |
| At1g61410 | expressed protein similar to putative double strand break repair protein |
| | GI: 9651817 from [Arabidopsis thaliana] |
| At1g13940 | expressed protein identical to hypothetical protein GB: AAD39280 GI: 5080770 |
| | from [Arabidopsis thaliana] |
| At1g65650 | expressed protein similar to ubiquitin C-terminal hydrolase-like protein |
| | GI: 9759113 from [Arabidopsis thaliana] |
| At1g31150 | expressed protein EST gb |
| At1g69630 | F-box protein family contains F-box domain Pfam: PF00646 |
| At1g36950 | zinc finger protein-related similar to GB: AAC69857 from [Arabidopsis thaliana] |
| At1g55550 | kinesin-related protein Similar to Kinesin proteins; Contains kinesin motor |
| | domain protein motif and kinesin heavy chain signature motif |
| At2g23890 | expressed protein and genefinder |
| At2g07030 | Mutator-related transposase similar to MURA transposase of maize Mutator |
| | transposon |
| At2g14810 | hypothetical protein |
| At2g31470 | F-box protein family contains F-box domain Pfam: PF00646 |
| At3g46470 | hypothetical protein |
| At3g06400 | DNA-dependent ATPase, putative similar to DNA-dependent ATPase SNF2H |
| | [Mus musculus] GI: 14028669; contains Pfam profiles PF00271: Helicase |
| | conserved C-terminal domain, PF00176: SNF2 family N-terminal domain, |
| | PF00249: Myb-like DNA-binding domain |
| At3g04430 | No apical meristem (NAM) protein family similar to CUC1 (GP: 12060422) |
| | {Arabidopsis thaliana} |
| At3g51050 | expressed protein hypothetical protein L1648.04 - Leishmania major, |
| | EMBL: LMFL1648 |
| At3g43590 | expressed protein hexamer-binding protein HEXBP - Leishmania major, |
| | PIR: A47156 |
| At3g16360 | two-component phosphorelay mediator-related similar to two-component |
| | phosphorelay mediators (ATHP1-3) GB: BAA37110, GB: BAA37111, |
| | GB: BAA37112 [Arabidopsis thaliana] |
| At4g05260 | ubiquitin family contains INTERPRO: IPR000626 ubiquitin domain |
| At4g32620 | expressed protein predicted protein T10M13.8, Arabidopsis thaliana |
| At5g10110 | expressed protein 85K major surface antigen, Trypanosoma cruzi, PIR: A24154 |
| At5g56340 | expressed protein similar to unknown protein (pir |
| At5g07590 | WD-40 repeat protein family contains 3 WD-40 repeats (PF00400); similarity to |
| | WD-repeat protein 8 (WDR8)(SP: Q9P2S5] [Homo sapiens] |
| At5g50100 | expressed protein similar to unknown protein (pir |
| At5g55690 | MADS-box protein |
| At5g09840 | expressed protein similar to unknown protein (emb |
| At5g42130 | mitochondrial carrier protein family contains Pfam profile: PF00153 |
| | mitochondrial carrier protein |
| At5g22530 | expressed protein |
| At5g40220 | MADS-box protein MADS-box protein, Arabidopsis thaliana, EMBL: ATY12776 |
| At3g44010 | 40S ribosomal protein S29 (RPS29B) ribosomal protein S29, rat, PIR: S30298 |
| At3g45190 | expressed protein hypothetical protein At2g28360 - Arabidopsis thaliana, |
| | EMBL: AAD20690 |
| At5g44410 | FAD-linked oxidoreductase family similar to SP |
| At1g18120 | pseudogene, putative myrosinase-associated protein |
| At1g51860 | leucine rich repeat protein kinase, putative similar to light repressible receptor |
| | protein kinase [Arabidopsis thaliana] gi |
| At1g62690 | expressed protein |
| At5g39000 | protein kinase family contains protein kinase domain, Pfam: PF00069 |
| At4g03970 | Ulp1 protease family contains Pfam profile PF02902: Ulp1 protease family, C- |
| | terminal catalytic domain; similar to At5g28170, At1g35110, At1g44880, |
| | At3g42530, At4g19320, At5g36020, At3g43010, At2g10350 |
| At1g17615 | disease resistance protein (TIR class), putative domain signature TIR exists, |
| | suggestive of a disease resistance protein. |
| At1g56420 | hypothetical protein |
| At1g32870 | NAM protein-related similar to NAM protein GI: 6066594 from [Petunia hybrida] |
| At1g04870 | protein arginine N-methyltransferase family similar to SP |
| At1g61680 | terpene synthase/cyclase family similar to 1,8-cineole synthase |
| | [GI: 3309117][Salvia officinalis]; contains Pfam profile: PF01397 terpene |
| | synthase family |
| At1g25500 | expressed protein |
| At1g68570 | peptide transporter-related similar to PEPTIDE TRANSPORTER PTR2-B |
| | GB: P46032 GI: 1172704 from [Arabidopsis thaliana] |
| At1g06520 | phospholipid/glycerol acyltransferase family contains Pfam profile PF01553: |
| | Acyltransferase |
| At1g54550 | F-box protein family contains Pfam: PF00646 F-box domain; contains |
| | TIGRFAM TIGR01640: F-box protein interaction domain |
| At2g01340 | expressed protein |
| At2g05250 | DnaJ domain-containing protein contains Pfam profile PF00226 DnaJ domain |
| At2g17910 | reverse transcriptase (RNA-dependent DNA polymerase), putative similar to |
| | reverse transcriptase [Arabidopsis thaliana] GI: 976278; contains Pfam profiles |
| | PF00078: Reverse transcriptase (RNA-dependent DNA polymerase), |
| | PF03372: Endonuclease/Exonuclease/p? |
| At2g44130 | Kelch repeat containing F-box protein family very low similarity to SP |
| At2g24670 | hypothetical protein |
| At3g23080 | expressed protein C-term similar to phosphatidylcholine transfer protein |
| | GB: AAF08345 [Homo sapiens] |
| At3g09310 | alpha-hemolysin-related similar to alpha-hemolysin GB: AAB81225 |
| | [Aeromonas hydrophila] |
| At3g53070 | hypothetical protein predicted protein, Arabidopsis thaliana |
| At3g48180 | hypothetical protein |
| At3g28430 | expressed protein GC donor splice site at exon 16 |
| At3g23160 | hypothetical protein |
| At3g23670 | phragmoplast-associated kinesin-related protein, putative similar to kinesin like |
| | protein GB: CAB10194 from [Arabidopsis thaliana] |
| At4g19350 | expressed protein |
| At4g30300 | ABC transporter family protein ribonuclease L inhibitor - Mus |
| | musculus, PIR2: JC6555 |
| At4g00760 | expressed protein |
| At4g28180 | hypothetical protein |
| At4g18320 | hypothetical protein |
| At4g03830 | myosin heavy chain-related |
| At4g18820 | expressed protein DNA polymerase III holoenzyme tau subunit, Thermus |
| | thermophilus, gb: AF025391 |
| At5g12970 | C2 domain-containing protein contains INTERPRO: IPR000008 C2 domain |
| At5g66350 | zinc finger protein SHI-related |
| At5g13080 | WRKY family transcription factor WRKY DNA binding protein - Solanum |
| | tuberosum, EMBL: AJ278507 |
| At5g02460 | Dof zinc finger protein zinc finger protein OBP3, Arabidopsis thaliana, |
| | EMBL: AF155818 |
| At5g22550 | expressed protein strong similarity to unknown protein (emb |
| At5g56910 | expressed protein similar to unknown protein (pir |
| At5g39630 | SNARE protein AtMEMB11 v-SNARE AtVTI1a, Arabidopsis thaliana, |
| | EMBL: AF114750 |
| At3g51090 | expressed protein hypothetical protein F16F14.4 - Arabidopsis thaliana: |
| | EMBL: AC007047 |
| At5g43270 | squamosa promoter binding protein-related 2 (emb |
| At1g54760 | MADS-box protein similar to MADS-box transcription factor GI: 4837612 from |
| | [Antirrhinum majus] |
| At4g01590 | expressed protein |
| At5g15650 | reversibly glycosylated polypeptide-3 |
| At3g19800 | expressed protein |
| At3g26820 | esterase/lipase/thioesterase family contains Interpro entry IPR000379 |
| At5g48400 | glutamate receptor family (GLR1.2) plant glutamate receptor family, |
| | PMID: 11379626 |
| At5g43410 | ethylene response factor, putative contains AP2 DNA-binding domain |
| At2g18860 | expressed protein |
| At1g62200 | oligopeptide transporter-related similar to oligopeptide transporter 1-1 |
| | GI: 510238 from [Arabidopsis thaliana]; contains non-consensus GA donor site |
| | at intron 4 |
| At2g03260 | expressed protein |
| At3g05240 | pentatricopeptide (PPR) repeat-containing protein contains Pfam profile |
| | PF01535: PPR repeat |
| At1g05660 | polygalacturonase, putative similar to GB: AAC23398 |
| At1g48670 | Nt-gh3 deduced protein-related similar to Nt-gh3 deduced protein GI: 4887010 |
| | from [Nicotiana tabacum] |
| At3g14075 | lipase (class 3) family low similarity to calmodulin-binding heat-shock protein |
| | CaMBP [Nicotiana tabacum] GI: 1087073; contains Pfam profile PF01764: |
| | Lipase, PF03893: Lipase 3 N-terminal region |
| At1g31850 | dehydration-induced protein, putative strong similarity to early-responsive to |
| | dehydration stress ERD3 protein [Arabidopsis thaliana] GI: 15320410; contains |
| | Pfam profile PF03141: Putative methyltransferase |
| At1g44760 | expressed protein |
| At1g08500 | plastocyanin-like domain containing protein |
| At1g17650 | expressed protein |
| At1g59640 | bHLH protein |
| At1g72520 | lipoxygenase (LOX), putative similar to lipoxygenase gi: 1495804 [Solanum |
| | tuberosum], gi: 1654140 [Lycopersicon esculentum], GB: CAB56692 |
| | [Arabidopsis thaliana] |
| At1g68500 | expressed protein |
| At1g36490 | pseudogene, putative replication protein A1 |
| At2g27240 | expressed protein contains Pfam profile PF01027: Uncharacterized protein |
| | family UPF0005 |
| At2g07240 | Ulp1 protease family contains Pfam profile PF02902: Ulp1 protease family, C- |
| | terminal catalytic domain |
| At3g14560 | expressed protein |
| At3g22790 | expressed protein similar to centromere protein homolog GB: CAB10255 from |
| | [Arabidopsis thaliana] |
| At3g16010 | pentatricopeptide (PPR) repeat-containing protein contains Pfam profile |
| | PF01535: PPR repeat |
| At3g57540 | expressed protein putative DNA binding protein - Arabidopsis thaliana, |
| | TREMBL: ATAC2339_3 |
| At4g12270 | copper amine oxidase like protein (fragment1) copper amine oxidase - Cicer |
| | arietinum, PID: e1335964 |
| At4g04950 | thioredoxin family similar to PKCq-interacting protein PICOT from [Mus |
| | musculus] GI: 6840949, [Rattus norvegicus] GI: 6840951; contains Pfam profile |
| | PF00085: Thioredoxin |
| At4g20280 | expressed protein transcription initiation factor IID beta chain, fruit fly, |
| | Pir2: B49453 |
| At4g02280 | sucrose synthase (UDP-glucose-fructose glucosyltransferase/sucrose-UDP |
| | glucosyltransferase), putative strong similarity to sucrose synthase |
| | GI: 6682841 from [Citrus unshiu] |
| At5g58787 | C3HC4-type zinc finger protein family similar to MTD2 [Medicago truncatula] |
| | GI: 9294812; contains Pfam profile PF00097: Zinc finger, C3HC4 type (RING |
| | finger) |
| At5g51480 | pectinesterase (pectin methylesterase) family similar to pectinesterase |
| | GB: CAB08077 GI: 1944575 from [Lycopersicon esculentum]; contains Pfam |
| | profile: PF00394 Multicopper oxidase; similar to pollen-specific protein |
| At5g61650 | cyclin family similar to cyclin 2 [Trypanosoma brucei] GI: 7339572, cyclin 6 |
| | [Trypanosoma cruzi] GI: 12005317; contains Pfam profile PF00134: Cyclin, N- |
| | terminal domain |
| At3g53080 | expressed protein BETA-GALACTOSIDASE PRECURSOR. Lycopersicon |
| | esculentum, gb: P48980 |
| At4g35040 | bZIP protein |
| At4g34710 | arginine decarboxylase SPE2 |
| At4g37740 | transcription activator (GRL2) annotation temporarily based on supporting |
| | cDNA gi |
| At1g55210 | disease resistance response protein-related/dirigent protein-related smimilar |
| | to dirigent protein [Thuja plicata] gi |
| At1g49660 | expressed protein |
| At2g34180 | CBL-interacting protein kinase 13 identical to CBL-interacting protein kinase 13 |
| | [Arabidopsis thaliana] gi |
| At1g48070 | hypothetical protein |
| At1g18130 | hypothetical protein contains similarity to threonyl-tRNA synthetases |
| At1g51430 | expressed protein |
| At4g39770 | trehalose-6-phosphate phosphatase, putative similar to trehalose-6-phosphate |
| | phosphatase (AtTPPB) [Arabidopsis thaliana] GI: 2944180; contains Pfam |
| | profile PF02358: Trehalose-phosphatase |
| At4g13760 | polygalacturonase, putative polygalacturonase, Zea mays, PIR2: S30067 |
| At4g00930 | expressed protein |
| At1g61390 | S-locus protein kinase, putative contains protein kinase domain, |
| | Pfam: PF00069; contains S-locus glycoprotein family domain, Pfam: PF00954 |
| At4g34180 | expressed protein hypothetical protein slr2121, Synechocystis sp., |
| | PIR2: S75497 |
| At2g14470 | hypothetical protein low similarity to SP |
| At1g63010 | expressed protein |
| At1g34260 | expressed protein |
| At1g01840 | expressed protein |
| At1g48130 | peroxiredoxin identical to SP: O04005 from [Arabidopsis thaliana] |
| At1g77250 | hypothetical protein |
| At1g52610 | mutator-related transposase similar to mutator-like transposase GI: 5306250 |
| | from [Arabidopsis thaliana] |
| At1g67260 | pseudogene, putative cycloidea cyc4 protein |
| At5g61710 | hypothetical protein predicted protein, Arabidopsis thaliana |
| At1g55110 | zinc finger protein-related similar to zinc finger protein GI: 8843731 from |
| | [Arabidopsis thaliana] |
| At1g05260 | peroxidase, putative similar to peroxidase precursor [Arabidopsis thaliana] gi |
| At1g13290 | zinc finger protein-related similar to zinc finger protein ID1 GI: 3170601 from |
| | [Zea mays] |
| At5g27000 | kinesin-related protein non-consensus AT donor splice site at exon 12; non- |
| | consensus AC acceptor splice site at exon 13 |
| At3g24760 | F-box protein family; similar to SKP1 interacting partner 2 (SKIP2) |
| | TIGR_Ath1: At5g67250 |
| At5g27140 | SAR DNA-binding protein, putative strong similarity to SAR DNA-binding |
| | protein-1 [Pisum sativum] GI: 3132696; contains Pfam profile PF01798: |
| | Putative snoRNA binding domain |
| At2g19570 | cytidine deaminase-related |
| At2g19180 | expressed protein |
| At2g40690 | glycerol-3-phosphate dehydrogenase |
| At2g18640 | geranylgeranyl pyrophosphate synthase |
| | (GGPS2/GGPS5)(farnesyltranstransferase), putative similar to gi: 1944371; |
| | contains GB: L22347 |
| At2g26420 | phosphatidylinositol-4-phosphate 5-kinase-related |
| At2g31140 | expressed protein |
| At2g38840 | guanylate binding protein-related |
| At2g06000 | pentatricopeptide (PPR) repeat-containing protein contains Pfam profile |
| | PEF01535: PPR repeat |
| At3g24780 | hypothetical protein |
| At3g28210 | zinc finger protein (PMZ)-related identical to putative zinc finger protein (PMZ) |
| | GB: AAD37511 GI: 5006473 [Arabidopsis thaliana] |
| At3g23730 | xyloglucan endotransglycosylase, putative similar to xyloglucan |
| | endotransglycosylase-related protein GI: 1244760 from [Arabidopsis thaliana] |
| At3g17880 | thioredoxin, putative similar to SP |
| At3g07940 | hypothetical protein |
| At3g61400 | 2-oxoglutarate-dependent dioxygenase, putative similar to 2A6 (GI: 599622) |
| | and tomato ethylene synthesis regulatory protein E8 (SP |
| At3g44580 | hypothetical protein predicted protein, Arabidopsis thaliana |
| At3g48960 | 60S ribosomal protein L13 (RPL13C) 60S ribosomal protein L13 (BBC1), |
| | Arabidopsis thaliana, gb: X75162 |
| At3g15130 | pentatricopeptide (PPR) repeat-containing protein contains |
| | INTERPRO: IPR002885 PPR repeats |
| At3g24840 | phosphatidylinositol transfer protein-related similar to SEC14 CYTOSOLIC |
| | FACTOR (PHOSPHATIDYLINOSITOL/PHOSPHATIDYLCHOLINE |
| | TRANSFER PROTEIN) GB: P46250 from [Candida albicans] (Yeast (1996) |
| | 12(11), 1097-1105) |
| At3g01710 | hypothetical protein |
| At3g01930 | expressed protein similar to nodule-specific protein NIj70 GB: AAC39500 [Lotus |
| | japonicus] |
| At3g25810 | myrcene/ocimene synthase, putative similar to GI: 9957293; contains Pfam |
| | profile: PF01397 terpene synthase family |
| At3g50460 | hypothetical protein |
| At3g29635 | transferase family similar to anthocyanin 5-aromatic acyltransferase from |
| | Gentiana triflora GI: 4185599, malonyl CoA: anthocyanin 5-O-glucoside-6′″-O- |
| | malonyltransferase from Perilla frutescens GI: 17980232, Salvia splendens |
| | GI: 17980234; contains Pfam pr? |
| At4g31980 | expressed protein EREBP-4 homolog, Arabidopsis thaliana |
| At4g32910 | expressed protein |
| At4g37170 | pentatricopeptide (PPR) repeat-containing protein contains Pfam profile |
| | PF01535: PPR repeat |
| At4g33030 | UDP-sulfoquinovose synthase (sulfite: UDP-glucose sulfotransferase) (sulfolipid |
| | biosynthesis protein) (SQD1) identical to gi: 2736155 |
| At4g04330 | expressed protein |
| At5g24500 | expressed protein |
| At5g48020 | expressed protein |
| At5g54660 | expressed protein |
| At5g44360 | FAD-linked oxidoreductase family similar to SP |
| At5g46160 | ribosomal protein L14p family |
| At5g06540 | pentatricopeptide (PPR) repeat-containing protein contains Pfam profile |
| | PF01535: PPR repeat |
| At5g37200 | C3HC4-type zinc finger protein family low similarity to ring-H2 finger protein |
| | RHY1a from Arabidopsis thaliana [gi: 3790593], ring finger-H2 protein from |
| | Xenopus laevis [gi: 13752371]; contains Pfam domain zinc finger, C3HC4 type |
| | (RING finger) PF00097 |
| At5g63580 | flavonol synthase, putative similar to SP |
| At1g10230 | E3 ubiquitin ligase SCF complex subunit SKP1/ASK1 (At18), putative E3 |
| | ubiquitin ligase; similar to Skp1 homolog Skp1a GI: 3068807 [Arabidopsis |
| | thaliana] |
| At2g35150 | expressed protein |
| At3g10740 | glycosyl hydrolase family 51 similar to arabinoxylan arabinofuranohydrolase |
| | isoenzyme AXAH-II from GI: 13398414 [Hordeum vulgare] |
| At4g26840 | ubiquitin-like protein (SMT3) identical to Ubiquitin-like protein SMT3 |
| | SP: P55852 from[Arabidopsis thaliana] |
| At4g34210 | E3 ubiquitin ligase SCF complex subunit SKP1/ASK1 (At11), putative E3 |
| | ubiquitin ligase; similar to Skp1 homolog Skp1a GI: 3068807 from [Arabidopsis |
| | thaliana] |
| At4g13170 | 60S ribosomal protein L13A (RPL13aC) ribosomal protein L13a - Lupinus |
| | luteus, PID: e1237871 |
| At4g25840 | haloacid dehalogenase-like hydrolase family low similarity to SP |
| At3g27050 | expressed protein |
| At1g60095 | jacalin lectin family contains similarity to myrosinase-binding protein homolog |
| | [Arabidopsis thaliana] GI: 2997767; |
| At4g18480 | magnesium-chelatase, subunit chII, chloroplast (Mg-protoporphyrin IX |
| | chelatase) (CHLI) identical to SP |
| At5g27060 | disease resistance protein family contains leucine rich-repeat (LRR) domains |
| | Pfam: PF00560, INTERPRO: IPR001611; similar to Hcr2-0B [Lycopersicon |
| | esculentum] gi |
| At3g09410 | pectinacetylesterase family similar to pectinacetylesterase precursor |
| | GB: CAA67728 [Vigna radiata]; contains Pfam profile: PF03283 |
| | pectinacetylesterase |
| At1g11655 | expressed protein |
| At1g49800 | hypothetical protein |
| At1g77610 | glucose-6-phosphate/phosphate translocator-related similar to glucose-6- |
| | phosphate/phosphate-translocators from [Mesembryanthemum crystallinum] |
| | GI: 9295277, [Solanum tuberosum] GI: 2997593, [Pisum sativum] GI: 2997591; |
| | contains Pfam profile PF00892: Integ? |
| At1g10380 | expressed protein |
| At1g02860 | expressed protein contains similarity to peroxin-2 GI: 6103008 from [Pichia |
| | pastoris] |
| At1g28410 | expressed protein |
| At1g77350 | expressed protein |
| At1g64480 | calcineurin B-like protein (CBL8) identical to calcineurin B-like protein 8 |
| | (GI: 15866276) [Arabidopsis thaliana]; similar to CALCINEURIN B SUBUNIT |
| | GB: P25296 from [Saccharomyces cerevisiae] |
| At1g16350 | inosine-5′-monophosphate dehydrogenase, putative strong similarity to SP |
| At1g01880 | hypothetical protein contains similarity to DNA repair endonuclease |
| | GB: AAD47568 GI: 5712619 from [Drosophila melanogaster] |
| At1g28100 | expressed protein |
| At1g79400 | cation/proton exchanger, putative (CHX2) monovalent cation: proton antiporter |
| | family 2 (CPA2) member, PMID: 11500563 |
| At1g35650 | UIp1 protease family PF02902: UIp1 protease family, C-terminal catalytic |
| | domain; similar to At1g21020, At3g26530, At1g08760, At1g08740, At2g29240 |
| At1g28560 | expressed protein |
| At2g26870 | phosphoesterase family low similarity to SP |
| At2g13230 | retroelement pol polyprotein-related |
| At2g40580 | protein kinase family contains protein kinase domain, Pfam: PF00069 |
| At2g19700 | hypothetical protein |
| At3g62080 | expressed protein |
| At3g62320 | hypothetical protein hypothetical protein At2g36110 - Arabidopsis thaliana, |
| | EMBL: AC007135 |
| At3g05340 | pentatricopeptide (PPR) repeat-containing protein contains |
| | INTERPRO: IPR002885 PPR repeats |
| At3g47710 | bHLH protein family |
| At3g10160 | dihydrofolate synthetase (dhfs) nearly identical to gi: 17976757 |
| At3g02630 | acyl-[acyl-carrier-protein] desaturase (stearoyl-ACP desaturase), putative |
| | similar to Acyl-[acyl-carrier protein] desaturase from Sesamum indicum |
| | GI: 575942, Cucumis sativus SP |
| At3g08600 | expressed protein |
| At4g03560 | two-pore calcium channel (TPC1) identical to two-pore calcium channel (TPC1) |
| | [Arabidopsis thaliana] gi |
| At4g22640 | expressed protein various predicted proteins, Arabidopsis thaliana |
| At4g01570 | pentatricopeptide (PPR) repeat-containing protein contains Pfam profile |
| | PF01535: PPR repeat |
| At5g13680 | expressed protein similar to unknown protein (ref |
| At5g17420 | cellulose synthase, catalytic subunit (IRX3) identical to gi: 5230423 |
| At5g56850 | expressed protein similar to unknown protein (pir |
| At5g61250 | glycosyl hydrolase family 79 (endo-beta-glucuronidase/heparanase) similar to |
| | beta-glucuronidase GI: 8918740 from [Scutellaria baicalensis] |
| At5g67460 | glycosyl hydrolase family 17 similar to beta-1,3-glucanase GI: 6714534 from |
| | [Salix gilgiana] |
| At5g36200 | hypothetical protein similar to unknown protein (pir |
| At5g54360 | hypothetical protein |
| At5g54150 | hypothetical protein similar to unknown protein (pir |
| At5g46870 | RRM-containing protein similar to unknown protein (pir |
| At5g48700 | ubiquitin family contains INTERPRO: IPR000626 ubiquitin domain |
| At5g23230 | isochorismatase hydrolase family low similarity to SP |
| At5g56040 | leucine rich repeat protein kinase, putative contains leucine rich repeat (LRR) |
| | domains, Pfam: PF00560; contains protein kinase domain, Pfam: PF00069 |
| At5g22870 | hypothetical protein similar to unknown protein (gb |
| At1g73760 | RING zinc finger protein-related contains Pfam profile: PF00097 zinc finger, |
| | C3HC4 type (RING finger) |
| At3g23955 | pseudogene, similar to hypothetical protein GB: AAD29066 |
| At1g31090 | hypothetical protein contains similarity to gi |
| At1g14250 | hypothetical protein |
| At5g39480 | F-box protein family contains Pfam: PF00646 F-box domain similar to SKP1 |
| | interacting partner 2 (SKIP2) TIGR_Ath1: At5g67250 |
| At1g23450 | pentatricopeptide (PPR) repeat-containing protein contains |
| | INTERPRO: IPR002885 PPR repeats |
| At5g51280 | DEAD-box protein abstrakt, putative |
| At1g74190 | leucine rich repeat protein family contains leucine rich-repeat (LRR) domains |
| | Pfam: PF00560, INTERPRO: IPR001611; contains similarity to Cf-2.1 |
| | [Lycopersicon pimpinellifolium] gi |
| At1g71830 | protein kinase-related similar to receptor protein kinase GB: BAA11869 |
| | GI: 1389566 from [Arabidopsis thaliana] |
| At1g51160 | expressed protein |
| At1g17745 | D-3-phosphoglycerate dehydrogenase (3-PGDH) identical to SP |
| At3g26310 | cytochrome P450 family contains Pfam profile: PF00067 cytochrome P450 |
| At1g69910 | protein kinase family contains protein kinase domain, Pfam: PF00069 |
| At1g75850 | vacuolor sorting protein 35-related similar to vacuolar sorting protein 35 |
| | GB: AAF02778 GI: 6049847 [Homo sapiens] |
| At1g18750 | MADS-box protein similar to homeodomain transcription factor (AGL30) |
| | GI: 3461830 from [Arabidopsis thaliana] |
| At5g47620 | heterogeneous nuclear ribonucleoprotein (hnRNP), putative |
| At5g17820 | peroxidase, putative identical to peroxidase ATP13a [Arabidopsis thaliana] gi |
| At5g33370 | GDSL-motif lipase/hydrolase protein similar to family II lipase EXL3 |
| | (GI: 15054386), EXL1 (GI: 15054382), EXL2 (GI: 15054384) [Arabidopsis |
| | thaliana]; contains Pfam profile PF00657: Lipase/Acylhydrolase with GDSL- |
| | like motif |
| At2g39880 | myb family transcription factor (MYB25) contains Pfam profile: PF00249 myb- |
| | like DNA-binding domain |
| At2g20240 | expressed protein |
| At2g02220 | leucine-rich repeat transmembrane protein kinase, putative |
| At2g44210 | expressed protein Pfam profile PF03080: Arabidopsis proteins of unknown |
| | function |
| At3g60150 | hypothetical protein hypothetical protein F4I1.34 - Arabidopsis thaliana, |
| | PIR: T02408 |
| At3g12970 | expressed protein |
| At3g61910 | No apical meristem (NAM) protein family no apical meristem (NAM) - Petunia |
| | hybrida, EMBL: PHDNANAM |
| At3g09030 | expressed protein identical to GB: AAD56319 [Arabidopsis thaliana] |
| At3g02250 | auxin-independent growth promoter-related similar to auxin-independent |
| | growth promoter GB: A44226 [Nicotiana tabacum] |
| At4g10040 | cytochrome c several plant cytochrome c (for instance cucurbit, PIR1: CCPU) |
| At4g23380 | hypothetical protein predicted proteins, Arabidopsis thaliana |
| At4g23110 | hypothetical protein |
| At4g13990 | hypothetical protein |
| At5g54130 | calcium-binding EF-hand family protein contains INTERPRO: IPR002048 |
| | calcium-binding EF-hand domain |
| At5g43670 | protein transport protein SEC23 |
| At5g59800 | hypothetical protein |
| At5g12270 | oxidoreductase, 2OG-Fe(II) oxygenase family similarity to ripening protein E8, |
| | tomato, PIR: S01642; contains Pfam domain PF03171, 2OG-Fe(II) oxygenase |
| | superfamily |
| At5g16530 | auxin efflux carrier protein family contains auxin efflux carrier domain, |
| | Pfam: PF03547 |
| At4g35410 | clathrin assembly protein AP19 homolog |
| At4g03916 | hypothetical protein low similarity to SP |
| At2g14960 | auxin-regulated protein-related |
| At2g15000 | expressed protein |
| At2g16390 | SNF2 domain/helicase domain-containing protein low similarity to RAD54 |
| | [Drosophila melanogaster] GI: 1765914; contains Pfam profiles PF00271: |
| | Helicase conserved C-terminal domain, PF00176: SNF2 family N-terminal |
| | domain |
| At1g51150 | DegP protease contains similarity to DegP2 protease GI: 13172275 from |
| | [Arabidopsis thaliana] |
| At1g66180 | expressed protein |
| At3g60830 | actin - like protein actin 3, Drosophila melanogaster, PIR: A03000 |
| At2g21770 | cellulose synthase, catalytic subunit, putative similar to gi: 2827141 cellulose |
| | synthase catalytic subunit, Arabidopsis thaliana (Ath-A) |
| AtCg00700 | psbN: photosystem II protein N |
| At1g09240 | nicotianamine synthase, putative similar to nicotianamine synthase |
| | [Lycopersicon esculentum][GI: 4753801], nicotianamine synthase 2 [Hordeum |
| | vulgare][GI: 4894912] |
| At1g55120 | glycosyl hydrolase family 32 identical to beta-fructofuranosidase GI: 6683112 |
| | from [Arabidopsis thaliana] |
| At1g77100 | peroxidase, putative similar to cationic peroxidase [Arachis hypogaea] gi |
| At1g68380 | expressed protein |
| At1g53625 | expressed protein |
| At1g27060 | hypothetical protein contains Pfam profile: PF00415 Regulator of chromosome |
| | condensation (RCC1) (7 copies) |
| At1g59530 | bZIP protein similar to G-box binding factor 1 GI: 16286 from (Arabidopsis |
| | thaliana) |
| At5g26780 | glycine hydroxymethyltransferase - like protein glycine |
| | hydroxymethyltransferase, Solanum tuberosum, EMBL: Z25863 |
| At1g22910 | RNA recognition motif (RRM) - containing protein contains InterPro entry |
| | IPR000504: RNA-binding region RNP-1 (RNA recognition motif) (RRM); |
| | similar to GB: AAC33496 |
| At1g13500 | hypothetical protein |
| At2g35980 | harpin-induced protein 1 family (HIN1) similar to harpin-induced protein hin1 |
| | (GI: 1619321) [Nicotiana tabacum] |
| At3g17200 | hypothetical protein similar to potential non-LTR retroelement reverse |
| | transcriptases |
| At1g42460 | Ulp1 protease family contains Pfam profile PF02902: Ulp1 protease family, C- |
| | terminal catalytic domain |
| At3g24830 | 60S ribosomal protein L13A (RPL13aB) similar to 60S RIBOSOMAL PROTEIN |
| | L13A GB: P35427 from [Rattus norvegicus] |
| At3g15590 | DNA-binding protein, putative similar to DNA-binding protein [Triticum |
| | aestivum] GI: 6958202; contains Pfam profile: PF01535 PPR repeat |
| At3g45090 | expressed protein 2-phosphoglycerate kinase - Methanococcus jannaschii, |
| | PIR: A64485 |
| At4g35510 | expressed protein |
| At3g09670 | PWWP domain protein |
| At3g20730 | pentatricopeptide (PPR) repeat-containing protein contains |
| | INTERPRO: IPR002885 PPR repeats |
| At3g44200 | protein kinase family contains protein kinase domain, Pfam: PF00069; contains |
| | serine/threonine protein kinase domain, INTERPRO: IPR002290 |
| At3g11310 | hypothetical protein |
| At3g59180 | hypothetical protein |
| At4g09350 | DnaJ protein family similar to SP |
| At4g36840 | Kelch repeat-containing protein contains Pfam profile PF01344: Kelch motif |
| At4g12850 | hypothetical protein stong similarity only to other predicted proteins from |
| | Arabidopsis and tomato |
| At5g39880 | expressed protein |
| At5g45070 | disease resistance protein (TIR class), putative domain signature TIR exists, |
| | suggestive of a disease resistance protein. |
| At5g49480 | sodium-inducible calcium-binding protein identical to NaCl-inducible Ca2+- |
| | binding protein GI: 2352828 from [Arabidopsis thaliana] |
| At5g53290 | AP2 domain transcription factor, putative contains similarity to pathogenesis- |
| | related genes transcriptional activator |
| At5g67490 | expressed protein |
| At5g58860 | cytochrome P450 86A1 identical to Cytochrome P450 86A1 (CYPLXXXVI) |
| | (P450-dependent fatty acid omega-hydroxylase) (SP: P48422) [Arabidopsis |
| | thaliana] |
| At5g24680 | expressed protein similar to unknown protein (pir |
| At5g16070 | chaperonin, putative similar to SWISS-PROT: P80317 T-complex protein 1, |
| | zeta subunit (TCP-1-zeta) [Mus musculus]; contains Pfam: PF00118 domain, |
| | TCP-1/cpn60 chaperonin family |
| At5g22060 | DnaJ protein, putative strong similarity to SP |
| At5g48710 | ubiquitin family contains INTERPRO: IPR000626 ubiquitin domain |
| At5g47660 | expressed protein contains similarity to DNA-binding protein GT |
| At1g35310 | Bet v I allergen family similar to Csf-2 [Cucumis sativus][GI: 5762258][J Am Soc |
| | Hortic Sci 124, 136-139 (1999)]; contains Pfam profile PF00407: |
| | Pathogenesis-related protein Bet v I family |
| At3g52680 | F-box protein family contains F-box domain Pfam: PF00646 |
| At4g16140 | proline-rich protein family contains proline-rich extensin domains, |
| | INTERPRO: IPR002965 |
| At1g51175 | pseudogene, similar to polyprotein (gypsy_Ty3-element) [Sorghum bicolor] |
| | (GB: AAD19359) |
| At2g35170 | expressed protein |
| At1g52580 | membrane protein, Rhomboid family contains PFAM domain PF01694, |
| | Rhomboid family |
| At2g23300 | leucine-rich repeat transmembrane protein kinase, putative |
| At4g03680 | hypothetical protein |
| At5g36070 | hypothetical protein strong similarity to unknown protein (emb |
| At5g49780 | leucine-rich repeat transmembrane protein kinase, putative |
| At2g36010 | E2FA transcription factor |
| At1g57800 | expressed protein similar to putative zinc finger protein GI: 7267501 from |
| | [Arabidopsis thaliana] |
| At1g37150 | biotin holocarboxylase synthetase-related similar to biotin holocarboxylase |
| | synthetase GI: 4874309 from [Arabidopsis thaliana] contains non-consensus |
| | GG acceptor splice sites. |
| At1g79710 | hypothetical protein similar to hypothetical protein GB: AAC12874 |
| | [Synechococcus PCC7942] |
| At1g73340 | cytochrome P450 family similar to Cytochrome P450 90A1 (SP: Q42569) |
| | [Arabidopsis thaliana]; contains Pfam profile: PF00067 cytochrome P450 |
| At1g26260 | bHLH protein similar to bHLH transcription factor GBOF-1 GI: 5923912 from |
| | [Tulipa gesneriana] |
| At1g44160 | DnaJ protein family contains Pfam profile PF01556: DnaJ C terminal region |
| At1g16420 | hypothetical protein common family similar to At5g04200, At1g79340, |
| | At1g79320, At1g79310, At1g79330; similar to latex-abundant protein |
| | [GI: 4235430][Hevea brasiliensis] |
| At1g71810 | expressed protein |
| At5g47635 | expressed protein |
| At2g33310 | auxin-responsive protein IAA13 (Indoleacetic acid-induced protein 13) identical |
| | to SP |
| At2g22100 | RRM-containing RNA-binding protein |
| At2g15490 | glucosyltransferase-related |
| At2g04380 | hypothetical protein |
| At2g01430 | homeodomain-leucine zipper protein ATHB-17 (HD-Zip transcription factor |
| | Athb-17) identical to (GI: 18857716) homeodomain-leucine zipper protein |
| | ATHB-17 (GI: 18857716) [Arabidopsis thaliana] |
| At3g45680 | transporter protein-related peptide transport protein - Hordeum vulgare, |
| | PIR: T04378 |
| At3g62680 | proline-rich protein family contains proline-rich region, INTERPRO: IPR000694 |
| At3g57430 | pentatricopeptide (PPR) repeat-containing protein contains Pfam profile |
| | PF01535: PPR repeat |
| At3g22180 | DHHC-type zinc finger domain-containing protein contains Pfam profile |
| | PF01529: DHHC zinc finger domain |
| At3g21465 | adenyl cyclase-related similar to adenyl cyclase GB: AAB87670 from [Nicotiana |
| | tabacum] |
| At3g23630 | expressed protein contains Pfam profile: PF01715 IPP transferase |
| At3g19770 | expressed protein |
| At3g55580 | regulator of chromosome condensation-related protein UVB-resistance protein |
| | UVR8, Arabidopsis thaliana, EMBL: AF130441 |
| At4g30900 | expressed protein |
| At4g16420 | transcriptional adaptor like protein |
| At1g19490 | bZIP protein |
| At1g47705 | pseudogene, putative peroxidase similar to peroxidase GB: P00434 GI: 464365 |
| | from [Brassica rapa] |
| At1g10870 | ARF GTPase-activating domain-containing protein |
| At1g07250 | glycosyltransferase family similar to UDP-glucose glucosyltransferase |
| | GI: 453245 from [Manihot esculenta] |
| At1g64105 | No apical meristem (NAM) protein family contains Pfam PF02365: No apical |
| | meristem (NAM) domain |
| At1g58450 | FKBP-type peptidylprolyl isomerase family similar to rof1 from (Arabidopsis |
| | thaliana) GI: 1373396, GI: 1354207; contains Pfam profile PF00515 TPR |
| | Domain |
| At5g33402 | retroelement pol polyprotein-related temporary automated functional |
| | assignment |
| At1g60800 | receptor-related kinase similar to somatic embryogenesis receptor-like kinase |
| | GI: 2224910 from [Daucus carota] |
| At2g32140 | disease resistance protein (TIR class), putative domain signature TIR exists, |
| | suggestive of a disease resistance protein. |
| At2g33090 | hypothetical protein |
| At2g47280 | pectinesterase family contains Pfam profile: PF01095 pectinesterase |
| At2g41020 | expressed protein |
| At2g34800 | hypothetical protein |
| At2g44310 | calcium-binding EF-hand family protein contains INTERPRO: IPR002048 |
| | calcium-binding EF-hand domain |
| At2g41450 | GCN5-related N-acetyltransferase (GNAT) family low similarity to Swift |
| | [Xenopus laevis] GI: 14164561; contains Pfam profiles PF00583: |
| | acetyltransferase, GNAT family, PF00533: BRCA1 C Terminus (BRCT) |
| | domain |
| At3g23175 | expressed protein supported by RACE-based full-length cDNA validates this |
| | gene structure. (Brassica genome sequence alignment supported. Work by |
| | cdtown, et al.) |
| At3g42780 | hypothetical protein hypothetical protein MZB10.16 - Arabidopsis thaliana, |
| | EMBL: AC009326 |
| At3g20840 | ovule development protein, putative similar to ovule development protein |
| | AINTEGUMENTA (GI: 1209099)[Arabidopsis thaliana] |
| At4g36750 | quinone reductase family similar to 1,4-benzoquinone reductase |
| | [Phanerochaete chrysosporium][GI: 4454993]; similar to Trp repressor binding |
| | protein [Escherichia coli][SP |
| At4g29000 | transcription factor-related leghemoglobin activating factor - Glycine max, |
| | PID: e1374538 |
| At5g22420 | acyl CoA reductase-related protein |
| At5g60610 | F-box protein family contains F-box domain Pfam: PF00646 |
| At5g01370 | hypothetical protein |
| At5g03960 | calmodulin-binding protein-related |
| At5g52050 | MATE efflux protein-related contains Pfam profile PF01554: Uncharacterized |
| | membrane protein family |
| At5g03160 | expressed protein P58 protein, Bos primigenius taurus, PIR: A56534 |
| At5g66970 | hypothetical protein contains similarity to signal recognition particle 54 K |
| | protein |
| At5g65290 | expressed protein |
| At5g60670 | 60S ribosomal protein L12 (RPL12C) 60S RIBOSOMAL PROTEIN L12 (like), |
| | Arabidopsis thaliana, PIR: T45883 |
| At2g40150 | expressed protein |
| At4g29430 | 40S ribosomal protein S15A (RPS15aE) ribosomal protein S15a - Brassica |
| | napus, PIR2: S20945 |
| At2g12700 | hypothetical protein similar to GB: AAD23022 |
| At4g05520 | calcium-binding EF-hand family protein similar to EH-domain containing |
| | protein 1 from {Mus musculus} SP |
| At3g24020 | disease resistance response protein-related contains similarity to disease |
| | resistance response protein 206-d [Pisum sativum] gi |
| At5g63690 | hypothetical protein |
| At2g29290 | short-chain dehydrogenase/reductase family protein (tropinone reductase, |
| | putative) similar to tropinone reductase SP: P50165 from [Datura stramonium] |
| At2g25130 | expressed protein contains Pfam profile: PF00514 Armadillo/beta-catenin-like |
| | repeat |
| At1g67850 | F12A21.2 hypothetical protein |
| At3g46670 | glucosyltransferase-related protein UDP-glucose glucosyltransferase - |
| | Arabidopsis thaliana, EMBL: AB016819 |
| At4g18010 | inositol polyphosphate 5-phosphatase II (IP5PII) nearly identical to inositol |
| | polyphosphate 5-phosphatase II [Arabidopsis thaliana] GI: 10444263 isoform |
| | contains an AT-acceptor splice site at intron 6 |
| At1g80480 | expressed protein contains Viral RNA helicase domain |

TABLE II

| TAIR | |
|---|---|
| accession | |
| No. | Description (homologous genes identified in other organisms) |
| At3g54400 | nucleoid DNA-binding - like protein nucleoid DNA-binding protein cnd41, |
| | chloroplast, common tobacco, PIR: T01996 |
| At2g15570 | thioredoxin M-type 3, chloroplast precursor (TRX-M3) identical to SP |
| | trnY&trnE |
| At5g66140 | 20S proteasome alpha subunit D2 (PAD2) (gb |
| At2g40510 | 40S ribosomal protein S26 (RPS26A) |
| At1g80300 | adenine nucleotide translocase identical to adenine nucleotide translocase |
| | GB: Z49227 [Arabidopsis thaliana] (FEBS Lett. 374 (3), 351-355 (1995)) |
| At1g17260 | ATPase 10, plasma membrane-type (proton pump 10) (proton-exporting |
| | ATPase), putative strong similarity to SP |
| At4g15440 | hydroperoxide lyase (HPOL) like protein |
| At4g22260 | alternative oxidase, putative (IMMUTANS) identical to IMMUTANS from |
| | Arabidopsis thaliana [gi: 4138855]; contains Pfam profile PF01786 alternative |
| | oxidase |
| At3g27690 | light harvesting chlorophyll A/B binding protein, putative similar to chlorophyll A- |
| | B binding protein 151 precursor (LHCP) GB: P27518 from [Gossypium |
| | hirsutum] |
| At3g56690 | calmodulin-binding protein identical to calmodulin-binding protein GI: 6760428 |
| | from [Arabidopsis thaliana] |
| At3g15640 | cytochrome c oxidase subunit Vb-related similar to cytochrome oxidase IV |
| | GB: 223590 [Bos taurus]; contains Pfam profile: PF01215 cytochrome c oxidase |
| | subunit Vb |
| At3g48425 | endonuclease/exonuclease/phosphatase family similar to SP |
| At2g31670 | expressed protein |
| At4g26670 | expressed protein |
| At1g06380 | expressed protein similar to hypothetical protein GI: 6598642 from [Arabidopsis |
| | thaliana] |
| At4g11960 | hypothetical protein hypothetical protein F7H19.70 - Arabidopsis thaliana, |
| | PID: e1310057 |
| At1g78620 | expressed protein |
| At3g48730 | glutamate-1-semialdehyde 2,1-aminomutase 2 (GSA 2) (glutamate-1- |
| | semialdehyde aminotransferase 2) (GSA-AT 2) identical to GSA2 [SP |
| At5g63570 | glutamate-1-semialdehyde 2,1-aminomutase 1 (GSA 1) (glutamate-1- |
| | semialdehyde aminotransferase 1) (GSA-AT 1) identical to GSA 1 [SP |
| At3g44780 | hypothetical protein |
| At4g28660 | photosystem II protein W - like photosystem II protein W, Porphyra purpurea, |
| | PIR2: S73268 |
| At5g44020 | vegetative storage protein-related |
| At1g12170 | F-box protein family contains F-box domain Pfam: PF00646 |
| At1g46768 | AP2 domain protein RAP2.1 identical to AP2 domain containing protein RAP2.1 |
| | GI: 2281627 from [Arabidopsis thaliana] |
| At1g13620 | hypothetical protein |
| At1g77720 | protein kinase family contains protein kinase domain, Pfam: PF00069 |
| At1g35530 | DEAD/DEAH box helicase, putative low similarity to RNA helicase/RNAseIII CAF |
| | protein [Arabidopsis thaliana] GI: 6102610; contains Pfam profiles PF00270: |
| | DEAD/DEAH box helicase, PF00271: Helicase conserved C-terminal domain |
| At1g55570 | pectinesterase (pectin methylesterase) family similar to pectinesterase |
| | [Lycopersicon esculentum][GI: 1944575]; nearly identical to pollen-specific |
| | BP10 protein [SP |
| At1g14000 | protein kinase-related |
| At1g35500 | hypothetical protein |
| At1g21170 | hypothetical protein |
| At1g72330 | alanine aminotransferase, putative similar to alanine aminotransferase 2 SP |
| At1g18040 | cell division protein kinase, putative similar to cell division protein kinase 7 |
| | [Homo sapiens] SWISS-PROT: P50613 |
| At1g08340 | rac GTPase activating protein-related similar to rac GTPase activating protein 1 |
| | GI: 3695059 from [Lotus japonicus] |
| At1g27260 | hypothetical protein |
| At4g38620 | transcription factor (MYB4)-related |
| At2g47460 | myb family transcription factor similar to myb-related DNA-binding protein |
| | GI: 1020155 from [Arabidopsis thaliana] |
| At2g18010 | auxin-induced (indole-3-acetic acid induced) protein family similar to auxin- |
| | induced protein TGSAUR22 (GI: 10185820) [Tulipa gesnerian]; similar to indole- |
| | 3-acetic acid induced protein ARG7 (SP: P32295) [Phaseolus aureus] |
| At2g36840 | ACT domain-containing protein contains Pfam profile ACT domain PF01842 |
| At2g37080 | myosin heavy chain-related |
| At2g31280 | expressed protein |
| At3g57380 | expressed protein hypothetical protein T32G6.16 - Arabidopsis thaliana, |
| | PIR: T00820 |
| At3g57250 | hypothetical protein |
| At3g51470 | protein phosphatase 2C (PP2C), putative protein phosphatase-2C, |
| | Mesembryanthemum crystallinum, EMBL: AF075580 |
| At3g45990 | actin depolymerising like protein Actin depolymerising factor 2, Arabidopsis |
| | thaliana, EMBL: ATU48939 |
| At3g47970 | hypothetical protein |
| At4g23780 | hypothetical protein Arabidopsis hypothetical proteins |
| At3g20350 | expressed protein |
| At4g27620 | expressed protein |
| At4g29700 | nucleotide pyrophosphatase-related protein nucleotide pyrophosphatase, Oryza |
| | sativa, gb: T03293 |
| At4g36900 | AP2 domain protein RAP2.10 Identical to GP: 2632063 and GP: 7270639 |
| | [Arabidopsis thaliana] |
| At4g02150 | importin alpha-2 subunit identical to importin alpha-2 subunit (Karyopherin alpha- |
| | 2 subunit) (KAP alpha) SP: O04294 from [Arabidopsis thaliana] |
| At5g03310 | auxin-induced (indole-3-acetic acid induced) protein family similar to indole-3- |
| | acetic acid induced protein ARG7 (SP: P32295) [Vigna radiata] |
| At5g16730 | expressed protein predicted proteins - Arabidopsis thaliana and Oryza sativa |
| At5g37450 | leucine-rich repeat transmembrane protein kinase, putative |
| At5g47520 | GTP-binding protein, putative similar to GTP-binding protein RAB11J |
| | GI: 1370160 from [Lotus japonicus] |
| At5g51360 | hypothetical protein |
| At2g37640 | expansin, putative (EXP3) identical to Alpha-expansin 3 precursor (At- |
| | EXP3)[Arabidopsis thaliana] SWISS-PROT: O80932; alpha-expansin gene |
| | family, PMID: 11641069 |
| At2g18040 | peptidyl-prolyl cis-trans isomerase-related similar to ESS1 (S. cerevisiae) and |
| | dodo (D. melanogaster.) |
| At4g39690 | expressed protein |
| At5g38480 | 14-3-3 protein GF14 psi (grf3/RCI1) identical to 14-3-3 protein GF14 psi |
| | GI: 1168200, SP: P42644 |
| At2g11930 | pseudogene, hypothetical protein and genefinder |
| At1g53730 | leucine-rich repeat transmembrane protein kinase 1, putative similar to |
| | GI: 3360289 from [Zea mays] (Plant Mol. Biol. 37 (5), 749-761 (1998)) |
| At1g61580 | 60S ribosomal protein L3 (RPL3B) identical to ribosomal protein GI: 806279 from |
| | [Arabidopsis thaliana] |
| At1g30450 | cation-chloride cotransporter, putative similar to cation-chloride co-transporter |
| | GB: AAC49874 GI: 2582381 from [Nicotiana tabacum], Cation-Chloride |
| | Cotransporter (CCC) Family Member, PMID: 11500563 |
| At1g76110 | expressed protein |
| At1g17880 | transcription factor-related similar to transcription factor BTF3 homolog |
| | GI: 2982299 from [Picea mariana] |
| At1g04880 | expressed protein |
| At1g54490 | exonuclease-related similar to 5′-3′ exonuclease GI: 1894792 from [Mus |
| | musculus] |
| At2g31320 | poly (ADP-ribose) polymerase-related |
| At2g38500 | expressed protein |
| At2g02180 | TOM3 protein annotation temporarily based on supporting cDNA gi |
| At2g16860 | expressed protein |
| At4g31980 | expressed protein EREBP-4 homolog, Arabidopsis thaliana |
| At3g15700 | hypothetical protein similar to N-term of NBS/LRR disease resistance protein |
| | GB: AAC26125 [Arabidopsis thaliana]; contains Pfam profile: PF00931 NB-ARC |
| | domain |
| At3g21933 | pseudogene contains Pfam profile: PF01657 Domain of unknown function |
| At3g17470 | calcium-binding EF-hand family protein contains INTERPRO: IPR002048 |
| | calcium-binding EF-hand domain |
| At3g60520 | expressed protein |
| At3g08560 | vacuolar ATP synthase subunit E-related similar to vacuolar ATP synthase |
| | subunit E GB: Q39258 [Arabidopsis thaliana] |
| At3g53330 | plastocyanin-like domain containing protein similar to mavicyanin SP: P80728 |
| | from [Cucurbita pepo] |
| At4g15040 | subtilisin-like serine protease contains similarity to prepro-cucumisin GI: 807698 |
| | from [Cucumis melo] |
| At4g10740 | hypothetical protein |
| At4g37130 | proline-rich protein-related |
| At5g37690 | lipase family similar to family II lipase EXL3 (GI: 15054386), EXL1 |
| | (GI: 15054382), EXL2 (GI: 15054384) [Arabidopsis thaliana] |
| At5g46000 | jacalin lectin family similar to myrosinase-binding protein homolog [Arabidopsis |
| | thaliana] GI: 2997767; contains Pfam profile PF01419 jacalin-like lectin domain |
| At5g54310 | ARF GAP-like zinc finger-containing protein (ZIGA3) almost identical to ARF |
| | GAP-like zinc finger-containing protein ZIGA3 GI: 10441352 from [Arabidopsis |
| | thaliana] |
| At5g15490 | UDP-glucose dehydrogenase-related protein UDP-glucose 6-dehydrogenase - |
| | Glycine max, EMBL: U53418 |
| At4g13510 | ammonium transport protein (AMT1) |
| At4g02630 | protein kinase family contains protein kinase domain, Pfam: PF00069; contains |
| | serine/threonine protein kinase domain, INTERPRO: IPR002290 |
| At1g56100 | hypothetical protein |
| At1g74150 | Kelch repeat-containing protein low similarity to rngB protein, Dictyostelium |
| | discoideum, PIR: S68824; contains Pfam profile PF01344: Kelch motif |
| At1g69770 | chromomethylase-related similar to chromomethylase GB: AAB95486 |
| | [Arabidopsis arenosa] |
| At3g30810 | hypothetical protein |
| At5g18620 | DNA-dependent ATPase, putative similar to DNA-dependent ATPase SNF2H |
| | [Mus musculus] GI: 14028669; contains Pfam profiles PF00271: Helicase |
| | conserved C-terminal domain, PF00176: SNF2 family N-terminal domain, |
| | PF00249: Myb-like DNA-binding domain |
| At1g62050 | expressed protein |
| At3g25940 | expressed protein |
| At1g80050 | adenine phosphoribosyltransferase almost identical to adenine |
| | phosphoribosyltransferase GI: 1402894 from [Arabidopsis thaliana] |
| At1g59312 | hypothetical protein |
| At1g64960 | expressed protein |
| At1g03370 | C2 domain/GRAM domain-containing protein low similarity to SP |
| At1g03590 | protein phosphatase 2C (PP2C) similar to GB: AAB97706 |
| At4g17910 | hypothetical protein predicted protein, Saccharomyces cerevisiae, PIR2: S56868 |
| At2g33580 | protein kinase-related contains a protein kinase domain profile (PDOC00100) |
| At2g44190 | expressed protein |
| At2g18480 | mannitol transporter, putative similar to mannitol transporter [Apium graveolens |
| | var. dulce] GI: 12004316; contains Pfam profile PF00083: major facilitator |
| | superfamily protein |
| At2g46310 | AP2 domain transcription factor, putative |
| At3g09600 | myb family transcription factor contains Pfam profile: PF00249 myb-like DNA- |
| | binding domain |
| At3g26090 | expressed protein |
| At3g13224 | RNA recognition motif (RRM) - containing protein contains InterPro entry |
| | IPR000504: RNA-binding region RNP-1 (RNA recognition motif) (RRM) |
| At3g54220 | scarecrow transcription factor (SCR) |
| At3g61510 | 1-aminocyclopropane-1-carboxylate synthase (ACC synthase), putative similar |
| | to ACC synthases from Citrus sinensis [GI: 6434142], Cucumis melo |
| | [GI: 695402], Cucumis sativus [GI: 3641645] |
| At3g46020 | RNA-binding protein, putative similar to Cold-inducible RNA-binding protein |
| | (Glycine-rich RNA-binding protein CIRP) from {Homo sapiens} SP |
| At4g28780 | GDSL-motif lipase/hydrolase protein similar to family II lipase EXL3 |
| | (GI: 15054386), EXL1 (GI: 15054382), EXL2 (GI: 15054384) [Arabidopsis |
| | thaliana]; contains Pfam profile PF00657: Lipase/Acylhydrolase with GDSL-like |
| | motif |
| At4g13650 | pentatricopeptide (PPR) repeat-containing protein contains Pfam profile |
| | PF01535: PPR repeat |
| At4g09580 | expressed protein hypothetical protein - Arabidopsis thaliana, PIR2: B71448 |
| At5g04220 | C2 domain-containing protein GC donor splice site at exon 3; similar to Ca2+- |
| | dependent lipid-binding protein (CLB1) GI: 2789434 from [Lycopersicon |
| | esculentum] |
| At5g18240 | transfactor-related protein |
| At4g10020 | short-chain dehydrogenase/reductase family protein similar to sterol-binding |
| | dehydrogenase steroleosin GI: 15824408 from [Sesamum indicum] |
| At5g20730 | auxin response transcription factor (ARF7) identical to auxin response factor 7 |
| | GI: 4104929 from [Arabidopsis thaliana] |
| At5g65630 | bromodomain-containing protein similar to 5.9 kb fsh membrane protein |
| | [Drosophila melanogaster] GI: 157455; contains Pfam profile PF00439: |
| | Bromodomain |
| At1g78300 | 14-3-3 protein GF14 omega (grf2) identical to GF14omega isoform GI: 487791 |
| | from [Arabidopsis thaliana] |
| At1g61960 | expressed protein similar to hypothetical protein GI: 5541664 from [Arabidopsis |
| | thaliana] |
| At2g14630 | hypothetical protein contains Pfam profile PF03004: Plant transposase |
| | (Ptta/En/Spm family) |
| At5g16230 | acyl-[acyl-carrier-protein] desaturase (stearoyl-ACP desaturase), putative similar |
| | to Acyl-[acyl-carrier protein] desaturase from Spinacia oleracea SP |
| At1g22170 | expressed protein contains similarity to phosphoglycerate mutases |
| At4g08320 | tetratricopeptide repeat (TPR)-containing protein glutamine-rich tetratricopeptide |
| | repeat (TPR) containing protein (SGT) - Rattus norvegicus, PID: e1285298 (SP |
| At5g49500 | SRP54 (signal recognition particle 54 KDa) protein |
| At3g49400 | transducin/WD-40 repeat protein family contains 4 WD-40 repeats (PF00400); |
| | low similarity (47%) to Agamous-like MADS box protein AGL5 (SP: P29385) |
| | {Arabidopsis thaliana} |
| At1g22210 | trehalose-6-phosphate phosphatase, putative similar to trehalose-6-phosphate |
| | phosphatase (AtTPPB) GI: 2944180 from [Arabidopsis thaliana]; contains Pfam |
| | profile PF02358: Trehalose-phosphatase |
| At1g68935 | expressed protein |
| At1g24625 | zinc finger protein 7, ZFP7 |
| At1g08100 | high-affinity nitrate transporter ACH2 identical to GB: AAC35884 from |
| | [Arabidopsis thaliana] (Plant J. 17 (5), 563-568 (1999)) |
| At1g71750 | hypoxanthine ribosyl transferase-related similar to hypoxanthine ribosyl |
| | transferase GB: AAC46403 GI: 2689037 from [Vibrio parahaemolyticus] |
| At4g38240 | alpha-1,3-mannosyl-glycoprotein beta-1,2-N-acetylglucosaminyltransferase, |
| | putative similar to N-acetylglucosaminyltransferase I from Arabidopsis thaliana |
| | [gi: 5139335]; contains AT-AC non-consensus splice sites at intron 13 |
| At5g59613 | expressed protein |
| At2g19000 | expressed protein |
| At3g02810 | protein kinase family contains protein kinase domain, Pfam: PF00069 |
| At3g09080 | transducin/WD-40 repeat protein family contains 8 WD-40 repeats; similar to |
| | JNK-binding protein JNKBP1 (GP: 6069583) [Mus musculus] |
| At3g06160 | transcriptional factor B3 family contains Pfam profile PF02362: B3 DNA binding |
| | domain |
| At3g61450 | syntaxin of plants 73 (SYP73) annotation temporarily based on supporting cDNA |
| | gi |
| At3g12540 | hypothetical protein |
| At3g26800 | hypothetical protein |
| At3g15510 | No apical meristem (NAM) protein family contains Pfam PF02365: No apical |
| | meristem (NAM) domain; similar to jasmonic acid 2 GB: AAF04915 from |
| | [Lycopersicon esculentum] |
| At3g56790 | hypothetical protein hypothetical protein F27K19.110 - Arabidopsis thaliana, |
| | PIR: T49205 |
| At4g15890 | expressed protein |
| At4g09510 | neutral invertase like protein Daucus carota mRNA, PID: e1372926 |
| At5g58000 | expressed protein similar to unknown protein (gb |
| At5g39790 | expressed protein 5′-AMP-ACTIVATED PROTEIN KINASE, BETA-1 SUBUNIT, |
| | pig, SWISSPROT: AAKB_PIG |
| At5g53210 | bHLH protein family contains similarity to helix-loop-helix DNA-binding protein |
| At5g51030 | short-chain dehydrogenase/reductase family protein contains INTERPRO family |
| | IPR002198 short chain dehydrogenase/reductase SDR family |
| At5g05190 | expressed protein similar to unknown protein (emb |
| At3g12600 | MutT/nudix family protein contains Pfam profile PF00293: NUDIX domain |
| At3g54180 | cell division control protein 2 homolog B (CDC2B) identical to cell division control |
| | protein 2 homolog B [Arabidopsis thaliana] SWISS-PROT: P25859 |
| At2g33530 | serine carboxypeptidase-related |
| At3g09110 | hypothetical protein |
| At4g27130 | translation initiation factor |
| At1g60220 | UIp1 protease family contains Pfam profile PF02902: UIp1 protease family, C- |
| | terminal catalytic domain |
| At1g49140 | NADH-ubiquinone oxidoreductase 12 kD subunit-related annotation temporarily |
| | based on supporting cDNA gi |
| At1g52700 | hypothetical protein contains similarity to lysophospholipase GI: 1552244 from |
| | [Rattus norvegicus] |
| At4g39430 | hypothetical protein |
| At4g35600 | protein kinase family contains protein kinase domain, Pfam: PF00069 |
| At2g18980 | peroxidase, putative identical to peroxidase ATP22a [Arabidopsis thaliana] gi |
| At2g27410 | hypothetical protein |
| At2g14520 | CBS domain containing protein contains Pfam profiles PF00571: CBS domain, |
| | PF01595: Domain of unknown function |
| At2g19190 | light repressible receptor protein kinase, putative similar to light repressible |
| | receptor protein kinase [Arabidopsis thaliana] gi |
| At2g18070 | hypothetical protein |
| At2g41970 | protein kinase, putative similar to Pto kinase interactor 1 (serine/threonine |
| | protein kinase) [Lycopersicon esculentum] gi |
| At3g28030 | UV hypersensitive protein (UVH3) annotation temporarily based on supporting |
| | cDNA gi |
| At3g56490 | protein kinase C inhibitor-related protein protein kinase C inhibitor - Zea mays, |
| | PIR: S45368 |
| At3g29280 | hypothetical protein |
| At3g15310 | expressed protein |
| At3g29570 | hypothetical protein |
| At4g00770 | expressed protein |
| At4g38270 | glycosyltransferase family 8 contains Pfam profile: PF01501 glycosyl transferase |
| | family 8 |
| At4g11930 | hypothetical protein |
| At4g36560 | hypothetical protein |
| At4g08470 | mitogen-activated protein kinase, putative similar to mitogen-activated protein |
| | kinase [Arabidopsis thaliana] gi |
| At4g40000 | proliferating-cell nucleolar antigen - like protein proliferating-cell nucleolar |
| | antigen, Saccharomyce scerevisiae, PIR2: S45758 |
| At4g04180 | vesicle transfer ATPase-related |
| At5g53710 | expressed protein |
| At5g03890 | hypothetical protein predicted protein, Arabidopsis thaliana |
| At5g22510 | alkaline/neutral invertase |
| At5g48660 | hypothetical protein contains similarity to unknown protein (gb |
| At5g47280 | disease resistance protein (NBS-LRR class), putative domain signature NBS- |
| | LRR exists, suggestive of a disease resistance protein. |
| At2g47580 | small nuclear ribonucleoprotein (spliceosomal protein) U1A identical to |
| | GB: Z49991 U1snRNP-specific protein [Arabidopsis thaliana] |
| At2g18240 | integral membrane protein-related |
| At1g31300 | expressed protein similar to hypothetical protein GB: AAF24587 GI: 6692122 from |
| | [Arabidopsis thaliana] |
| At3g59530 | strictosidine synthase-related similar to strictosidine synthase [Rauvolfia |
| | serpentina][SP |
| At4g29600 | cytidine deaminase 7 |
| At1g67460 | hypothetical protein |
| At3g06560 | poly(A) polymerase-related similar to polynucleotide adenylyltransferase |
| | GB: S17875 from [Bos taurus] (Nature (1991) 353 (6341), 229-234) |
| At2g42030 | C3HC4-type zinc finger protein family contains Pfam profile: PF00097 zinc |
| | finger, C3HC4 type (RING finger) |
| At1g22630 | auxin-regulated protein |
| At3g42600 | hypothetical protein |
| At2g29340 | short-chain dehydrogenase/reductase family protein similar to tropinone |
| | reductase-I GI: 424160 from [Datura stramonium] |
| At1g22600 | seed maturation protein PM27-related similar to seed maturation protein PM27 |
| | GI: 4836403 from [Glycine max] |
| At1g72960 | root hair defective-related similar to root hair defective 3 GI: 1839188 from |
| | [Arabidopsis thaliana] |
| At1g24530 | transducin/WD-40 repeat protein family similar to Vegetatible incompatibility |
| | protein HET-E-1 (SP: Q00808) {Podospora anserina}; contains 7 WD-40 |
| | repeats (PF00400) |
| At1g61370 | receptor protein kinase (IRK1)-related similar to receptor protein kinase (IRK1) |
| | GI: 836953 from [Ipomoea trifida] |
| At1g75620 | hypothetical protein |
| At4g19420 | pectinacetylesterase family contains Pfam profile: PF03283 pectinacetylesterase |
| At5g27150 | sodium proton exchanger (NHX1) identical to Na+/H+ exchanger [Arabidopsis |
| | thaliana] gi |
| At2g06005 | expressed protein |
| At2g44170 | N-myristoyltransferase-related |
| At3g63240 | endonuclease/exonuclease/phosphatase family similar to inositol polyphosphate |
| | 5-phosphatase I (GI: 10444261) and II (GI: 10444263) [Arabidopsis thaliana]; |
| | contains Pfam profile PF03372: Endonuclease/Exonuclease/phosphatase family |
| At3g25890 | AP2 domain transcription factor, putative |
| At3g62190 | DnaJ protein family similar to SP |
| At4g04840 | expressed protein similar to transcriptional regulator |
| At4g35540 | hypothetical protein transcription factor IIIB chain BRF1, |
| | Saccharomyce scerevisiae, PIR2: A44072 |
| At4g28000 | hypothetical protein MSP1, Saccharomyces cerevisiae, PIR2: A49506 |
| At4g01760 | CHP-rich zinc finger protein, putative similar to T15B16.10 similar to A. thaliana |
| | CHP-rich proteins encoded by T10M13, GenBank accession number AF001308 |
| At5g52850 | pentatricopeptide (PPR) repeat-containing protein contains |
| | INTERPRO: IPR002885 PPR repeats |
| At5g15040 | hypothetical protein predicted proteins, Arabidopsis thaliana |
| At5g50870 | ubiquitin-conjugating enzyme, putative strong similarity to ubiquitin conjugating |
| | enzyme [Lycopersicon esculentum] GI: 886679; contains Pfam profile PF00179: |
| | Ubiquitin-conjugating enzyme |
| At5g55430 | hypothetical protein |
| At5g06340 | diadenosine 5′,5′″-P1,P4-tetraphosphate hydrolase, putative similar to |
| | diadenosine 5′,5′″-P1,P4-tetraphosphate hydrolase from [Lupinus angustifolius] |
| | GI: 1888557, [Hordeum vulgare subsp. vulgare] GI: 2564253; contains Pfam |
| | profile PF00293: NUDIX domai? |
| At4g37020 | expressed protein |
| At5g43380 | serine/threonine protein phosphatase type on(TOPP7) |
| At4g07720 | hypothetical protein |
| At2g02840 | hypothetical protein |
| At1g67150 | hypothetical protein |
| At1g55830 | expressed protein |
| At1g21480 | Exostosin family contains Pfam profile: PF03016 Exostosin family |
| At1g71080 | expressed protein |
| At2g11200 | F-box protein family |
| At2g38270 | expressed protein |
| At2g05400 | expressed protein |
| At2g23470 | expressed protein |
| At3g30380 | hypothetical protein contains Pfam profile: PF00561 alpha/beta hydrolase fold |
| At3g17850 | protein kinase, putative similar to IRE (incomplete root hair elongation) |
| | [Arabidopsis thaliana] gi |
| At3g29190 | terpene synthase/cyclase family contains Pfam profile: PF01397 terpene |
| | synthase family |
| At4g21840 | expressed protein CGI-131 protein, Homo sapiens, AF151889 |
| At5g57220 | cytochrome P450, putative similar to Cytochrome P450 (SP: O65790) |
| | [Arabidopsis thaliana]; Cytochrome P450 (GI: 7415996) [Lotus japonicus] |
| At5g17770 | NADH-cytochrome b5 reductase identical to NADH-cytochrome b5 reductase |
| | [Arabidopsis thaliana] GI: 4240116 |
| At5g49430 | transducin/WD-40 repeat protein family similar to WD-repeat protein 9 |
| | (SP: Q9NSI6) {Homo sapiens}; contains Pfam PF00400: WD domain, G-beta |
| | repeat (4 copies) |
| At5g59640 | serine/threonine-specific protein kinase - like putative protein serine/threonine |
| | kinase, Sorghum bicolor, EMBL: SBRLK1 |
| At5g06270 | B-type cyclin-related similar to B-type cyclin GI: 849074 from [Nicotiana |
| | tabacum] |
| At5g65070 | MADS-box protein |
| At5g01780 | oxidoreductase, 2OG-Fe(II) oxygenase family low similarity to alkB protein - |
| | Escherichia coli, PIR: BVECKB, alkB [Caulobacter crescentus][GI: 2055386]; |
| | contains Pfam domain PF03171 2OG-Fe(II) oxygenase superfamily |
| At5g15370 | hypothetical protein |
| At5g42910 | ABA-responsive element binding protein, putative |
| At4g34060 | hypothetical protein |
| At3g42480 | hypothetical protein hypothetical proteins - Arabidopsis thaliana |
| At4g24530 | PsRT17-1 like protein PsRT17-1, Pisum sativum (pea), PATX: G1778376 |
| At2g27280 | hypothetical protein |
| At1g22720 | WAK-like kinase (WLK) contains similarity to serine/threonine kinase gb |
| At4g04400 | hypothetical protein contains Pfam profile PF03384: Drosophila protein of |
| | unknown function, DUF287 |
| At2g46740 | FAD-linked oxidoreductase family strong similarity to At1g32300, At5g56490, |
| | At2g46750, At2g46760; contains PF01565: FAD binding domain |
| At1g62630 | disease resistance protein (CC-NBS-LRR class), putative domain signature CC- |
| | NBS-LRR exists, suggestive of a disease resistance protein. |
| At2g13900 | CHP-rich zinc finger protein, putative |
| At4g28630 | ABC transporter family protein identical to half-molecule ABC transporter ATM1 |
| | GI: 9964117 from [Arabidopsis thaliana] |
| At1g31320 | lateral organ boundaries (LOB) domain family similar to lateral organ boundaries |
| | (LOB) domain-containing proteins from Arabidopsis thaliana |
| At1g24200 | hypothetical protein similar to hypothetical protein, GB: AAB61107 |
| At1g04070 | expressed protein Contains similarity to hypothetical mitochondrial import |
| | receptor subunit gb Z98597 from S. pombe. ESTs gb |
| At1g72810 | threonine synthase, putative strong similarity to SP |
| At1g10522 | expressed protein |
| At1g78100 | F-box protein family contains F-box domain Pfam: PF00646 |
| At1g68720 | deaminase-related similar to cytidine/deoxycytidylate deaminase family protein |
| | GB: AAF73539 GI: 8163170 from [Chlamydia muridarum] |
| At1g11220 | expressed protein contains similarity to cotton fiber expressed protein |
| | GB: AAC33276 from [Gossypium hirsutum] |
| At1g73970 | expressed protein |
| At1g66840 | hypothetical protein |
| At1g01650 | expressed protein |
| At2g26310 | expressed protein and grail |
| At2g22290 | GTP-binding protein, putative similar to GTP-binding protein GI: 550072 from |
| | [Homo sapiens] |
| At2g45280 | RAD51C DNA repair protein-related |
| At3g05460 | expressed protein |
| At3g52900 | expressed protein chromosome assembly protein homolog, Aquifex aeolicus, |
| | PIR: B70356 |
| At4g04360 | hypothetical protein |
| At4g26850 | expressed protein |
| At2g43970 | VirF-interacting protein FIP1 |
| At5g44870 | disease resistance protein (TIR-NBS-LRR class), putative domain signature TIR- |
| | NBS-LRR exists, suggestive of a disease resistance protein. |
| At5g47550 | expressed protein similar to unknown protein (pir |
| At5g39360 | expressed protein predicted proteins, Arabidopsis thaliana |
| At3g19980 | protein phosphatase similar to serine/threonine protein phosphatase GB: Z47076 |
| | GI: 1143510 [Malus domestica] |
| At3g42220 | transposase - like protein putative transposase protein Shooter, Zea mays, |
| | EMBL: AF136220 |
| At3g10270 | DNA topoisomerase [ATP-hydrolyzing] (DNA topoisomerase II/DNA gyrase), |
| | putative similar to SP |
| At1g24090 | RNase H domain-containing protein very low similarity to GAG-POL precursor |
| | [Oryza sativa (japonica cultivar-group)] GI: 5902445; contains Pfam profiles |
| | PF00075: RNase H, PF04134: Protein of unknown function, DUF393 |
| At1g08260 | DNA polymerase epsilon catalytic subunit-related similar to DNA polymerase |
| | epsilon catalytic subunit GI: 5565875 from [Mus musculus] |
| At1g69970 | CLE26, putative CLAVATA3/ESR-Related 26 (CLE26); |
| At4g21680 | peptide transporter - like protein peptide transporter (ptr1) - Hordeum |
| | vulgare, AF023472 |
| At5g55540 | expressed protein similar to unknown protein (gb |
| At2g33550 | expressed protein |
| At2g28520 | vacuolar proton-ATPase subunit-related |
| At2g46250 | expressed protein and genefinder |
| At2g37650 | scarecrow transcription factor family |
| At2g42230 | expressed protein |
| At2g34190 | membrane transporter-related |
| At3g43180 | C3HC4-type zinc finger protein family contains Pfam profile: PF00097 zinc |
| | finger, C3HC4 type (RING finger) |
| At3g52620 | hypothetical protein phosphate actyltransferase, Staphylococcus aureus, |
| | EMBL: SAU271496 |
| At3g27390 | expressed protein |
| At3g58710 | WRKY family transcription factor contains Pfam profile: PF03106 WRKY DNA - |
| | binding domain |
| At3g09950 | hypothetical protein |
| At3g21230 | 4-coumarate: CoA ligase (4-coumaroyl-CoA synthase) (4CL), putative similar to |
| | 4CL2 [gi: 12229665] and 4CL1 [gi: 12229649] from [Arabidopsis thaliana], 4CL1 |
| | [gi: 12229631] from Nicotiana tabacum |
| At4g29270 | acid phosphatase-related protein acid phosphatase-1 (EC 3.1.3.— ) - |
| | Lycopersicon esculentum, PIR2: T06587 |
| At5g12870 | myb family transcription factor contains PFAM profile: myb DNA binding domain |
| | PF00249 |
| At5g45170 | expressed protein similar to unknown protein (pir |
| At5g18260 | expressed protein |
| At5g39380 | expressed protein predicted protein, Arabidopsis thaliana |
| At5g66560 | phototropic response protein family contains NPH3 family domain, |
| | Pfam: PF03000 |
| At5g15470 | glycosyltransferase family 8 contains Pfam profile: PF01501 glycosyl transferase |
| | family 8 |
| At5g38960 | germin-like protein, putative similar to germin-like protein subfamily 1 member 8 |
| | [SP |
| At5g41460 | fringe-related protein strong similarity to unknown protein (pir |
| At3g46170 | short-chain dehydrogenase/reductase family protein contains similarity to 3- |
| | oxoacyl-[acyl-carrier protein] reductase SP: P51831 from [Bacillus subtilis] |
| At4g22070 | WRKY family transcription factor identical to WRKY transcription factor 31 |
| | (WRKY31) GI: 15990589 from [Arabidopsis thaliana] |
| At5g06390 | expressed protein strong similarity to unknown protein (gb |
| At2g32430 | galactosyltransferase family contains Pfam profile: PF01762 |
| | galactosyltransferase |
| At1g71240 | expressed protein |
| At3g23980 | expressed protein |
| At1g03060 | putataive transport protein Similar to gb |
| At4g34090 | expressed protein |
| At1g55740 | glycosyl hydrolase family 36 similar to seed imbibition protein GB: AAA32975 |
| | GI: 167100 from [Hordeum vulgare] |
| At1g16190 | DNA repair protein RAD23, putative similar to DNA repair by nucleotide excision |
| | (NER) RAD23 protein, isoform II GI: 1914685 from [Daucus carota] |
| At1g12030 | hypothetical protein |
| At1g64355 | expressed protein |
| At1g47350 | hypothetical protein similar to hypothetical protein GB: AAD22292 GI: 6598654 |
| | from [Arabidopsis thaliana] |
| At5g66020 | hypothetical protein non-consensus AT donor splice site at exon 7, TA donor |
| | splice site at exon 10, AT acceptor splice at exon 13, strong similarity to |
| | unknown protein (emb |
| At2g43420 | 3-beta hydroxysteroid dehydrogenase/isomerase family contains Pfam profile |
| | PF01073 3-beta hydroxysteroid dehydrogenase/isomerase domain; similar to |
| | NAD(P)-dependent steroid dehydrogenase from Homo sapiens [SP |
| At3g56980 | bHLH protein family NULL |
| At3g52200 | dihydrolipoamide S-acetyltransferase (LTA3); nuclear gene encoding |
| | mitochondrial protein annotation temporarily based on supporting cDNA gi |
| At3g10400 | RNA recognition motif (RRM) - containing protein low similarity to splicing factor |
| | SC35 [Arabidopsis thaliana] GI: 9843653; contains InterPro entry IPR000504: |
| | RNA-binding region RNP-1 (RNA recognition motif) (RRM) |
| At3g25960 | pyruvate kinase, putative similar to pyruvate kinase, cytosolic isozyme [Nicotiana |
| | tabacum] SWISS-PROT: Q42954 |
| At3g07990 | serine carboxypeptidase-related similar to serine carboxypeptidase II (CP-MII) |
| | GB: CAA70815 [Hordeum vulgare] |
| At3g06270 | protein phosphatase 2C (PP2C), putative similar to protein phosphatase-2C |
| | (PP2C) GB: AAC36699 [Mesembryanthemum crystallinum]; contains Pfam |
| | profile: PF00481 protein phosphatase 2C |
| At3g05200 | RING-H2 zinc finger protein ATL6-related similar to GB: AAD33584 from |
| | [Arabidopsis thaliana] |
| At3g61600 | POZ domain protein family contains Pfam PF00651: BTB/POZ domain; contains |
| | Interpro IPR000210/PS50097: BTBB/POZ domain; similar to POZ/BTB |
| | containing-protein AtPOB1 (GI: 12006855) [Arabidopsis thaliana]; similar to |
| | actinfilin (GI: 21667852) [Rattus norv? |
| At4g10300 | expressed protein predicted protein, Arabidopsis thaliana |
| At5g37790 | protein kinase family contains protein kinase domain, Pfam: PF00069 |
| At5g61850 | LFY floral meristem identity control protein |
| At5g44040 | expressed protein similar to unknown protein (gb |
| At5g40580 | 20S proteasome beta subunit B (PBB2) |
| At4g33800 | expressed protein |
| At5g41060 | DHHC-type zinc finger domain-containing protein contains Pfam profile |
| | PF01529: DHHC zinc finger domain |
| At3g45940 | glycosyl hydrolase family 31 similar to alpha-xylosidase precursor GI: 4163997 |
| | from [Arabidopsis thaliana] |
| At5g66710 | protein kinase, putative similar to protein kinase ATN1 GP |
| At2g45900 | expressed protein |
| At1g68280 | hypothetical protein |
| At4g01730 | DHHC-type zinc finger domain-containing protein contains Pfam profile |
| | PF01529: DHHC zinc finger domain |
| At1g23460 | polygalacturonase, putative similar to polygalacturonase GB: BAA88472 |
| | GI: 6624205 from (Cucumis sativus) |
| At1g21620 | pumilio-family RNA-binding protein, putative similar to hypothetical protein |
| | GB: AAD41414 GI: 5263312 from (Arabidopsis thaliana) |
| At1g16640 | transcriptional factor B3 family low similarity to reproductive meristem protein 1 |
| | [Arabidopsis thaliana] GI: 13604227; contains Pfam profile PF02362: B3 DNA |
| | binding domain |
| At1g18460 | lipase family similar to triacylglycerol lipase, gastric precursor (EC 3.1.1.3) |
| | {Canis familiaris} [SP |
| At1g49890 | expressed protein |
| At4g08680 | MuDR-A transposon protein-related similar to Z. mays MuDR-A protein |
| At5g41490 | hypothetical protein strong similarity to unknown protein (gb |
| At2g02790 | hypothetical protein |
| At2g39870 | expressed protein |
| At2g41040 | expressed protein |
| At2g15050 | lipid transfer protein, putative similar to SP |
| At2g28250 | protein kinase family contains protein kinase domain, Pfam: PF00069 |
| At3g20720 | expressed protein |
| At3g01900 | cytochrome P450 family similar to Cytochrome P450 94A1 (P450-dependent |
| | fatty acid omega-hydroxylase) (SP: O81117) {Vicia sativa}; contains Pfam |
| | profile: PF00067 cytochrome P450 |
| At3g62420 | bZIP family transcription factor similar to common plant regulatory factor 6 |
| | GI: 9650826 from [Petroselinum crispum] |
| At3g20030 | F-box protein family contains F-box domain Pfam: PF00646 |
| At4g33840 | glycosyl hydrolase family 10 xylan endohydrolase isoenzyme X-I, Hordeum |
| | vulgare, PID: g1813595 |
| At4g26660 | expressed protein probable kinesin - Arabidopsis thaliana, Pir2: H71402 |
| At4g25510 | hypothetical protein |
| At4g27980 | expressed protein |
| At4g37710 | expressed protein predicted protein, Arabidopsis thaliana |
| At5g51210 | oleosin |
| At5g03180 | C3HC4-type zinc finger protein family various predicted proteins, Arabidopsis |
| | thaliana; contains Pfam profile PF00097: Zinc finger, C3HC4 type (RING finger) |
| At5g59940 | CHP-rich zinc finger protein, putative large number of predicted zinc finger |
| | proteins, Arabidopsis thaliana, Homo sapiens and others |
| At4g11720 | hypothetical protein histidine-rich glycoprotein precursor, Plasmodium lophurae, |
| | PIR1: KGZQHL |
| At3g26250 | CHP-rich zinc finger protein, putative |
| At2g24950 | hypothetical protein contains Pfam profile PF03080: Arabidopsis proteins of |
| | unknown function |
| At3g16390 | jacalin lectin family similar to myrosinase-binding protein homolog [Arabidopsis |
| | thaliana] GI: 2997767, epithiospecifier [Arabidopsis thaliana] GI: 16118845; |
| | contains Pfam profiles PF01419 jacalin-like lectin family, PF01344 Kelch motif |
| At2g35330 | expressed protein |
| At2g17200 | ubiquitin protein-related |
| At1g47840 | hexokinase-related similar to hexokinase 2 GB: AAB49911 GI: 1899025 from |
| | [Arabidopsis thaliana] |
| At1g28430 | cytochrome P450, putative similar to cytochrome P450 (CYP93A1) GI: 1435059 |
| | from [Glycine max] |
| At1g65870 | disease resistance response protein-related/dirigent protein-related similar to |
| | dirigent protein [Forsythia X intermedia] gi |
| At1g55880 | pyridoxal-5′-phosphate-dependent enzyme, beta family similar to SP |
| At1g17250 | leucine rich repeat protein family contains leucine rich-repeat (LRR) domains |
| | Pfam: PF00560, INTERPRO: IPR001611; similar to Hcr2-0B [Lycopersicon |
| | esculentum] gi |
| At5g21070 | expressed protein predicted protein - Oryza sativa - TREMBL: AP001072_3 |
| At2g38780 | expressed protein |
| At2g13840 | expressed protein |
| At2g48100 | exonuclease-related annotation temporarily based on supporting cDNA gi |
| At2g37040 | phenylalanine ammonia lyase (PAL1) nearly identical to SP |
| At3g04460 | expressed protein similar to peroxisomal biogenesis factor 12 GB: NP_000277 |
| | [Homo sapiens] |
| At3g13140 | hypothetical protein |
| At3g43720 | protease inhibitor/seed storage/lipid transfer protein (LTP) family contains Pfam |
| | protease inhibitor/seed storage/LTP family domain PF00234 |
| At4g39220 | AtRer1A |
| At4g12240 | hypothetical proteins |
| At4g07770 | pseudogene, similar to L1 repeat, Tf subfamily, member 30 (LINE-element) [Mus |
| | musculus] (GB: NP_038605) |
| At4g14920 | PHD finger transcription factor, putative |
| At5g46280 | DNA replication licensing factor, putative similar to SP |
| At5g43340 | inorganic phosphate transporter identical to inorganic phosphate transporter |
| | [Arabidopsis thaliana] GI: 3869190 |
| At5g42250 | alcohol dehydrogenase (ADH), putative similar to alcohol dehydrogenase ADH |
| | GI: 7705214 from [Lycopersicon esculentum]; contains Pfam zinc-binding |
| | dehydrogenase domain PF00107 |
| At5g58540 | expressed protein serine/threonine-specific protein kinase NPK15, Nicotiana |
| | tabacum, PIR: S52578 |
| At5g52510 | scarecrow-like transcription factor 8 (SCL8) |
| At2g43640 | signal recognition particle protein 14 kD, ATSRP14-related |
| At4g03870 | pseudogene, putative transposon protein similar to MuDR transposon |
| At5g49270 | expressed protein contains similarity to phytochelatin synthetase |
| At5g61110 | hypothetical protein |
| At5g07810 | SNF2 domain/helicase domain-containing protein similar to HepA-related protein |
| | HARP [Homo sapiens] GI: 6693791; contains Pfam profiles PF00271: Helicase |
| | conserved C-terminal domain, PF00176: SNF2 family N-terminal domain, |
| | PF01844: HNH endonuclease |
| At2g32920 | protein disulfide isomerase family similar to SP |
| At2g29920 | hypothetical protein |
| At1g37045 | an Arabidopsis thaliana hypothetical protein, which contains similarity to |
| | retrotransposon Athila (GB: AF076275)-related temporary automated functional |
| | assignment |
| At5g61700 | ABC transporter family protein ABC family transporter, Entamoeba histolytica, |
| | EMBL: EH058 |
| At1g56720 | protein kinase family contains protein kinase domain, Pfam: PF00069 |
| At1g49160 | protein kinase family contains protein kinase domain, Pfam: PF00069 |
| At1g02770 | expressed protein similar to Hypothetical protein GB: AAF02890 GI: 6056426 |
| | from (Arabidopsis thaliana) |
| At5g64960 | Cyclin-dependent kinase C; 2 |
| At1g35460 | bHLH protein similar to GI: 6166283 from [Pinus taeda] |
| At2g44080 | expressed protein |
| At2g30490 | cytochrome P450 73/trans-cinnamate 4-monooxygenase/cinnamate-4- |
| | hydroxylase (CYP73) (C4H) identical to SP |
| At2g42070 | MutT/nudix family protein similar to SP |
| At3g50170 | hypothetical protein various predicted genes, Arabidopsis thaliana and Oryza |
| | sativa |
| At3g59870 | expressed protein hypothetical protein F6E13.7 - Arabidopsis thaliana, |
| | PIR: T00674 |
| At3g54680 | proteophosphoglycan-related contains similarity to proteophosphoglycan |
| | [Leishmania major] gi |
| At3g61270 | expressed protein several hypothetical proteins - Arabidopsis thaliana |
| At3g02290 | C3HC4-type zinc finger protein family contains zinc finger motif, C3HC4 type |
| | (RING finger) |
| At3g14500 | hypothetical protein |
| At4g12700 | expressed protein |
| At4g24230 | expressed protein acyl-CoA binding protein - Arabidopsis thaliana, PID: g4128197 |
| At4g27340 | expressed protein met-10+ protein, Neurospora crassa, PIR2: S46697 |
| At4g38225 | expressed protein |
| At4g31280 | hypothetical protein |
| At5g50280 | pentatricopeptide (PPR) repeat-containing protein contains Pfam profile |
| | PF01535: PPR repeat |
| At5g53940 | zinc-binding protein-related |
| At1g16400 | cytochrome P450 family similar to gb |
| At5g48290 | heavy-metal-associated domain-containing protein strong similarity to |
| | farnesylated proteins ATFP4 [GI: 4097549] and ATFP5 [GI: 4097551]; contains |
| | Pfam profile PF00403: Heavy-metal-associated domain |
| At1g21245 | wall-associated kinase 3-related temporary automated functional assignment |
| At5g60140 | transcriptional factor B3 family contains Pfam profile PF02362: B3 DNA binding |
| | domain |
| At1g22090 | expressed protein |
| At1g04970 | expressed protein |
| At1g55750 | expressed protein |
| At1g59760 | ATP-dependent RNA helicase, putative similar to SP |
| At2g39300 | hypothetical protein |
| At2g37340 | splicing factor RSZ33, putative nearly identical to splicing factor RSZ33 |
| | [Arabidopsis thaliana] GI: 9843663 |
| At3g10210 | expressed protein similar to putative protein GB: CAA20045 [Arabidopsis |
| | thaliana] |
| At3g05430 | PWWP domain protein contains Pfam profile: PF00855 PWWP domain |
| At3g48600 | expressed protein |
| At4g14830 | expressed protein |
| At5g19780 | tubulin alpha-3/alpha-5 chain (TUA5) nearly identical to SP |
| At5g55050 | GDSL-motif lipase/hydrolase protein similar to family II lipases EXL3 |
| | GI: 15054386, EXL1 GI: 15054382, EXL2 GI: 15054384 from [Arabidopsis |
| | thaliana]; contains Pfam profile PF00657: GDSL-like Lipase/Acylhydrolase |
| At5g39260 | expansin, putative (EXP21) similar to alpha-expansin GI: 6573157 from |
| | [Regnellidium diphyllum]; alpha-expansin gene family, PMID: 11641069 |
| At5g51550 | expressed protein similar to unknown protein (gb |
| At4g39780 | AP2 domain transcription factor, putative similar to AP2 domain containing |
| | protein RAP2.4, Arabidopsis thaliana |
| At1g10650 | conserved hypothetical protein |
| At3g12430 | hypothetical protein |
| At5g48410 | glutamate receptor family (GLR1.3) plant glutamate receptor family, |
| | PMID: 11379626 |
| At1g24220 | hypothetical protein |
| At1g80960 | expressed protein |
| At1g64460 | phosphatidylinositol 3- and 4-kinase family contains Pfam profile PF00454: |
| | Phosphatidylinositol 3- and 4-kinase |
| At1g75980 | expressed protein |
| At1g33800 | expressed protein |
| At2g27950 | expressed protein |
| At2g44830 | protein kinase putative similar to protein kinase PVPK-1 [Phaseolus vulgaris] |
| | SWISS-PROT: P15792 |
| At3g43200 | pseudogene, putative protein predicted proteins, Arabidopsis thaliana |
| At3g10970 | haloacid dehalogenase-like hydrolase family low similarity to genetic modifier |
| | [Zea mays] GI: 10444400; contains InterPro accession IPR005834: Haloacid |
| | dehalogenase-like hydrolase |
| At3g51920 | calmodulin 9 identical to calmodulin 9 GI: 5825602 from [Arabidopsis thaliana] |
| At4g28980 | cdk-activating kinase 1At identical to Cdk-activating kinase 1At [Arabidopsis |
| | thaliana] gi |
| At4g16690 | esterase/lipase/thioesterase family similar to ethylene-induced esterase [Citrus |
| | sinensis] GI: 14279437, polyneuridine aldehyde esterase [Rauvolfia serpentina] |
| | GI: 6651393, SP |
| At5g35180 | expressed protein |
| At5g57730 | hypothetical protein |
| At5g09560 | KH domain protein various predicted RNA binding proteins, Arabidopsis thaliana |
| At5g59770 | expressed protein protein tyrosine phosphatase-like protein, PTPLB, Mus |
| | musculus, EMBL: AF169286 |
| At2g31570 | glutathione peroxidase, putative |
| At2g26190 | expressed protein |
| At3g10860 | ubiquinol-cytochrome C reductase complex ubiquinone-binding protein (QP-C) - |
| | related similar to ubiquinol-cytochrome C reductase complex ubiquinone- |
| | binding protein (QP-C) GB: P46269 [Solanum tuberosum] |
| At1g49080 | pseudogene, putative transposon protein similar to Antirrhinum majus TNP2 |
| | protein gb |
| At1g28300 | transcriptional factor B3 protein leafy cotyledon 2 nearly identical to LEAFY |
| | COTYLEDON 2 [Arabidopsis thaliana] GI: 15987516; contains Pfam profile |
| | PF02362: B3 DNA binding domain |
| At1g36310 | expressed protein |
| At1g47860 | reverse transcriptase-related low similarity to reverse transcriptase [Arabidopsis |
| | thaliana] GI: 976278; contains Pfam profiles PF00078: Reverse transcriptase |
| | (RNA-dependent DNA polymerase), PF00096: Zinc finger, C2H2 type, |
| | PF03727: Hexokinase |
| At1g61410 | expressed protein similar to putative double strand break repair protein |
| | GI: 9651817 from [Arabidopsis thaliana] |
| At1g13940 | expressed protein identical to hypothetical protein GB: AAD39280 GI: 5080770 |
| | from [Arabidopsis thaliana] |
| At1g65650 | expressed protein similar to ubiquitin C-terminal hydrolase-like protein |
| | GI: 9759113 from [Arabidopsis thaliana] |
| At1g31150 | expressed protein EST gb |
| At1g55550 | kinesin-related protein Similar to Kinesin proteins; Contains kinesin motor |
| | domain protein motif and kinesin heavy chain signature motif |
| At2g23890 | expressed protein and genefinder |
| At2g07030 | Mutator-related transposase similar to MURA transposase of maize Mutator |
| | transposon |
| At2g14810 | hypothetical protein |
| At3g46470 | hypothetical protein |
| At3g06400 | DNA-dependent ATPase, putative similar to DNA-dependent ATPase SNF2H |
| | [Mus musculus] GI: 14028669; contains Pfam profiles PF00271: Helicase |
| | conserved C-terminal domain, PF00176: SNF2 family N-terminal domain, |
| | PF00249: Myb-like DNA-binding domain |
| At3g43590 | expressed protein hexamer-binding protein HEXBP - Leishmania major, |
| | PIR: A47156 |
| At3g16360 | two-component phosphorelay mediator-related similar to two-component |
| | phosphorelay mediators (ATHP1-3) GB: BAA37110, GB: BAA37111, |
| | GB: BAA37112 [Arabidopsis thaliana] |
| At4g32620 | expressed protein predicted protein T10M13.8, Arabidopsis thaliana |
| At5g56340 | expressed protein similar to unknown protein (pir |
| At5g50100 | expressed protein similar to unknown protein (pir |
| At5g55690 | MADS-box protein |
| At5g09840 | expressed protein similar to unknown protein (emb |
| At5g42130 | mitochondrial carrier protein family contains Pfam profile: PF00153 mitochondrial |
| | carrier protein |
| At5g40220 | MADS-box protein MADS-box protein, Arabidopsis thaliana, EMBL: ATY12776 |
| At3g44010 | 40S ribosomal protein S29 (RPS29B) ribosomal protein S29, rat, PIR: S30298 |
| At3g45190 | expressed protein hypothetical protein At2g28360 - Arabidopsis thaliana, |
| | EMBL: AAD20690 |
| At5g44410 | FAD-linked oxidoreductase family similar to SP |
| At1g18120 | pseudogene, putative myrosinase-associated protein |
| At5g39000 | protein kinase family contains protein kinase domain, Pfam: PF00069 |
| At4g03970 | Ulp1 protease family contains Pfam profile PF02902: Ulp1 protease family, C- |
| | terminal catalytic domain; similar to At5g28170, At1g35110, At1g44880, |
| | At3g42530, At4g19320, At5g36020, At3g43010, At2g10350 |
| At1g56420 | hypothetical protein |
| At1g61680 | terpene synthase/cyclase family similar to 1,8-cineole synthase |
| | [GI: 3309117][Salvia officinalis]; contains Pfam profile: PF01397 terpene |
| | synthase family |
| At1g06520 | phospholipid/glycerol acyltransferase family contains Pfam profile PF01553: |
| | Acyltransferase |
| At1g54550 | F-box protein family contains Pfam: PF00646 F-box domain; contains TIGRFAM |
| | TIGR01640: F-box protein interaction domain |
| At2g01340 | expressed protein |
| At2g44130 | Kelch repeat containing F-box protein family very low similarity to SP |
| At2g24670 | hypothetical protein |
| At3g23080 | expressed protein C-term similar to phosphatidylcholine transfer protein |
| | GB: AAF08345 [Homo sapiens] |
| At3g09310 | alpha-hemolysin-related similar to alpha-hemolysin GB: AAB81225 [Aeromonas |
| | hydrophila] |
| At3g28430 | expressed protein GC donor splice site at exon 16 |
| At3g23670 | phragmoplast-associated kinesin-related protein, putative similar to kinesin like |
| | protein GB: CAB10194 from [Arabidopsis thaliana] |
| At4g19350 | expressed protein |
| At4g30300 | ABC transporter family protein ribonuclease L inhibitor - Mus |
| | musculus, PIR2: JC6555 |
| At4g00760 | expressed protein |
| At4g28180 | hypothetical protein |
| At4g18320 | hypothetical protein |
| At4g18820 | expressed protein DNA polymerase III holoenzyme tau subunit, Thermus |
| | thermophilus, gb: AF025391 |
| At5g12970 | C2 domain-containing protein contains INTERPRO: IPR000008 C2 domain |
| At5g66350 | zinc finger protein SHI-related |
| At5g13080 | WRKY family transcription factor WRKY DNA binding protein - Solanum |
| | tuberosum, EMBL: AJ278507 |
| At5g22550 | expressed protein strong similarity to unknown protein (emb |
| At5g39630 | SNARE protein AtMEMB11 v-SNARE AtVTI1a, Arabidopsis thaliana, |
| | EMBL: AF114750 |
| At3g51090 | expressed protein hypothetical protein F16F14.4 - Arabidopsis thaliana: |
| | EMBL: AC007047 |
| At5g43270 | squamosa promoter binding protein-related 2 (emb |
| At1g54760 | MADS-box protein similar to MADS-box transcription factor GI: 4837612 from |
| | [Antirrhinum majus] |
| At5g15650 | reversibly glycosylated polypeptide-3 |
| At3g19800 | expressed protein |
| At2g18860 | expressed protein |
| At2g03260 | expressed protein |
| At3g05240 | pentatricopeptide (PPR) repeat-containing protein contains Pfam profile |
| | PF01535: PPR repeat |
| At1g05660 | polygalacturonase, putative similar to GB: AAC23398 |
| At1g48670 | Nt-gh3 deduced protein-related similar to Nt-gh3 deduced protein GI: 4887010 |
| | from [Nicotiana tabacum] |
| At1g31850 | dehydration-induced protein, putative strong similarity to early-responsive to |
| | dehydration stress ERD3 protein [Arabidopsis thaliana] GI: 15320410; contains |
| | Pfam profile PF03141: Putative methyltransferase |
| At1g08500 | plastocyanin-like domain containing protein |
| At1g59640 | bHLH protein |
| At1g68500 | expressed protein |
| At2g27240 | expressed protein contains Pfam profile PF01027: Uncharacterized protein |
| | family UPF0005 |
| At2g07240 | Ulp1 protease family contains Pfam profile PF02902: Ulp1 protease family, C- |
| | terminal catalytic domain |
| At3g22790 | expressed protein similar to centromere protein homolog GB: CAB10255 from |
| | [Arabidopsis thaliana] |
| At3g16010 | pentatricopeptide (PPR) repeat-containing protein contains Pfam profile |
| | PF01535: PPR repeat |
| At3g57540 | expressed protein putative DNA binding protein - Arabidopsis thaliana, |
| | TREMBL: ATAC2339_3 |
| At4g12270 | copper amine oxidase like protein (fragment1) copper amine oxidase - Cicer |
| | arietinum, PID: e1335964 |
| At4g04950 | thioredoxin family similar to PKCq-interacting protein PICOT from [Mus |
| | musculus] GI: 6840949, [Rattus norvegicus] GI: 6840951; contains Pfam profile |
| | PF00085: Thioredoxin |
| At4g20280 | expressed protein transcription initiation factor IID beta chain, fruit fly, |
| | Pir2: B49453 |
| At4g02280 | sucrose synthase (UDP-glucose-fructose glucosyltransferase/sucrose-UDP |
| | glucosyltransferase), putative strong similarity to sucrose synthase GI: 6682841 |
| | from [Citrus unshiu] |
| At5g58787 | C3HC4-type zinc finger protein family similar to MTD2 [Medicago truncatula] |
| | GI: 9294812; contains Pfam profile PF00097: Zinc finger, C3HC4 type (RING |
| | finger) |
| At5g51480 | pectinesterase (pectin methylesterase) family similar to pectinesterase |
| | GB: CAB08077 GI: 1944575 from [Lycopersicon esculentum]; contains Pfam |
| | profile: PF00394 Multicopper oxidase; similar to pollen-specific protein |
| At5g61650 | cyclin family similar to cyclin 2 [Trypanosoma brucei] GI: 7339572, cyclin 6 |
| | [Trypanosoma cruzi] GI: 12005317; contains Pfam profile PF00134: Cyclin, N- |
| | terminal domain |
| At3g53080 | expressed protein BETA-GALACTOSIDASE PRECURSOR. Lycopersicon |
| | esculentum, gb: P48980 |
| At4g35040 | bZIP protein |
| At1g55210 | disease resistance response protein-related/dirigent protein-related smimilar to |
| | dirigent protein [Thuja plicata] gi |
| At1g51430 | expressed protein |
| At4g39770 | trehalose-6-phosphate phosphatase, putative similar to trehalose-6-phosphate |
| | phosphatase (AtTPPB) [Arabidopsis thaliana] GI: 2944180; contains Pfam profile |
| | PF02358: Trehalose-phosphatase |
| At4g13760 | polygalacturonase, putative polygalacturonase, Zea mays, PIR2: S30067 |
| At4g00930 | expressed protein |
| At2g14470 | hypothetical protein low similarity to SP |
| At1g77250 | hypothetical protein |
| At1g52610 | mutator-related transposase similar to mutator-like transposase GI: 5306250 |
| | from [Arabidopsis thaliana] |
| At5g61710 | hypothetical protein predicted protein, Arabidopsis thaliana |
| At1g55110 | zinc finger protein-related similar to zinc finger protein GI: 8843731 from |
| | [Arabidopsis thaliana] |
| At1g05260 | peroxidase, putative similar to peroxidase precursor [Arabidopsis thaliana] gi |
| At5g27000 | kinesin-related protein non-consensus AT donor splice site at exon 12; non- |
| | consensus AC acceptor splice site at exon 13 |
| At2g40690 | glycerol-3-phosphate dehydrogenase |
| At2g26420 | phosphatidylinositol-4-phosphate 5-kinase-related |
| At3g28210 | zinc finger protein (PMZ)-related identical to putative zinc finger protein (PMZ) |
| | GB: AAD37511 GI: 5006473 [Arabidopsis thaliana] |
| At3g17880 | thioredoxin, putative similar to SP |
| At4g07940 | hypothetical protein |
| At3g61400 | 2-oxoglutarate-dependent dioxygenase, putative similar to 2A6 (GI: 599622) and |
| | tomato ethylene synthesis regulatory protein E8 (SP |
| At3g15130 | pentatricopeptide (PPR) repeat-containing protein contains |
| | INTERPRO: IPR002885 PPR repeats |
| At3g24840 | phosphatidylinositol transfer protein-related similar to SEC14 CYTOSOLIC |
| | FACTOR (PHOSPHATIDYLINOSITOL/PHOSPHATIDYLCHOLINE TRANSFER |
| | PROTEIN) GB: P46250 from [Candida albicans] (Yeast (1996) 12(11), 1097-1105) |
| At3g01710 | hypothetical protein |
| At3g01930 | expressed protein similar to nodule-specific protein Nlj70 GB: AAC39500 [Lotus |
| | japonicus] |
| At3g29635 | transferase family similar to anthocyanin 5-aromatic acyltransferase from |
| | Gentiana triflora GI: 4185599, malonyl CoA: anthocyanin 5-O-glucoside-6′″-O- |
| | malonyltransferase from Perilla frutescens GI: 17980232, Salvia splendens |
| | GI: 17980234; contains Pfam pr? |
| At4g31980 | expressed protein EREBP-4 homolog, Arabidopsis thaliana |
| At4g32910 | expressed protein |
| At4g37170 | pentatricopeptide (PPR) repeat-containing protein contains Pfam profile |
| | PF01535: PPR repeat |
| At4g33030 | UDP-sulfoquinovose synthase (sulfite: UDP-glucose sulfotransferase) (sulfolipid |
| | biosynthesis protein) (SQD1) identical to gi: 2736155 |
| At4g04330 | expressed protein |
| At5g24500 | expressed protein |
| At5g48020 | expressed protein |
| At5g54660 | expressed protein |
| At5g46160 | ribosomal protein L14p family |
| At5g06540 | pentatricopeptide (PPR) repeat-containing protein contains Pfam profile |
| | PF01535: PPR repeat |
| At5g37200 | C3HC4-type zinc finger protein family low similarity to ring-H2 finger protein |
| | RHY1a from Arabidopsis thaliana [gi: 3790593], ring finger-H2 protein from |
| | Xenopus laevis [gi: 13752371]; contains Pfam domain zinc finger, C3HC4 type |
| | (RING finger) PF00097 |
| At3g10740 | glycosyl hydrolase family 51 similar to arabinoxylan arabinofuranohydrolase |
| | isoenzyme AXAH-II from GI: 13398414 [Hordeum vulgare] |
| At4g13170 | 60S ribosomal protein L13A (RPL13aC) ribosomal protein L13a - Lupinus |
| | luteus, PID: e1237871 |
| At3g27050 | expressed protein |
| At1g60095 | jacalin lectin family contains similarity to myrosinase-binding protein homolog |
| | [Arabidopsis thaliana] GI: 2997767; |
| At3g09410 | pectinacetylesterase family similar to pectinacetylesterase precursor |
| | GB: CAA67728 [Vigna radiata]; contains Pfam profile: PF03283 |
| | pectinacetylesterase |
| At1g11655 | expressed protein |
| At1g49800 | hypothetical protein |
| At1g77610 | glucose-6-phosphate/phosphate translocator-related similar to glucose-6- |
| | phosphate/phosphate-translocators from [Mesembryanthemum crystallinum] |
| | GI: 9295277, [Solanum tuberosum] GI: 2997593, [Pisum sativum] GI: 2997591; |
| | contains Pfam profile PF00892: Integ? |
| At1g10380 | expressed protein |
| At1g28410 | expressed protein |
| At1g77350 | expressed protein |
| At1g01880 | hypothetical protein contains similarity to DNA repair endonuclease |
| | GB: AAD47568 GI: 5712619 from [Drosophila melanogaster] |
| At1g28100 | expressed protein |
| At1g35650 | Ulp1 protease family PF02902: Ulp1 protease family, C-terminal catalytic |
| | domain; similar to At1g21020, At3g26530, At1g08760, At1g08740, At2g29240 |
| At1g28560 | expressed protein |
| At2g13230 | retroelement pol polyprotein-related |
| At2g40580 | protein kinase family contains protein kinase domain, Pfam: PF00069 |
| At3g62320 | hypothetical protein hypothetical protein At2g36110 - Arabidopsis thaliana, |
| | EMBL: AC007135 |
| At3g05340 | pentatricopeptide (PPR) repeat-containing protein contains |
| | INTERPRO: IPR002885 PPR repeats |
| At3g02630 | acyl-[acyl-carrier-protein] desaturase (stearoyl-ACP desaturase), putative similar |
| | to Acyl-[acyl-carrier protein] desaturase from Sesamum indicum GI: 575942, |
| | Cucumis sativus SP |
| At3g08600 | expressed protein |
| At4g03560 | two-pore calcium channel (TPC1) identical to two-pore calcium channel (TPC1) |
| | [Arabidopsis thaliana] gi |
| At5g13680 | expressed protein similar to unknown protein (ref |
| At5g17420 | cellulose synthase, catalytic subunit (IRX3) identical to gi: 5230423 |
| At5g56850 | expressed protein similar to unknown protein (pir |
| At5g67460 | glycosyl hydrolase family 17 similar to beta-1,3-glucanase GI: 6714534 from |
| | [Salix gilgiana] |
| At5g36200 | hypothetical protein similar to unknown protein (pir |
| At5g54150 | hypothetical protein similar to unknown protein (pir |
| At5g48700 | ubiquitin family contains INTERPRO: IPR000626 ubiquitin domain |
| At5g23230 | isochorismatase hydrolase family low similarity to SP |
| At5g56040 | leucine rich repeat protein kinase, putative contains leucine rich repeat (LRR) |
| | domains, Pfam: PF00560; contains protein kinase domain, Pfam: PF00069 |
| At5g22870 | hypothetical protein similar to unknown protein (gb |
| At3g23955 | pseudogene, similar to hypothetical protein GB: AAD29066 |
| At1g31090 | hypothetical protein contains similarity to gi |
| At1g14250 | hypothetical protein |
| At1g74190 | leucine rich repeat protein family contains leucine rich-repeat (LRR) domains |
| | Pfam: PF00560, INTERPRO: IPR001611; contains similarity to Cf-2.1 |
| | [Lycopersicon pimpinellifolium] gi |
| At1g51160 | expressed protein |
| At3g26310 | cytochrome P450 family contains Pfam profile: PF00067 cytochrome P450 |
| At1g18750 | MADS-box protein similar to homeodomain transcription factor (AGL30) |
| | GI: 3461830 from [Arabidopsis thaliana] |
| At5g17820 | peroxidase, putative identical to peroxidase ATP13a [Arabidopsis thaliana] gi |
| At2g39880 | myb family transcription factor (MYB25) contains Pfam profile: PF00249 myb-like |
| | DNA-binding domain |
| At2g20240 | expressed protein |
| At2g44210 | expressed protein Pfam profile PF03080: Arabidopsis proteins of unknown |
| | function |
| At3g12970 | expressed protein |
| At3g09030 | expressed protein identical to GB: AAD56319 [Arabidopsis thaliana] |
| At3g02250 | auxin-independent growth promoter-related similar to auxin-independent growth |
| | promoter GB: A44226 [Nicotiana tabacum] |
| At4g23380 | hypothetical protein predicted proteins, Arabidopsis thaliana |
| At4g23110 | hypothetical protein |
| At4g13990 | hypothetical protein |
| At5g43670 | protein transport protein SEC23 |
| At5g59800 | hypothetical protein |
| At5g16530 | auxin efflux carrier protein family contains auxin efflux carrier domain, |
| | Pfam: PF03547 |
| At4g35410 | clathrin assembly protein AP19 homolog |
| At2g15000 | expressed protein |
| At3g60830 | actin - like protein actin 3, Drosophila melanogaster, PIR: A03000 |
| At2g21770 | cellulose synthase, catalytic subunit, putative similar to gi: 2827141 cellulose |
| | synthase catalytic subunit, Arabidopsis thaliana (Ath-A) |
| At1g09240 | nicotianamine synthase, putative similar to nicotianamine synthase |
| | [Lycopersicon esculentum][GI: 4753801], nicotianamine synthase 2 [Hordeum |
| | vulgare][GI: 4894912] |
| At5g26780 | glycine hydroxymethyltransferase - like protein glycine |
| | hydroxymethyltransferase, Solanum tuberosum, EMBL: Z25863 |
| At1g22910 | RNA recognition motif (RRM) - containing protein contains InterPro entry |
| | IPR000504: RNA-binding region RNP-1 (RNA recognition motif) (RRM); similar |
| | to GB: AAC33496 |
| At1g42460 | Ulp1 protease family contains Pfam profile PF02902: Ulp1 protease family, C- |
| | terminal catalytic domain |
| At3g09670 | PWWP domain protein |
| At3g44200 | protein kinase family contains protein kinase domain, Pfam: PF00069; contains |
| | serine/threonine protein kinase domain, INTERPRO: IPR002290 |
| At4g09350 | DnaJ protein family similar to SP |
| At5g39880 | expressed protein |
| At5g67490 | expressed protein |
| At5g58860 | cytochrome P450 86A1 identical to Cytochrome P450 86A1 (CYPLXXXVI) |
| | (P450-dependent fatty acid omega-hydroxylase) (SP: P48422) [Arabidopsis |
| | thaliana] |
| At5g16070 | chaperonin, putative similar to SWISS-PROT: P80317 T-complex protein 1, zeta |
| | subunit (TCP-1-zeta) [Mus musculus]; contains Pfam: PF00118 domain, TCP- |
| | 1/cpn60 chaperonin family |
| At3g52680 | F-box protein family contains F-box domain Pfam: PF00646 |
| At2g35170 | expressed protein |
| At2g23300 | leucine-rich repeat transmembrane protein kinase, putative |
| At5g36070 | hypothetical protein strong similarity to unknown protein (emb |
| At5g49780 | leucine-rich repeat transmembrane protein kinase, putative |
| At1g37150 | biotin holocarboxylase synthetase-related similar to biotin holocarboxylase |
| | synthetase GI: 4874309 from [Arabidopsis thaliana] contains non-consensus GG |
| | acceptor splice sites. |
| At1g79710 | hypothetical protein similar to hypothetical protein GB: AAC12874 |
| | [Synechococcus PCC7942] |
| At1g16420 | hypothetical protein common family similar to At5g04200, At1g79340, |
| | At1g79320, At1g79310, At1g79330; similar to latex-abundant protein |
| | [GI: 4235430][Hevea brasiliensis] |
| At2g33310 | auxin-responsive protein IAA13 (Indoleacetic acid-induced protein 13) identical |
| | to SP |
| At2g22100 | RRM-containing RNA-binding protein |
| At3g62680 | proline-rich protein family contains proline-rich region, INTERPRO: IPR000694 |
| At3g22180 | DHHC-type zinc finger domain-containing protein contains Pfam profile |
| | PF01529: DHHC zinc finger domain |
| At3g21465 | adenyl cyclase-related similar to adenyl cyclase GB: AAB87670 from [Nicotiana |
| | tabacum] |
| At4g16420 | transcriptional adaptor like protein |
| At1g47705 | pseudogene, putative peroxidase similar to peroxidase GB: P00434 GI: 464365 |
| | from [Brassica rapa] |
| At1g10870 | ARF GTPase-activating domain-containing protein |
| At1g07250 | glycosyltransferase family similar to UDP-glucose glucosyltransferase GI: 453245 |
| | from [Manihot esculenta] |
| At1g64105 | No apical meristem (NAM) protein family contains Pfam PF02365: No apical |
| | meristem (NAM) domain |
| At1g10660 | expressed protein |
| At1g60800 | receptor-related kinase similar to somatic embryogenesis receptor-like kinase |
| | GI: 2224910 from [Daucus carota] |
| At2g32140 | disease resistance protein (TIR class), putative domain signature TIR exists, |
| | suggestive of a disease resistance protein. |
| At2g33090 | hypothetical protein |
| At2g47280 | pectinesterase family contains Pfam profile: PF01095 pectinesterase |
| At2g41020 | expressed protein |
| At2g44310 | calcium-binding EF-hand family protein contains INTERPRO: IPR002048 |
| | calcium-binding EF-hand domain |
| At2g41450 | GCN5-related N-acetyltransferase (GNAT) family low similarity to Swift [Xenopus |
| | laevis] GI: 14164561; contains Pfam profiles PF00583: acetyltransferase, GNAT |
| | family, PF00533: BRCA1 C Terminus (BRCT) domain |
| At3g23175 | expressed protein supported by RACE-based full-length cDNA validates this |
| | gene structure. (Brassica genome sequence alignment supported. Work by |
| | cdtown, et al.) |
| At3g20840 | ovule development protein, putative similar to ovule development protein |
| | AINTEGUMENTA (GI: 1209099) [Arabidopsis thaliana] |
| At4g36750 | quinone reductase family similar to 1,4-benzoquinone reductase [Phanerochaete |
| | chrysosporium][GI: 4454993]; similar to Trp repressor binding protein |
| | [Escherichia coli][SP |
| At4g29000 | transcription factor-related leghemoglobin activating factor - Glycine max, |
| | PID: e1374538 |
| At5g22420 | acyl CoA reductase-related protein |
| At5g01370 | hypothetical protein |
| At5g03960 | calmodulin-binding protein - related |
| At5g52050 | MATE efflux protein - related contains Pfam profile PF01554: Uncharacterized |
| | membrane protein family |
| At4g29430 | 40S ribosomal protein S15A (RPS15aE) ribosomal protein S15a - Brassica |
| | napus, PIR2: S20945 |
| At2g12700 | hypothetical protein similar to GB: AAD23022 |
| At4g05520 | calcium-binding EF-hand family protein similar to EH-domain containing protein |
| | 1 from {Mus musculus} SP |
| At3g24020 | disease resistance response protein-related contains similarity to disease |
| | resistance response protein 206-d [Pisum sativum] gi |
| At5g63690 | hypothetical protein |
| At3g46670 | glucosyltransferase-related protein UDP-glucose glucosyltransferase - |
| | Arabidopsis thaliana, EMBL: AB016819 |
| At4g18010 | inositol polyphosphate 5-phosphatase II (IP5PII) nearly identical to inositol |
| | polyphosphate 5-phosphatase II [Arabidopsis thaliana] GI: 10444263 isoform |
| | contains an AT-acceptor splice site at intron 6 |
| At1g80480 | expressed protein contains Viral RNA helicase domain |

TABLE III

| TAIR | |
|---|---|
| accession | |
| No. | Description (homologous genes identified in other organisms) |
| At2g40100 | light-harvesting chlorophyll a/b binding protein |
| At5g03880 | auxin-regulated protein predicted protein, Arabidopsis thaliana |
| At4g10510 | subtilisin-like serine protease contains similarity to subtilase; SP1 GI: 9957714 |
| | from [Oryza sativa] |
| At2g33840 | tyrosyl-tRNA synthetase-related |
| At5g05670 | signal recognition particle receptor beta subunit-related protein |
| At1g53290 | galactosyltransferase family contains Pfam profile: PF01762 |
| | galactosyltransferase; contains similarity to Avr9 elicitor response protein |
| | GI: 4138265 from [Nicotiana tabacum] |
| At1g32700 | expressed protein similar to hypothetical protein GB: AAF25968 GI: 6714272 from |
| | [Arabidopsis thaliana] |
| At5g16770 | myb DNA-binding protein (AtMYB9) |
| At3g59200 | F-box protein family contains F-box domain Pfam: PF00646 |
| At3g43380 | hypothetical protein hypothetical proteins - Arabidopsis thaliana |
| At5g38120 | 4-coumarate:CoA ligase (4-coumaroyl-CoA synthase) family similar to 4CL2, |
| | Arabidopsis thaliana [gi: 12229665], 4CL1, Nicotiana tabacum [gi: 12229631]; |
| | contains Pfam AMP-binding enzyme domain PF00501 |
| At4g07750 | transposon protein-related similar to Arabidopsis thaliana putative En/Spm |
| | transposon protein (GB: AC005396) |
| At4g04710 | calcium-dependent protein kinase, putative (CDPK) similar to calcium-dependent |
| | protein kinase [Nicotiana tabacum] gi |
| At5g13350 | auxin-responsive - like protein Nt-gh3 deduced protein, Nicotiana tabacum, |
| | EMBL: AF123503 |
| At2g20410 | expressed protein |
| At5g39730 | avirulence induced gene (AIG) - like protein AIG2 PROTEIN, Arabidopsis |
| | thaliana, SWISSPROT: AIG2_ARATH |
| At4g29905 | expressed protein |
| At3g07300 | expressed protein similar to translation initiation factor EIF-2B beta subunit |
| | GB: Q90511 [Fugu rubripes] |
| At4g30230 | hypothetical protein |
| At2g01710 | DnaJ protein family simlar to AHM1 [Triticum aestivum] GI: 6691467; contains |
| | Pfam profile PF00226: DnaJ domain |
| At2g34780 | hypothetical protein |
| At4g20960 | expressed protein riboflavin biosynthesis protein ribG, Synechocystissp., |
| | PIR2: S74377 |
| At2g19750 | 40S ribosomal protein S30 (RPS30A) |
| At5g65850 | F-box protein family |
| At5g45830 | tumor-related protein-like |
| At2g23070 | casein kinase II alpha chain, putative similar to casein kinase II, alpha chain (CK |
| | II) [Zea mays] SWISS-PROT: P28523; contains protein kinase domain, |
| | Pfam: PF00069 |
| At1g05300 | metal transporter, putative (ZIP5) identical to putative metal transporter ZIP5 |
| | [Arabidopsis thaliana] gi |
| At1g36180 | acetyl-CoA carboxylase-related similar to GI: 1100253 from [Arabidopsis |
| | thaliana] |
| At4g12390 | pectinesterase-related low similarity to pectinesterase from Arabidopsis |
| | thaliana SP |
| At3g52520 | hypothetical protein |
| At5g24600 | expressed protein similar to unknown protein (pir |
| At1g78890 | expressed protein |
| At5g56670 | 40S ribosomal protein S30 (RPS30C) |
| At4g00730 | homeodomain protein AHDP |
| At2g44630 | Kelch repeat containing F-box protein family similar to SKP1 interacting partner |
| | 6 [Arabidopsis thaliana] GI: 10716957; contains Pfam profiles PF00646: F-box |
| | domain, PF01344: Kelch motif |
| At2g10850 | envelope-related protein identical to GB: AAD20656 |
| At4g39360 | hypothetical protein |
| At4g23030 | MATE efflux protein-related contains Pfam profile PF01554: Uncharacterized |
| | membrane protein family |
| At1g01320 | tetratricopeptide repeat (TPR)-containing protein low similarity to SP |
| At3g32400 | proline-rich protein family common family members: At2g43800, At3g25500, |
| | At5g48360, At4g15200, At3g05470, At3g07540, At5g07780, At5g07650 |
| | [Arabidopsis thaliana]; |
| At1g17230 | leucine rich repeat protein family contains protein kinase domain, |
| | Pfam: PF00069; contains leucine-rich repeats, Pfam: PF00560 |
| At3g29612 | pseudogene, hypothetical protein |
| At4g27280 | calcium-binding EF-hand family protein similar to EF-hand Ca2+-binding protein |
| | CCD1 [Triticum aestivum] GI: 9255753; contains INTERPRO: IPR002048 |
| | calcium-binding EF-hand domain |
| At5g54920 | expressed protein strong similarity to unknown protein (pir |
| At3g01810 | expressed protein similar to unknown protein |
| At4g03140 | short-chain dehydrogenase/reductase family protein similar to stem |
| | secoisolariciresinol dehydrogenase GI: 13752458 from {Forsythia x intermedia}; |
| | similar to sex determination protein tasselseed 2 SP: P50160 from [Zea mays] |
| At4g16920 | disease resistance protein (TIR-NBS-LRR class), putative domain signature TIR- |
| | NBS-LRR exists, suggestive of a disease resistance protein. |
| At5g47790 | expressed protein |
| At3g06920 | pentatricopeptide (PPR) repeat-containing protein low similarity to fertility |
| | restorer [Petunia x hybrida] GI: 22128587; contains Pfam profile PF01535: PPR |
| | repeat |
| At2g21790 | ribonucleoside-diphosphate reductase large subunit-related |
| At5g02620 | ankyrin repeat protein family contains ankyrin repeat domains, Pfam: PF00023 |
| At1g26930 | Kelch repeat containing F-box protein family contains Pfam: PF01344 Kelch |
| | motif, Pfam: PF00646 F-box domain |
| At5g47200 | GTP-binding protein, putative similar to GTP-binding protein GI: 303750 from |
| | [Pisum sativum] |
| At5g57180 | CIA2 (CIA2) annotation temporarily based on supporting cDNA gi |
| At5g66230 | expressed protein similar to unknown protein (emb |
| At2g01770 | membrane protein-related |
| At5g08100 | Asparaginase |
| At1g52940 | calcineurin-like phosphoesterase family contains Pfam profile: PF00149 |
| | calcineurin-like phosphoesterase |
| At3g61640 | arabinogalactan-protein (AGP20) |
| At4g04870 | CDP-alcohol phosphatidyltransferase family similar to SP |
| At3g02370 | hypothetical protein |
| At4g20790 | leucine rich repeat protein family contains leucine rich repeat (LRR) domains, |
| | Pfam: PF00560; |
| At3g03480 | transferase family similar to hypersensitivity-related gene GB: CAA64636 |
| | [Nicotiana tabacum]; contains Pfam transferase family domain PF00248 |
| At1g71030 | myb family transcription factor similar to MybHv5 GI: 19055 from [Hordeum |
| | vulgare] |
| At1g02410 | expressed protein contains similarity to cytochrome c oxidase assembly protein |
| | cox11 GI: 1244782 from [Saccharomyces cerevisiae] |
| At5g64550 | expressed protein strong similarity to unknown protein (emb |
| At4g30370 | C3HC4-type zinc finger protein family contains Pfam profile: PF00097 zinc |
| | finger, C3HC4 type (RING finger) |
| At1g12100 | protease inhibitor/seed storage/lipid transfer protein (LTP) family contains Pfam |
| | protease inhibitor/seed storage/LTP family domain PF00234 |
| At4g36210 | expressed protein F35D11.3, Caenorhabditis elegans, PATX: G868225 |
| At2g24180 | cytochrome P450 family |
| At5g56410 | F-box protein family contains F-box domain Pfam: PF00646 |
| At4g15830 | expressed protein |
| At1g62170 | serpin family similar to phloem serpin-1 GI: 9937311 from [Cucurbita maxima]; |
| | contains Pfam profile PF00079: Serpin (serine protease inhibitor) |
| At4g20000 | expressed protein |
| At3g13290 | transducin/WD-40 repeat protein family contains 2 WD-40 repeats (PF00400); |
| | autoantigen locus HUMAUTANT (GI: 533202) [Homo sapiens] and autoantigen |
| | locus HSU17474 (GI: 596134) [Homo sapiens] |
| At1gp7620 | GTP-binding protein-related similar to GB: M24537 from [Bacillus subtilis] |
| At4g24690 | ubiquitin-associated (UBA)/PB1 domain-containing protein contains Pfam |
| | profiles PF00627: Ubiquitin-associated (UBA)/TS-N domain, PF00569: Zinc |
| | finger ZZ type domain, PF00564: PB1 domain |
| At5g04750 | F1F0-ATPase inhibitor - like protein F1F0-ATPase inhibitor protein, OsIF1-1, |
| | Oryza sativa, EMBL: AB029059 |
| At3g26750 | hypothetical protein |
| At5g52610 | F-box protein family contains F-box domain Pfam: PF00646 |
| At1g20190 | expansin, putative (EXP11) similar to GB: U30460 from [Cucumis sativus]; alpha- |
| | expansin gene family, PMID: 11641069 |
| At3g01310 | expressed protein similar to unknown protein GB: BAA24863 [Homo sapiens], |
| | unknown protein GB: BAA20831 [Homo sapiens], unknown protein |
| | GB: AAB42264 [Caenorhabditis elegans] |
| At5g46180 | ornithine--oxo-acid aminotransferase (ornithine aminotransferase/ornithine |
| | ketoacid aminotransferase), putative similar to SP |
| At5g47250 | disease resistance protein (CC-NBS-LRR class), putative domain signature CC- |
| | NBS-LRR exists, suggestive of a disease resistance protein. |
| At3g46480 | oxidoreductase, 2OG-Fe(II) oxygenase family low similarity to gibberellin 20- |
| | oxidase [gi: 4678370]; contains Pfam domain PF03171, 2OG-Fe(II) oxygenase |
| | superfamily |
| At2g28290 | SNF2 domain/helicase domain-containing protein similar to transcriptional |
| | activator HBRM [Homo sapiens] GI: 414117; contains Pfam profiles PF00271: |
| | Helicase conserved C-terminal domain, PF00176: SNF2 family N-terminal |
| | domain |
| At3g55660 | hypothetical protein various predicted proteins, Arabidopsis thaliana |
| At1g71740 | hypothetical protein |
| At1g17940 | hypothetical protein |
| At5g51440 | heat shock protein, putative similar to heat shock 22 kDa protein, mitochondrial |
| | precursor SP: Q96331 from [Arabidopsis thaliana] |
| At1g10660 | expressed protein |
| At1g23720 | proline-rich protein family contains proline-rich extensin domains, |
| | INTERPRO: IPR002965 |
| At3g12700 | expressed protein |
| At2g31380 | salt tolerance-like protein |
| At5g49160 | DNA (cytosine-5)-methyltransferase (DNA methyltransferase) (DNA metase) (sp |
| At4g13900 | disease resistance protein family contains leucine rich-repeat domains |
| | Pfam: PF00560, INTERPRO: IPR001611; similar to Cf-4A protein [Lycopersicon |
| | esculentum] gi |
| At5g56160 | sec14 cytosolic factor family (phosphoglyceride transfer protein family) similar to |
| | SFC14 cytosolic factor (SP: P45816) [Candida lipolytica] |
| At5g05780 | 26S proteasome regulatory subunit S12 (RPN8), putative contains similarity to |
| | 26s proteasome regulatory subunit s12 (proteasome subunit p40) (mov34 |
| | protein) SP: P26516 from [Mus musculus] |
| At4g29410 | 60S ribosomal protein L28 (RPL28C) unknown protein chromosome II BAC |
| | F6F22 - Arabidopsis thaliana, PID: g3687251 |
| At3g07440 | expressed protein est hits to genscan model |
| At1g79350 | expressed protein |
| At5g35180 | expressed protein |
| At5g08520 | expressed protein contains similarity to I-box binding factor |
| At1g03680 | thioredoxin M-type 1, chloroplast precursor (TRX-M1) nearly identical to SP |
| At4g07340 | contains similarity to Xenopus laevis replication protein A1 (SW: RFA1_XENLA) |
| At3g50440 | hydrolase, alpha/beta fold family similar to ethylene-induced esterase [Citrus |
| | sinensis] GI: 14279437, polyneuridine aldehyde esterase [Rauvolfia serpentina] |
| | GI: 6651393; contains Pfam profile PF00561: hydrolase, alpha/beta fold family |
| At1g54480 | leucine rich repeat protein family contains leucine rich-repeat (LRR) domains |
| | Pfam: PF00560, INTERPRO: IPR001611; contains similarity to disease |
| | resistance protein GI: 3894383 from [Lycopersicon esculentum] |
| At3g13222 | expressed protein |
| At4g34570 | bifunctional dihydrofolate reductase-thymidylate synthase 2 (DHFR-TS) (THY-2) |
| | identical to SP |

TABLE IV

| TAIR | |
|---|---|
| accession | |
| No. | Description (homologous genes identified in other organisms) |
| At3g27690 | light harvesting chlorophyll A/B binding protein, putative similar to chlorophyll A- |
| | B binding protein 151 precursor (LHCP) GB: P27518 from [Gossypium |
| | hirsutum] |
| At3g48730 | glutamate-1-semialdehyde 2,1-aminomutase 2 (GSA 2) (glutamate-1- |
| | semialdehyde aminotransferase 2) (GSA-AT 2) identical to GSA2 [SP |
| At3g18810 | protein kinase-related similar to somatic embryogenesis receptor-like kinase |
| | GB: AAB61708 from [Daucus carota] |
| At3g58450 | expressed protein ethylene-responsive protein ER6 - Lycopersicon esculentum, |
| | EMBL: AF096262 |
| At2g18040 | peptidyl-prolyl cis-trans isomerase-related similar to ESS1 (S. cerevisiae) and |
| | dodo (D. melanogaster.) |
| At3g03800 | syntaxin of plants SYP131 similar to s-syntaxin GB: CAA74913 [Loligo pealei] |
| At4g38740 | peptidylprolyl isomerase ROC1 |
| At5g15490 | UDP-glucose dehydrogenase-related protein UDP-glucose 6-dehydrogenase - |
| | Glycine max, EMBL: U53418 |
| At1g74150 | Kelch repeat-containing protein low similarity to rngB protein, Dictyostelium |
| | discoideum, PIR: S68824; contains Pfam profile PF01344: Kelch motif |
| At5g40520 | expressed protein predicted proteins, Arabidopsis thaliana |
| At4g10020 | short-chain dehydrogenase/reductase family protein similar to sterol-binding |
| | dehydrogenase steroleosin GI: 15824408 from [Sesamum indicum] |
| At2g14630 | hypothetical protein contains Pfam profile PF03004: Plant transposase |
| | (Ptta/En/Spm family) |
| At3g08943 | pseudogene, importin beta subunit similar to importin-beta1 GB: BAA34861, |
| | importin-beta2 GB: BAA34862 [Oryza sativa]; frameshift |
| At2g41970 | protein kinase, putative similar to Pto kinase interactor 1 (serine/threonine |
| | protein kinase) [Lycopersicon esculentum] gi |
| At2g04750 | fimbrin-related |
| AtCg00040 | matK: maturase |
| At5g06340 | diadenosine 5′,5′″-P1,P4-tetraphosphate hydrolase, putative similar to |
| | diadenosine 5′,5′″-P1,P4-tetraphosphate hydrolase from [Lupinus angustifolius] |
| | GI: 1888557, [Hordeum vulgare subsp. vulgare] GI: 2564253; contains Pfam |
| | profile PF00293: NUDIX domai? |
| At1g17170 | glutathione transferase, putative One of three repeated putative glutathione |
| | transferases. 72% identical to glutathione transferase [Arabidopsis thaliana] (gi |
| AtCg00960 | rrn4.5S:23S ribosomal RNA |
| At1g67150 | hypothetical protein |
| At2g24400 | auxin-induced (indole-3-acetic acid induced) protein, putative (SAUR_d) similar |
| | to SAUR-AC-like protein (small auxin up RNA) (GI: 4455308) from [Arabidopsis |
| | thaliana]; auxin-induced protein TGSAUR22 (GI: 10185820) [Tulipa gesnerian] |
| At1g06560 | expressed protein |
| At2g43640 | signal recognition particle protein 14 kD, ATSRP14-related |
| At5g61700 | ABC transporter family protein ABC family transporter, Entamoeba histolytica, |
| | EMBL: EH058 |
| AtCg01050 | ndhD: NADH dehydrogenase subunit 4 |
| At1g78980 | leucine-rich repeat transmembrane protein kinase, putative similar to leucine- |
| | rich repeat transmembrane protein kinase 2 GI: 3360291 from [Zea mays] |
| At2g24640 | ubiquitin carboxyl terminal hydrolase-related |
| At3g50170 | hypothetical protein various predicted genes, Arabidopsis thaliana and Oryza |
| | sativa |
| At3g25190 | integral membrane protein-related contains Pfam profile: PF01988 integral |
| | membrane protein; similar to nodulin-21 GB: CAA34506 [Glycine max] |
| At5g41970 | GAMM1 protein-related |
| At5g58020 | expressed protein protein × 0001, Homo sapiens, EMBL: AF117231 |
| At1g52080 | expressed protein |
| At5g55050 | GDSL-motif lipase/hydrolase protein similar to family II lipases EXL3 |
| | GI: 15054386, EXL1 GI: 15054382, EXL2 GI: 15054384 from [Arabidopsis |
| | thaliana]; contains Pfam profile PF00657: GDSL-like Lipase/Acylhydrolase |
| At4g30610 | serine carboxypeptidase-related probable SERINE CARBOXYPEPTIDASE II-2 |
| | PRECURSOR - HORDEUM VULGARE, PIR2: T05701 |
| At1g19210 | AP2 domain transcription factor, putative similar to AP2 domain transcription |
| | factor GI: 4567204 from [Arabidopsis thaliana] |
| At2g33330 | expressed protein contains Pfam PF01657: Domain of unknown function |
| At1g13940 | expressed protein identical to hypothetical protein GB: AAD39280 GI: 5080770 |
| | from [Arabidopsis thaliana] |
| At5g09840 | expressed protein similar to unknown protein (emb |
| At1g11915 | expressed protein |
| At2g41620 | expressed protein |
| At5g56910 | expressed protein similar to unknown protein (pir |
| At1g62200 | oligopeptide transporter-related similar to oligopeptide transporter 1-1 |
| | GI: 510238 from [Arabidopsis thaliana]; contains non-consensus GA donor site |
| | at intron 4 |
| At2g18640 | geranylgeranyl pyrophosphate synthase |
| | (GGPS2/GGPS5)(farnesyltranstransferase), putative similar to gi: 1944371; |
| | contains GB: L22347 |
| At4g09260 | hypothetical protein nearly identical with protein T8A17_40 cause of location on |
| | repetitive section |
| At5g14240 | expressed protein various predicted proteins from D. melanogaster, H. sapiens |
| | and S. pombe |
| At5g17310 | UDP-glucose pyrophosphorylase |

Preferably, said targeting nucleic acid has a transcribed sequence which is that of an mRNA of a gene selected from the group consisting of the genes shown in Table V. The genes shown in Table V correspond to the gene encoding the eukaryotic translation initiation factor eIF4E and to a selection of genes appearing in Table III, which have an expression differential in favor of stroma versus chloroplast, in favor of stroma versus total RNA, and in favor of chloroplast versus total RNA.

TABLE V

| TAIR | | |
|---|---|---|
| accession | | Coding |
| No. | Description | sequence |
| At5g38120 | 4-coumarate:CoA ligase (4-coumaroyl-CoA synthase) family | SEQ ID No. 1 |
| | similar to 4CL2, Arabidopsis thaliana [gi: 12229665], 4CL1, | |
| | Nicotiana tabacum [gi: 12229631]; contains Pfam AMP-binding | |
| | enzyme domain PF00501 | |
| At4g04710 | calcium-dependent protein kinase, putative (CDPK) similar to | SEQ ID No. 3 |
| | calcium-dependent protein kinase [Nicotiana tabacum] gi | |
| At5g13350 | auxin-responsive - like protein Nt-gh3 deduced protein, | SEQ ID No. 5 |
| | Nicotiana tabacum, EMBL: AF123503 | |
| At4g30230 | hypothetical protein | SEQ ID No. 7 |
| At2g01710 | DnaJ protein family simlar to AHM1 [Triticum aestivum] | SEQ ID No. 9 |
| | GI: 6691467; contains Pfam profile PF00226: DnaJ domain | |
| At2g19750 | 40S ribosomal protein S30 (RPS30A) | SEQ ID No. 11 |
| At5g24600 | expressed protein similar to unknown protein (pir | SEQ ID No. 13 |
| At5g56670 | 40S ribosomal protein S30 (RPS30C) | SEQ ID No. 15 |
| At2g44630 | Kelch repeat containing F-box protein family similar to SKP1 | SEQ ID No. 17 |
| | interacting partner 6 [Arabidopsis thaliana] GI: 10716957; | |
| | contains Pfam profiles PF00646: F-box domain, PF01344: | |
| | Kelch motif | |
| At2g10850 | envelope-related protein identical to GB: AAD20656 | SEQ ID No. 19 |
| At3g01810 | expressed protein similar to unknown protein | SEQ ID No. 21 |
| At5g47200 | GTP-binding protein, putative similar to GTP-binding protein | SEQ ID No. 23 |
| | GI: 303750 from [Pisum sativum] | |
| At5g66230 | expressed protein similar to unknown protein (emb | SEQ ID No. 25 |
| At1g71030 | myb family transcription factor similar to MybHv5 GI: 19055 from | SEQ ID No. 27 |
| | [Hordeum vulgare] | |
| At1g02410 | expressed protein contains similarity to cytochrome c oxidase | SEQ ID No. 29 |
| | assembly protein cox11 GI: 1244782 from [Saccharomyces | |
| | cerevisiae] | |
| At5g52610 | F-box protein family contains F-box domain Pfam: PF00646 | SEQ ID No. 31 |
| At2g28290 | SNF2 domain/helicase domain-containing protein similar to | SEQ ID No. 33 |
| | transcriptional activator HBRM [Homo sapiens] GI: 414117; | |
| | contains Pfam profiles PF00271: Helicase conserved C- | |
| | terminal domain, PF00176: SNF2 family N-terminal domain | |
| At1g10660 | expressed protein | SEQ ID No. 35 |
| At5g49160 | DNA (cytosine-5)-methyltransferase (DNA methyltransferase) | SEQ ID No. 37 |
| | (DNA metase) (sp | |
| At5g35180 | expressed protein | SEQ ID No. 39 |
| At4g18040 | translation initiation factor eIF4E | SEQ ID No. 41 |

According to a preferred embodiment, said targeting nucleic acid has a transcribed sequence which is that of an mRNA of the gene encoding the eukaryotic translation initiation factor eIF4E (SEQ ID No. 41) and/or its homologues in the other species, and more particularly in tomato and capsicum (patent WO 03/066900).

The transcribed sequence of the targeting nucleic acid used to implement the method according to the invention is preferably that of an mRNA of a nuclear gene which is endogenous (i.e. naturally present in the nuclear genome of the cell which is transformed) or which is exogenous to the plant cell (for example, a nuclear gene for which an mRNA has been detected in a plastid in a cell belonging to a plant species other than that of the transformed cell). Preferably, the nuclear gene is endogenous to the transformed plant cell.

The expression “nucleic acid of interest linked to a targeting nucleic acid” is intended to mean that the nucleic acid of interest and the targeting nucleic acid are genetically linked, i.e. they are part of the same nucleotide construct (DNA, RNA or mixed DNA/RNA). Preferably, said nucleic acid of interest is fused to said targeting nucleic acid.

The nucleic acid of interest can be a DNA sequence or an RNA sequence. Similarly, the targeting nucleic acid can be a DNA sequence or an RNA sequence. The nucleic acid of interest linked to the targeting nucleic acid can be a mixed DNA/RNA nucleic acid. Preferably, the nucleic acid of interest and the targeting nucleic acid are both a DNA sequence. Also preferably, the nucleic acid of interest and the targeting nucleic acid are both an RNA sequence.

The plant cell can be transformed with a nucleic acid of interest linked to a targeting nucleic acid in such a way as to obtain stable expression of at least said nucleic acid of interest. According to this embodiment, the nucleic acid of interest linked to a targeting nucleic acid can be in the form of a construct comprising a DNA sequence of interest linked to a targeting DNA sequence, said construct being integrated into the nuclear genome of the plant cell. The transcription of this construct in the nucleus produces a transcript comprising an RNA sequence of interest linked to a targeting RNA sequence, the transcript then being targeted to a plastid of the transformed cell.

The plant cell can also be transformed with a nucleic acid of interest linked to a targeting nucleic acid in such a way as to obtain transient expression of at least said nucleic acid of interest, by methods known to those skilled in the art such as, for example, the use of polyethylene glycol (PEG), which is a nontoxic molecule capable of inducing destabilization of the plasma membrane and which allows DNA to be transferred through said membrane. The DNA molecules can then migrate to the nucleus, where some of them, with less or greater effectiveness, are capable of integrating into the chromosomes. The DNA can also be encapsulated in liposomes, which are small artificial vesicles of phospholipids, capable of fusing with protoplasts. Finally, it is possible to perform electroporation of protoplasts, which consists in subjecting a mixture of protoplasts and DNA to a series of short-duration, high-voltage electric shocks. These methods make it possible to study transient expression in protoplasts, and to obtain transgenic plants for species in which regeneration from protoplasts can be successfully performed. The nucleic acid of interest linked to a targeting nucleic acid can therefore be in the form of a construct comprising an RNA sequence of interest linked to a targeting RNA sequence, said construct being integrated into the cytosol of the plant cell. This RNA construct is then targeted to a plastid of the transformed cell.

Said nucleic acid of interest can be a sequence encoding a protein, in particular a heterologous protein. The term “heterologous protein” is intended to mean a protein which is not expressed by the nontransformed plant cell. It may be a recombinant protein normally expressed in a eukaryotic organism, for example a protein of human, animal or plant origin. It may in particular be:
a protein of therapeutic and/or prophylactic interest, such as insulin, gastric lipase, collagen or an allergen;
the HppD or 4-hydroxyphenylpyruvate dioxygenase protein of Pseudomonas fluorescens, which makes it possible to modulate the biosynthesis of tocopherol in plants (HPPD; Garcia et al. 1997,1999; Norris et al., 1998);
a protein which confers resistance to a herbicide, such as the precursor of acetolactate synthetase (ALS) (Lee et al., 1988), mutated acetolactate synthetase (Preston and Powles, 2002), or 3-enolpyruvylshikimate-5-phosphate synthetase (EPSP synthetase) (Klee et al., 1987);
a protein which confers a capacity for fixing nitrogen or for increased photosynthesis, a protein which confers increased resistance to drought, to salt, or to extreme temperatures, for example;
a protein which confers resistance to pathogens such as insects, fungi, bacteria, viruses, etc, such as a protease inhibitor, for example a trypsin inhibitor (Hilder et al., 1987), a toxin, for example the toxins of Bacillus thuringiensis (Vaeck et al., 1987; Fischhoff et al., 1987), etc.

Said nucleic acid of interest can also be a noncoding sequence, such as an antisense RNA sequence or a DNA sequence of which the transcript is an antisense RNA, or else an interfering RNA (iRNA, Sharp, 2001). For a general description of antisense technology, those skilled in the art can refer, for example, to the book “Antisense DNA and RNA” (Cold Spring Harbor Laboratory, D. Melton, ed. 1988).

The nucleotide construct comprising the nucleic acid of interest and the targeting nucleic acid can be prepared by any method known to those skilled in the art, for example by in vitro synthesis.

The nucleotide construct used to transform the plant cell in the targeting method according to the invention is also a subject of the present application. The invention relates more particularly to a nucleic acid construct comprising a nucleic acid of interest linked to a targeting nucleic acid, the transcribed sequence of which is that of an mRNA of a nuclear gene selected from the group consisting of the genes represented in Table V. Said nuclear gene is therefore selected from the group of genes having, as coding sequence, SEQ ID No.1, SEQ ID No.3, SEQ ID No.5, SEQ ID No.7, SEQ ID No.9, SEQ ID No.11, SEQ ID No.13, SEQ ID No.15, SEQ ID No.17, SEQ ID No.19, SEQ ID No.21, SEQ ID No.23, SEQ ID No.25, SEQ ID No.27, SEQ ID No.29, SEQ ID No.31, SEQ ID No.33, SEQ ID No.35, SEQ ID No.37, SEQ ID No.39 and SEQ ID No.41 identified in Arabidopsis thaliana and/or their homologous sequences in other species.

The nucleotide constructs used in the invention can be expression cassettes comprising a nucleic acid sequence of interest linked to a targeting nucleic acid sequence, combined with elements for the expression of the nucleic acid sequence of interest in plant cells, in particular a transcription promoter and a transcription terminator, or else an activator. Other elements, such as introns, enhancers, polyadenylation sequences and derivatives, can also be present. The expression cassette can also contain 5′ untranslated sequences, referred to as “leader” sequences. Such sequences can enhance translation.

A very large number of transcription promoters can be used for the expression in plant cells. This may involve a constitutive promoter, such as the actin-intron-actin promoter, corresponding to the 5′ noncoding region of the rice actin 1 gene and its first intron (McElroy et al., 1991; GenBank No. S44221). The presence of the first actin intron makes it possible to increase the level of expression of a gene when it is fused in the position 3′ of a promoter. It may also involve an inducible or tissue-specific promoter, for example so that the nucleic acid of interest is targeted to a plastid only at certain developmental stages of the plant, only under certain environmental conditions, or only in certain target tissues. Examples of tissue-specific promoters include the Chlorelle virus promoter which regulates the expression of the adenine methyl transferase gene (Mitra and Higgins, 1994) or the cassaya mosaic virus promoter (Verdaguer et al., 1998) which is expressed mainly in green tissues, or the regulatory elements of the tomato 2A11 gene promoter which allow specific expression in the fruits (Van Haaren and Houck, 1991).

Among the terminators, mention may in particular be made of:
the 3′ Nos. terminator, nopaline synthase terminator, which corresponds to the 3′ noncoding region of the nopaline synthase gene originating from the Ti plasmid of Agrobacterium tumefaciens nopaline strain (Depicker et al., 1982), and
the 3′ CaMV terminator, corresponding to the 3′ noncoding region of the cauliflower mosaic circular double-stranded DNA virus sequence which produces the 35S transcript (Franck et al. 1980; GenBank No. V00141).

The nucleic acid of interest can be combined with or, where appropriate, can consist of a sequence encoding a selectable agent. Use may in particular be made of genes which confer resistance to an antibiotic such as hygromycin, kanamycin, bleomycin or streptomycin, or to herbicides such as glufosinate, glyphosate or bromoxynil. Preferably, said gene encoding a selectable agent is chosen from the bar gene (White et al. 1990; GenBank No. X17220) which confers resistance to the herbicide Basta® (glufosinate) and the NPTII gene which confers resistance to kanamycin (Bevan et al., 1983).

A vector, in particular a plasmid, containing at least one nucleic acid construct as described above is thus provided for implementing the invention.

The invention also relates to a cellular host, in particular a bacterium such as Agrobacterium tumefaciens, transformed with said vector. Such a cellular host can be used to transfect plant cells with a vector according to the invention.

The invention also relates to a plant cell transformed with a nucleic acid of interest linked to a targeting nucleic acid, the transcribed sequence of which is that of an mRNA of a nuclear gene, said mRNA being detectable in a plastid. Preferably, said mRNA is an mRNA of a nuclear gene selected from the group consisting of the genes represented in Table V and the eIF4E gene.

The transformation of plant cells can be carried out by transfer of a vector into protoplasts, in particular after incubation of the latter in a solution of polyethylene glycol (PG) in the presence of divalent cations (Ca²⁺) according to the method in the article by Krens et al. (1982).

The transformation of the plant cells can also be carried out by electroporation, in particular according to the method described in the article by Fromm et al. (1986).

The transformation of the plant cells can also be carried out by using a gene gun which makes it possible to project metal particles coated with DNA sequences of interest, at very high speed, thus delivering genes into the cell nucleus, in particular according to the technique described in the article by Finer et al. (1992).

Another method for transforming plant cells is that of cytoplasmic or nuclear microinjection.

Preferably, the plant cells are transformed with a vector by means of a cellular host which is itself transformed with said vector, the cellular host being capable of infecting said plant cells, thereby allowing the integration, into the genome of the latter, of the nucleic acid sequences of interest initially contained in the genome of the abovementioned vector.

Advantageously, the cellular host used is Agrobacterium tumefaciens, in particular according to the methods described in the articles by Bevan (1984) and by An et al. (1986), or alternatively Agrobacterium rhizogenes, in particular according to the method described in the article by Robaglia et al. (1987).

Preferably, the transformation of the plant cells is carried out by transfer of the T region of the tumor-inducing extrachromosomal circular Ti plasmid of Agrobacterium tumefaciens, using a binary system (Watson et al., 1994).

To do this, two vectors are constructed. In one of these vectors, the T-DNA region has been removed by deletion, with the exception of the right and left edges, a marker gene being inserted between them so as allow selection in the plant cells. The other partner of the binary system is an auxiliary Ti plasmid, which is a modified plasmid that no longer has any T-DNA, but still contains the vir virulence genes necessary for transformation of the plant cell. This plasmid is maintained in Agrobacterium.

The invention also relates to the production of transgenic plants that can be regenerated from the transformed plant cell, and also the transgenic plants thus obtained. The invention also comprises the plant cells and tissues, and the organs or parts of plants, including leaves, stems, roots, flowers, fruits and/or seeds, obtained from these plants.

Preferably, the plant cell according to the invention is a plant cell selected from the group consisting of maize, wheat, tomato, tobacco and rice.

### Method for Producing Proteins of Interest in Plastids

The method for targeting to a plastid according to the invention makes it possible to obtain the translocation of an RNA to a plastid and therefore a localized expression in the plastid of the protein optionally encoded by this RNA. The production of proteins in plant cell plastids is advantageous in terms of ease of extraction, but also stability, since certain proteases appear to be not very well represented in plastids, and in particular chloroplasts.

The invention therefore relates to a method for producing at least one protein of interest in a plastid of a plant cell, comprising the steps consisting in:

a) transforming a plant cell with a nucleic acid encoding a protein of interest linked to a targeting nucleic acid, the transcribed sequence of said targeting nucleic acid being that of an mRNA of a nuclear gene, said mRNA being detectable in a plastid of a plant cell; and

b) expressing said nucleic acid encoding a protein of interest.

Advantageously, said method of production contains an additional step consisting in extracting the proteins from the plastid by the usual methods known to those skilled in the art.

The plastid can be selected from the group consisting of a chloroplast, an amyloplast, a chromosplast, an etioplast, a gerontoplast and a proplastid. Said plastid is preferably a chloroplast.

Preferably, said mRNA detectable in a plastid is characterized by a concentration in a plastid that is greater than its cytoplasmic concentration. More preferably, the concentration of said mRNA in a plastid is at least twice its cytoplasmic concentration. The determination of the respective concentrations of the mRNA in the plastid and the cytoplasm can be carried out in accordance with the methods described in the present application.

More preferably, said targeting nucleic acid according to the invention has a DNA or RNA sequence, the transcribed sequence of which is that of an mRNA of a nuclear gene selected from the group consisting of the genes represented in Table V. Said nuclear gene is therefore selected from the group of genes having, as coding sequence, SEQ ID No.1, SEQ ID No.3, SEQ ID No.5, SEQ ID No.7, SEQ ID No.9, SEQ ID No.11, SEQ ID No.13, SEQ ID No.15, SEQ ID No.17, SEQ ID No.19, SEQ ID No.21, SEQ ID No.23, SEQ ID No.25, SEQ ID No.27, SEQ ID No.29, SEQ ID No.31, SEQ ID No.33, SEQ ID No.35, SEQ ID No.37, SEQ ID No.39 or SEQ ID No.41 identified in Arabidopsis thaliana and/or their homologous sequences in other species. According to a preferred embodiment, said targeting nucleic acid has a transcribed sequence which is that of an mRNA of the gene encoding the eukaryotic translation initiation factor eIF4E.

Advantageously, said nucleic acid encoding a protein of interest is fused to said targeting nucleic acid.

The nucleic acid encoding a protein of interest and the targeting nucleic acid can both be a DNA sequence. The nucleic acid encoding a protein of interest and the targeting nucleic acid can both be an RNA sequence.

The plant cell can be transformed with said nucleic acid encoding a protein of interest linked to a targeting nucleic acid in such a way as to obtain transient or stable expression of the protein of interest, preferably in such a way as to obtain stable expression.

The protein of interest can be a heterologous protein. The term “heterologous protein” is intended to mean a protein which is not expressed by the nontransformed plant cell. It can be a recombinant protein normally expressed in a eukaryotic organism, for example a protein of bacterial, human, animal or plant origin. It can in particular be a protein of agronomic interest, such as a protein which confers, on the plant, resistance to a herbicide (for example, Basta), a protein (a toxin or protease, for example) which confers, on the plant, resistance to pathogens such as insects, fungi, bacteria, viruses, etc., a protein which confers a capacity for fixing nitrogen or for increased photosynthesis, or a protein which confers increased resistance to drought, to salt or to extreme temperatures. It can also be a protein of industrial interest, such as an enzyme used in agrochemical processes. The protein can also be a protein of therapeutic and/or prophylactic interest, such as insulin, for example.

The invention is not limited to this method of production, and any method known to those skilled in the art can be envisioned. In particular, the use of an operon for producing the proteins in the plastid can be envisioned. An operon is the unit of expression and regulation of bacterial genes comprising structural genes and control elements in the DNA, recognized by products of regulatory genes. The invention also comprises the embodiment in which the RNA of interest is in the form of an operon-type RNA, giving several proteins, after translation in the plastid. This embodiment makes it possible to obtain the coordinated production of several proteins in the plastid, using a single construct. A system of protein production in the plastid comprising the lactose operon (inducible operon under negative control) can be used according to one embodiment of the invention.

### Method for Identifying RNAs Capable of Targeting an RNA of Interest to a Plastid

The inventors have shown that mRNAs, transcribed from nuclear genes, which were localized in plastids, and in particular which are present in the plastids with a concentration greater than their cytoplasmic concentration, make it possible to translocate an RNA sequence to which they are linked, from the nucleus and/or the cytosol of a plant cell to a plastid.

The invention therefore proposes a method for identifying an RNA capable of targeting an RNA of interest to a plastid of a plant cell, in which the concentration of a candidate RNA in a plastid and in the cytoplasm of a plant cell is determined, and where an RNA, the concentration of which in the plastid is greater than its concentration in the cytoplasm, is identified as an RNA capable of targeting an RNA of interest to a plastid of a plant cell.

Preferably, an RNA, the concentration of which in the plastid is at least twice its concentration in the cytoplasm, is identified as RNA capable of targeting an RNA of interest to a plastid of a plant cell.

In order to identify these RNAs of interest, methodology as described in example 1 can be used. It is, for example, possible to label a population of plastid RNAs and a population of total RNAs, each with cy3 and cy5, respectively. In order to compare the relative concentration of a gene X in these two populations, a dye swap can be carried out, i.e. the mixtures (population of plastid RNAs)−cy3+(population of total RNAs)−cy5 and (population of plastid RNAs)−cy5+(population of total RNAs)−cy3 can be respectively hybridized on two slides carrying an oligonucleotide specific for the gene X. The level of hybridization to the oligonucleotide is quantified by measuring the mean of the cy3 and cy5 fluorescence intensities, normalized by subtracting the local background noise, for the same RNA population (image acquisition using the ArrayScanner Generation III, Molecular Dynamics, and digitization of the images using the ImageQuant 5.2, Amersham Biosciences). The relative level of the mRNA of the gene X in the plastid RNAs compared with the total RNAs is then estimated by calculating the ratio of the mean fluorescence intensity in the plastid RNAs to the mean fluorescence intensity in the total RNAs. It is possible to identify, as an RNA of interest, an RNA for which the geometric mean of the mean fluorescence intensities, in the plastid and total RNA populations, is greater than −2, and for which the ratio of the mean fluorescence intensity in the total RNAs to the mean fluorescence intensity in the plastid RNAs is between 0 and 0.5.

The following examples illustrate the invention without limiting the scope thereof.

### EXAMPLES

### Example 1

### Identification of mRNA of Nuclear Genes which has a Plastidial Localization

Materials and Methods

Purification of Arabidopsis thaliana Chloroplasts

Crude chloroplasts were obtained from Arabidopsis thaliana leaves according to a method derived from the protocol described by Ferro et al. (Mol Cell Proteomics, 2003). All the processes were carried out at 0-5° C. in RNAse-free buffers.

Before the beginning of extraction, 6 tubes are prepared, containing 30 ml of a solution containing: 50% of Percoll, 0.4 M sorbitol, 20 mM tricine-KOH, 5 mM MgCl₂ and 2.5 mM EDTA. The Percoll gradients for purifying the chloroplasts are preformed by centrifugation at 38700 g for 55 min (Sorvall SS-34 rotor). The tubes containing these preformed Percoll gradients are stored at 0-5° C.

The plants (400-500 g of leaves) are placed in the dark at 4° C. for the overnight period preceding the extraction, washed with deionized water and then dried on filter paper before milling. The materiel (400-500 g of leaves per 2 liters of milling buffer containing: 0.4 M sorbitol, 20 mM tricine-KOH, pH 8.4, 10 mM EDTA, 10 mM NaHCO₃ and 0.1 mg/ml of defatted bovine serum albumin (BSA)) is milled twice for 2 seconds in a Waring Blendor at high speed. The milled material is filtered rapidly through 4-5 layers of gauze and one thickness of nylon blutex. The filtered solution is distributed equally into 6 centrifugation tubes (each 500 ml) and centrifuged at 2070 g for 2 min (Sorvall GS 3 rotor). The supernatant is removed and the pellets of organelles are taken up in a final volume of washing medium containing: 0.40 M sorbitol, 20 mM tricine-KOH, pH 7.6, 5 mM MgCl₂, 2.5 mM EDTA. The suspension of chloroplasts (6 ml per tube) is deposited onto the top of the preformed Percoll gradients. The gradients are centrifuged at 13,300 g for 10 min (Sorvall HB-6 swinging rotor). The intact chloroplasts (a dark green-colored band located in the lower part of the gradient) are recovered with a pipette. The suspension of intact chloroplasts is diluted 3-4-fold in 200-300 ml of washing buffer containing: 0.40 M sorbitol, 20 mM tricine-KOH, pH 7.6, 5 mM MgCl₂, 2.5 mM EDTA. The suspension is centrifuged at 2070 g for 2 min (Sorvall SS-34 rotor). Each pellet, containing the washed, purified and intact chloroplasts, is recovered for preparing the RNAs and/or preparing the stroma. At the end of this step, the intact chloroplast yield is 50 to 60 mg of proteins.

Verification of the Purity of the Purified Organelles

The purity of the purified chloroplasts is verified using various methods. (1)

enzymatic markers: for example, fumarase (EC 4.2.1.2), a marker for contamination with mitochondria; hydroxypyruvate reductase (EC 1.1.1.81), a marker for contamination with peroxisomes; (2) immunological markers: for example, antibodies directed against the T subunit of glycine-decarboxylase (a marker for contamination with mitochondria); antibodies directed against histone H3 (a marker for contamination with nuclei); (3) proteomic studies which have not made it possible to detect proteins derived from nuclei, mitochondria or the cytosol in the envelope of Arabidopsis plastids purified according to this protocol.

Purification of the Arabidopsis thaliana Chloroplast Stroma

All the processes were carried out at 0-5° C. in RNAse-free buffers. The intact chloroplasts purified from Arabidopsis thaliana leaves were lysed in a hypotonic medium containing 10 mM MOPS-NaOH, pH 7.8, 4 mM MgCl₂). The stroma was purified from the lysate by centrifugation on sucrose gradients (6 tubes, 13.2 ml, Ultraclear, Beckman) containing: 10 mM MOPS-NaOH, pH 7.8, 4 mM MgCl₂ in three layers of 0.3 M, 0.6 M and 0.93 M sucrose. The lysed chloroplasts (final volume adjusted to 21 ml) are deposited onto the top of the sucrose gradients (3.5 ml per tube). The tubes are centrifuged at 70000 g for 1 hour (Beckman SW41-Ti rotor). After this centrifugation step, the stroma, located at the top of the gradient, is taken for the nucleic acid extractions. At the end of this step, the stroma yield is approximately 30 mg of proteins.

Verification of the Purity of the Purified Stroma

The purity of the purified stroma is verified using various immunological markers: for example, antibodies directed against the E 37 protein or the ceQORH protein (markers for contamination with chloroplast envelope); antibodies directed against LHCP proteins (markers for contamination with thylakoids). These studies did not make it possible to detect envelope-derived proteins or thylakoids in the fractions of stroma purified according to this protocol.

Chloroplast RNA Extraction

A pellet of purified chloroplasts conserved at −80° C. is suspended, by homogenization on a vortex, in 7 ml of extraction buffer (50 mM Tris-HCl, pH 8, 300 mM NaCl, 2% SDS, 5 mM EDTA, pH 8, 0.5 mM aurintricarboxylic acid, 14.3 mM β-mercaptoethanol, 0.5% polyvinylpyrrolidone, MW 360 000) prepared extemporaneously, and then placed in a water bath at 65° C. for 15 min, with agitation every 2-3 minutes.

The solution is divided up and transferred into two tubes, and then centrifuged at 12 500 g at ambient temperature for 15 min. The supernatant is transferred into a new tube, to which 0.35 ml of 3M KOAc, pH 4.8, is added. The solution is homogenized and left in ice for 30 minutes, before centrifugation at 10000 g for 10 min at 4° C. (1) The supernatant is transferred into a new tube and 2 ml of phenol/chloroform/isoamyl alcohol (IAA) (25:24:1) are added. The solution is homogenized on a vortex for 2 to 3 min and then centrifuged at 4000 g for 15 min. Step (1) is repeated until homogenization, and then 2 ml of chloroform are added to the aqueous phase, and the solution is homogenized on a vortex for 2 to 3 min before centrifugation for 15 min at 4000 g. The two supernatants are transferred into a single tube containing 100 mg of PVPP, and then incubated for 20 min at 78° C., with gentle agitation every 2 to 3 minutes. The tube is then cooled on ice. 2.85 ml of water and 10.85 ml of chloroform/IAA (24:1) are added per 8 ml of supernatant, and the solution is then homogenized on a vortex for 2 to 3 min before centrifugation for 10 min at 4000 g. The supernatant is transferred into a tube and 4 ml of chloroform/IAA (24:1) are added, and the whole is homogenized on a vortex and then centrifuged for 10 min at 4000 g. The supernatant is transferred into a new tube and 8 ml of isopropanol are added. The whole is mixed and incubated at −20° C. for 12 h. After centrifugation at 5000 g for 45 min at 4° C., the pellet is washed with 70% ethanol, before being taken up in 500 μl of water and centrifuged at 10000 g for 5 min at 4° C. The supernatant is then transferred into an Eppendorf tube, to which 1 ml of water and 391 μl of 8M LiCl are added, and then left at 4° C. for 3 h. After centrifugation at 10000 g for 20 min at 4° C., the pellet is washed with 70% ethanol and then taken up in 100 μl of RNase-free water.

Stromal RNA Extraction

Approximately 1 ml of frozen stroma supernatant was transferred into a tube containing a mixture, preheated to 80° C., containing 2 ml of TLES buffer (100 mM Tris, pH 8, 100 mM LiCl, 10 mM EDTA, pH 8, 1% SDS, 1% PVPP, 1% PVP, 5 mM DTT) and 2 ml of phenol. After homogenization (2 min on a vortex) and centrifugation (15 min, 4000 g), the upper phase is removed. 1 ml of TLES buffer is added to the residual phenolic phase, and the mixture is then agitated and centrifuged. The upper phase is removed and combined with that previously put aside. An 8M LiCl solution is added so as to obtain a final LiCl concentration of 2M, and the RNA is thus precipitated overnight at 4° C. After centrifugation the pellet is taken up in 100 μl of water.

The RNA is purified using the Rneasy kit (Qiagen). According to the manufacturer's protocol, 350 μl of RLT buffer+3.5 μl of β-mercaptoethanol and 250 μl of absolute ethanol are added; the whole is homogenized and centrifuged for 15 seconds at 10000 rpm. 500 μl of RPE buffer are added to the membrane and the whole is centrifuged for 15 seconds at 10000 rpm. The eluate is removed and the column is washed again with 500 μl of RPE buffer, and the whole is centrifuged for 2 minutes at 10000 rpm a first time and then a second in order to remove the traces of ethanol. The RNA is eluted by adding 30 μl of RNase-free water to the column. After 1 minute, the whole is centrifuged for 1 minute at 10000 rpm. The elution is repeated with 30 μl of H₂O. The 2 eluates are combined and the solution is assayed.

Extraction of Leaf Total RNA

1 g of plant material is ground in liquid nitrogen. The powder obtained is transferred to a flask containing 2 ml of phenol and 2 ml of TLES buffer preheated to 80° C.; the whole is mixed on a vortex for 2 min before adding 2 ml of chloroform/isoamyl alcohol (C/IA) (24:1) and mixing again on a vortex for 2 min and then centrifuging for 12 min at 5000 g at 15° C. The supernatant is collected. 1 ml of TLES buffer is added to the remaining phenolic phase; mixing is carried out on a vortex for 2 min before centrifuging for 10 min at 5000 g at 15° C. The supernatant is again removed and combined with the first supernatant collected. One or more extractions with phenol/chloroform/isoamylic acid (25:24:1) can be carried out if a whitish interface between the aqueous phase and the phenolic phase is visible.

One volume of chloroform/isoamyl alcohol is added to the aqueous phase derived from the extraction with the phenol/chloroform/isoamyl alcohol mixture. Mixing is carried out on a vortex for 2 min before centrifuging for 10 min at 5000 g at 4° C. The supernatant is collected and the concentration of the solution is adjusted to 2M of LiCl with 8M LiCl. The RNA precipitates overnight at 4° C. The mixture is centrifuged for 45 min at 12000 rpm at 4° C., and the pellet is then resuspended with 100 μl of MilliQ H₂O for Mini RNA Clean Up (Qiagen).

The RNA is purified using the Rneasy kit (Qiagen). 350 μl of RLT buffer+3.5 μl of β-mercaptoethanol (added extemporaneously) and 250 μl of absolute ethanol are added, and the whole is homogenized and centrifuged for 15 seconds at 10000 rpm. 500 μl of RPE buffer are added to the membrane and the whole is centrifuged for 15 seconds at 10000 rpm. The eluate is removed and the column is washed again with 500 μl of RPE buffer; the whole is centrifuged for 2 minutes at 10000 rpm a first time and then a second in order to eliminate the traces of ethanol. The RNA is eluted by adding 30 μl of RNase-free water to the column. After 1 minute, the whole is centrifuged for 1 minute at 10000 rpm. The elution is repeated with 30 μl of H₂O. The 2 eluates are combined and the solution is assayed.

### Synthesis of the Cy3- or Cy5-Labeled Probes from the Total RNA

### Synthesis of the Cy3- or Cy5-Labeled Probe

3 μg of total RNA are placed in 8.5 μl of RNase-free H₂O. 0.5 μl of spike and 2 μl of random nonamers are added. The mixture is incubated for 10 minutes at 70° C. and then placed in ice for 1 min and centrifuged. The mixture is then incubated at ambient temperature for 10 minutes.

An incubation buffer, to be added to the RNA, is prepared, said buffer comprising, for one probe, 4 μl of 5×SSII buffer, 2 μl of 0.1 M DTT, 1 μl of a mixture of dNTP, 1 μl of dCTP Cy3 or Cy5, and 200 U of Superscript II (Invitrogen).

The incubation mixture is added to the RNA and incubated for 10 minutes at ambient temperature, and then for 3 hours at 42° C. 2 μl of 2.5 M NaOH are added. The whole is then incubated at 37° C. for 10 minutes, and then 10 μl of 2 M Hepes buffer, pH 8, are added.

### Purification of the Cy3- or Cy5-Labeled Probe

The probes are purified using the QIAGEN purification kit according to the supplier's protocol. Briefly, 500 μl of PB buffer are added to the probe. The mixture is loaded onto a column and then centrifuged for 2 min at 14000 rpm, the column is washed by adding 500 μl of PE washing buffer and then centrifuged for 1 min at 14000 rpm, the collecting tube is emptied, 500 μl of PE washing buffer are added and the whole is centrifuged for 1 min at 14000 rpm; the collecting tube is emptied and then 500 μl of PE washing buffer are added, the whole is centrifuged for 1 min at 14000 rpm, and the collecting tube is emptied and then centrifuged for 1 min at 14000 rpm in order to correctly dry out the column. The column is placed in a new collecting tube, and 50 μl of elution buffer are added to the membrane and left for 1 min at ambient temperature. The whole is centrifuged for 1 min at 14000 rpm. A second elution is carried out as previously, using 50 μl of elution buffer.

Preparation of Slides (Spotting)

The slides used for the hybridization are spotted beforehand using a robot (Lucidea spotter, Amersham Biosciences). The 26000 oligonucleotides (Operon 26K unigene set), each corresponding to one gene of the Arabidopsis genome, are distributed into 384-well plates, in denaturing solution, at a concentration of 2 μM. The 130 amplicons corresponding to the genes of the chloroplast genome and to the mitochondria transcripts are in denaturing solution at a concentration of 50 ng/μl. The whole of the Arabidopsis template (nuclear genome and organelles) is deposited onto type 7 Star glass slides (Amersham Bioscience). The slides are dried in the spotter chamber at a hygrometry of 50%, overnight. Each slide is then exposed to a UV at 500 mJ for 15 seconds (crosslinking).

Hybridization on Slide

Conventionally, when a microarray experiment is carried out and the level of expression in a sample A is compared in relation to a sample B, several technical repetitions (3) of a “dye swap” are carried out. A “dye-swap”, or inversion of fluorochromes, is a second hybridization experiment with the two fluorochromes being swapped in relation to the population. This therefore corresponds to two hybridizations on two different slides. The data derived from the hybridization of the two slides are usually processed together.

For a first conventional swap experiment, 6 tubes are prepared in the following way: 3× tube A containing 50 μmol Population A cy3+50 pmol Population B cy5, and 3× tube B containing 50 pmol Population B cy3+50 pmol Population A cy5. The probes are evaporated in a speed vac. Slide prehybridization: the slides are prehybridized in an extemporaneously prepared solution having the following composition: 5×SSC, 0.1% SDS, 0.1% BSA. The solution is placed at 42° C. for 2 hours and the slides are then soaked in the buffer at 42° C. with agitation for 45 min. The slides are rinsed in 3 successive baths of water and then dried in nitrogen.

6 sides which follow one another in the order of spotting of the same session of spotting are associated as follows with the probe tubes:

Position

| | N | N + 1 | N + 2 | N + 3 | N + 4 | N + 5 |
|---|---|---|---|---|---|---|
| Tube | A | A | A | B | B | B |

Treatment of cover slips: the cover slips are immersed in a solution of 1% SDS and incubated in a sonicator for 5 minutes. The cover slips are rinsed 5 times with milliQ water and then immersed in 70% EtOH. The cover slips are dried with nitrogen.

Hybridization: after evaporation, each probe (tube A or B) is taken up in 10.5 μl H₂O and 3 μl of fractionated hareng sperm DNA (0.1 mg/ml, Ci 1 mg/ml), and denatured for 2 minutes at 95° C., 30% formamide, 1Xhybridization buffer, Amersham Biosciences. The probes are denatured for 2 min at 95° C. and then kept in ice.

For the hybridization, the probe is deposited onto the cover slip and the slide covers the cover slip. The whole is placed in a Corning hybridization chamber and incubated in a water bath at 37° C. for 16 hours. The slides are washed with agitation in the following successive baths: 2×SSC 0.1% SDS for 5 min at 37° C., 2×SSC 0.1% SDS for 5 min at 37° C., 0.2×SSC for 1 minute a ambient temperature, 0.1×SSC for 1 min at ambient temperature, and then in water. The slides are dried with nitrogen and then scanned.

Image Acquisition

The optical reading of the chips is carried out using an ArrayScanner Generation III scanner (Molecular Dynamics) equipped with two lasers. These two lasers excite the two fluorescent molecules, Cy3 and Cy5, by emission of the two respective wavelengths of 550 nm and 649 nm. The photons emitted in return by the fluorochromes are captured by a photomultiplier (PMT) set at 700 V and transformed into an amplified electrical signal which is converted into two digital images in level of gray, one for each wavelength.

Image Processing

The digitalized images are visualized using the ImageQuant 5.2 software (Amersham Biosciences) in order to control their overall quality. Next, the ArrayVision 7.0 software (Amersham Biosciences) makes it possible to analyze the images and the method used provides, among other parameters, a value of the intensities measured for each spot and also the neighboring background noise. It is at this stage that the spots are annotated; the software assigns to each spot its coordinates and the identifier of the gene which corresponds thereto.

Normalization

For the comparisons RNAtotal_RNAchloro and RNAtotal_RNAstroma, it was chosen to carry out a swap normalization. In this case, 2 slides of a swap are associated and, for each intensity, the local background noise is subtracted. The mean of the intensities corresponding to the same RNA population is calculated. Since the background noise can sometimes be greater than the fluorescence value measured, the mean of the intensities measured for a population can be a negative value. The ratio of the 2 means is determined (RatioAB). When there are several technical replicates, a second ratio (ratioAB 2) is calculated. A factor A is also calculated, which factor is the geometric mean of the mean intensities measured in each of the two RNA populations compared (for example, A=\sqrt{IntRNAtotal*IntRNAchloro} if IntRNAtotal>0 and IntRNAchloro>0, or A=\sqrt{|IntRNAtotal*IntRNAchloro|} if IntRNAtotal<0 or IntRNAchloro<0).

For the RNAstroma_RNAchloro comparison, the normalization procedure is different. 3 technical repetitions or 3 swaps (6 slides) were carried out. The background noise was subtracted from the intensities and the 6 slides were normalized independently using the Loess method by block (Lonnstedt and Speed, 2002). For each slide, the stroma intensity/chloro intensity ratio is calculated and then converted to log₂. The mean of the 6 values of log₂ (ratio) is calculated and corresponds to M. The RatioAB is the ratio of the mean of the intensities corresponding to the RNAstroma to the mean of the intensities corresponding to the RNAchloroplast. A Bayesian statistical test (Yang et al., 2002) was applied in order to compare the 6 values of intensity corresponding to the RNA chloro population with the 6 values of intensity corresponding to the RNA stroma population. The stroma/chloro ratio is the mean of the 6 stroma/chloro ratios of each slide. T is the value of the statistical test, pvalue is the corresponding p value and B corresponds to the probability that the chloro/stroma ratio is other than 0 over the probability that the ratio is equal 1. When B is greater than 0, the gene has a greater probability of being differentially expressed than of being invariant.

Results

Comparison of the RNAtotal with the RNAstroma made it possible to identify the 1222 Arabidopsis genes listed in Table I. The genes selected met the following criteria: A>−2 for the 3 swaps, mean of the ratios RNAtotal_RNAstroma of the swaps of between 0 and 0.5 and coefficient of variation <0.1 (A=\sqrt{IntRNAtotal*IntRNAchloro}).

Comparison of the RNAchloro with the RNAtotal made it possible to identify the 1315 Arabidopsis genes that meet the following criteria: A>−2 for the 3 swaps, mean of the ratios RNAtotal_RNAchloro of the swaps of between 0 and 0.5 and coefficient of variation <0.1. This list of 1315 genes was crossed with the list of the 1222 genes previously selected and resulted in the selection of 683 common genes, which are listed in Table II.

An RNAstroma/RNAchloro comparison, with Loess block by block normalization, resulted in the selection of 109 genes (Table III, the expression of which is greater in the stroma compared with the chloroplast and with the total RNA, and the expression of which is greater in the chloroplast compared with the total RNA. These genes meet the criteria: value of the Bayesian statistical test >0 and M>0.

A list of 46 genes, shown in Table IV, was established by crossing two gene selections. The first selection of 287 genes was made from an RNAchloro/RNAtotal comparison on the basis of the following criteria: mean of the ratios AB of the swaps >1.5, variance <0.001 or ratio>5 if there is no variance threshold. The second selection of 706 genes was made from an RNAtotal/RNAstroma comparison on the basis of the following criteria: mean of the ratios of the 2 swaps <0.66, variance <0.001 or ratio>0.2 if there is no variance threshold. The 46 genes identified are the genes common to these two selections.

### Example 2

### Demonstration of Targeting of the mRNA of the Eukaryotic Transcription Initiation Factor 4E (eIF4E) to Chloroplasts

Materials and Methods

Analysis by Hybridization and Synthesis of RNA Probes

The in situ hybridization was carried out as described in Rodriguez et al.

(1998) with digoxigenin-labeled antisense sequences. The RNA probes and the Northern blotting analyses were carried out according to standard procedures (Sambrook et al., 1989). The cDNA probes were labeled by random priming using ³²P-dCTP and the RNA probes were labeled by in vitro transcription using either ³²P-UTP or digoxigenin (DIG RNA labeling kit, Roche diagnostics).

Chloroplast Purification and Nucleic Acid Extraction

All the processes were carried out at 0-5° C. The crude chloroplasts were obtained from leaves (6 g of A. thaliana, 30 g of N. tabacum, 100 g of L. sativa or 4 kg of S. oleracea). The plants were placed in the dark at 4° C. overnight and the chloroplasts were extracted in an isoosmotic buffer (A. thaliana and N. tabacum: TRIS-HCl, pH 8, 20 mM, EDTA, 0.33 M sorbitol, 0.1% β-mercaptoethanol; L. Sativa: 0.4 M sorbitol, 10 mM NaCl, 50 mM MOPS, pH 7; S. oleracea: 0.33 M sucrose, 20 mM MOPS, pH 7.8) and purified by isopycnic centrifugation on preformed Percoll gradients (Douce and Joyard, 1982). The N. tabacum chloroplasts were also obtained from protoplasts as described in Charbonnier et al. (1987).

The intact chloroplasts purified from S. oleracea were lysed in a hypotonic medium, and the stroma and the thylakoid and envelope membranes were purified from the lysate by centrifugation on sucrose gradients (Douce and Joyard, 1982). The chloroplast RNA or DNA of the subchloroplast fractions were extracted from the purified intact chloroplasts or from the subplastid fractions purified by extraction with phenol/chloroform and ethanol precipitation. For the Southern blotting analyses, the chloroplast nucleic acids were treated with RNAase and digested with the appropriate restriction enzymes.

Treatment of Purified Chloroplasts with RNAase and Protease

The intact protoplasts purified from N. tabacum were incubated with 50 μl of RNAase One (Promega) in the extraction buffer for 20 min on ice, before extraction of the RNA.

For the L. sativa chloroplasts, fifty nanograms of A thaliana cpSRP43 recombinant protein bearing a histidine tag (having a trypsin cleavage site downstream of the histidine tag) and 50 pg of AteIF4E antisense RNA (corresponding to the A thaliana eIF4E cDNA, which does not show any cross hybridization with the L. sativa eIF4E mRNA) labeled with digoxigenin were added to the purified L. sativa chloroplasts before incubation with trypsin (650 units). After incubation for 5 min on ice, 10 μg of RNAase A were added, before incubating for 4 min at ambient temperature. An aliquot of the mixture was mixed with the protein denaturing buffer for separate detection by Western blotting using an anti-histidine tag antibody. The RNA was purified from the rest of the incubation mixture and an aliquot was used for direct detection of the digoxigenin-labeled AteIF4E RNA after transfer onto a membrane. The rest of the RNA was used for Northern blotting hybridization with the digoxigenin-labeled LseIF4E antisense RNA probe.

Production of Transgenic Plants and Particle Bombardment

The AteIF4E1 cDNA was amplified by PCR and cloned upstream and in the reading frame of the green fluorescent protein 4 (mGFP5) gene under the control of the cauliflower mosaic virus (CaMV) 35S promoter (Von Arnim et al., 1998). The chimeric gene cassette was then placed in the binary vector pPZP-BASTA (a derivative of pPZP; Hajdukiewicz et al., 1994). The transformation of Arabidopsis with Agrobacterium was carried out in accordance with Bechtold et al. (1983). The particle bombardment using a pneumatic particle gun (Bio-Rad PDS-1000/He, helium pressure of 1550 psi, rupture disks 1350 psi, target distance 10 cm, 1 μm gold microbeads) and the observation by confocal laser microscopy (TCS-SP2, Leica, Deerfield, Ill.) were carried out as described in Ferro et al. (2002).

Results

The eIF4E1 mRNA is localized in the chloroplasts in four different plant species

The in situ hybridization experiments with an A thaliana eIF4E1 probe (AteIF4E1) show that the hybridization signal is associated with the chloroplasts. Since similarity searches in databanks had revealed an absence of sequence similarity between the AteIF4E1 mRNA and the chloroplast DNA of A. thaliana, the inventors sought to further characterize this observation.

A Northern blotting analysis was carried out on RNA extracted from chloroplasts purified from A. thaliana. The eIF4E mRNA can be detected in the chloroplast RNA extract with the AteIF4E1 antisense RNA probe. The low level of contamination of the chloroplast RNA preparation with cytosolic RNA was verified using a 28S rRNA probe. The AteIF4E mRNA was also detected specifically in the chloroplast RNA fraction by RT-PCR.

Chloroplast RNA was also purified from Nicotiana tabacum; it was thus observed that an N. tabacum e/F4E cDNA probe hybridized to the chloroplast RNA (NteIF4E). Contamination of the chloroplast RNA preparation with nuclear or cytosolic RNA was excluded using a probe for the U6 small nuclear RNA and a nitrite reductase (Nir) gene cDNA probe, respectively. A chloroplast probe, PsbB, detected the corresponding mRNA in all the extracts. In addition, treatment of the purified intact chloroplasts with RNAase, before the RNA extraction, does not cause the signal corresponding to the NteIF4E mRNA to disappear, thereby suggesting that the mRNA is protected from the RNAase activity either by protein complexes at the surface of the chloroplast, or by the envelope membranes of the intact chloroplasts.

In order to verify whether the eIF4E mRNA can bind to the outer membrane, chloroplasts were purified from Lactuca sativa (lettuce), which makes it possible to obtain better chloroplast yields than the purification from A. thaliana and from N. tabacum. The chloroplasts were treated with a combination of trypsin and RNAase in order to eliminate any RNA which could be protected by membrane-associated protein complexes. The hybridization with an L. sativa eIF4E probe (LseIF4E) showed that the LseIF4E mRNA was protected against the combined treatments with protease and RNAase. The effectiveness of these treatments was evaluated by adding, to the purified chloroplasts, exogenous recombinant protein labeled with a histidine tag (cpSRP43) and digoxigenin-labeled AteIF4E RNA. The disappearance of the cpSRP43 protein and of the AteIF4E RNA after treatment with the protease and the RNAse, although the LseIF4E mRNA was detected, showed that the LseIF4E mRNA is probably localized inside the chloroplast envelope. Control hybridization experiments revealed the absence of cross hybridization between the L. sativa eIF4E probe and the purified chloroplast DNA or the mitochondrial RNA.

Spinacia oleracea (spinach) was then used as a source of chloroplast in order to obtain the large amounts of chloroplasts necessary for fractionation into separate envelope, thylakoid and stroma fractions. The hybridization of the LseIF4E probe demonstrates that the homologous S. oleracea eIF4E (SoeIF4E) mRNA was localized in the chloroplast stroma, thereby excluding the localization of SoeIF4E in the intermembrane space of the chloroplast envelope and thus validating the delivery of the RNA through both the outer and inner membranes of chloroplast envelopes.

A Fusion of the eIF4E1 and GFP mRNAs is Delivered to Chloroplasts

The mRNA encoding GFP (Green Fluorescent Protein, mGFP5) was subsequently fused in the position 3′ of the AteIF4E1 mRNA under the control of the CaMV 35S promoter, and transgenic A. thaliana lines were produced. A line expressing the hybrid mRNA was selected and used to prepare chloroplast RNA. The hybridization with a GFP probe showed that the hybrid mRNA is effectively localized in the chloroplast fraction, as had been observed with the AteIF4E1 mRNA.

These results therefore demonstrate that the targeting of the eIF4E mRNA into chloroplasts takes place in four different plant species and therefore constitutes a general characteristic of plant cells. Furthermore, these results confirm the results observed on a chip, and demonstrate that an RNA preferentially detected as associated with the chloroplast is effectively translocated inside the plastid.

This is the first time that the importation of an endogenous RNA originating from another cellular compartment, into chloroplasts, is reported. The eIF4E protein is one of the key regulators of general and specific translation in eukaryotes (Gingras et al., 1999) but is not necessary for the translation of chloroplast mRNAs which lack the cap structure (Sugiura et al., 1998). A commonly observed method of regulation of translational activity in the cell is the sequestration of eIF4E by binding proteins (Gingras et al., 1999; Groisman et al., 2002). Since a large amount of proteins must be synthesized in the cytoplasm in a manner coordinated with the needs of chloroplasts, chloroplastic sequestration of eIF4E mRNA may be a means of regulating translational activity in the cytosol according to the physiological status of the chloroplast. RNA exchanges between the cytosol and the chloroplasts may constitute a new level of cellular integration in plants.

### BIBLIOGRAPHY

An G (1986). Development of plant promoter expression vectors and their use for analysis of differential activity of nopaline synthase promoter in transformed cells. Plant Physiol 81: 86-91
Bechtold N, Ellis J, Pelletier G (1993) In planta Agrobacterium mediated gene transfer by infiltration of adult Arabidopsis thaliana plants. C R Acad Sci Paris, Life Sciences 316, 1194-1199.
Bevan M. (1984) Binary Agrobacterium vectors for plant transformation. Nucleic Acid Research, 12(22):8711-21.
Charbonnier L, Primard C, Leroy P, Chupeau Y. (1987) A Miniscale method for the simultaneous isolation of chloroplast and mitochondrial DNA from tobacco, French bean and rapeseed. Plant Mol. Biol. Rep. 4, 213-218.
Choi S B, Wang C, Muench D G, Ozawa K, Franceschi V R, Wu Y, Okita T W. (2000) Messenger RNA targeting of rice seed storage proteins to specific ER subdomains. Nature. 407(6805):765-7.
Depicker A, Stachel S, Dhaese P, Zambryski P, Goodman H M. (1982) Nopaline synthase: transcript mapping and DNA sequence. J. Mol. Appl. Genet., 1, 561-573
Douce R, and Joyard J. in Methods in Chloroplast Molecular Biology (Edelman M, Hallick R, and Chua N H. eds.) Elsevier Science Publishers B.V. Amsterdam, pp. 239-256 (1982).
Ferro M, Salvi D, Riviere-Rolland H, Vermat T, Seigneurin-Berny D, Grunwald D, Garin J, Joyard J, Rolland N. (2002) Integral membrane proteins of the chloroplast envelope: identification and subcellular localization of new transporters. Proc Natl Acad Sci USA. 99:11487-92.
Ferro M, Salvi D, Brugière S, Miras S, Kowalski S, Louwagie M, Garin J, Joyard J & Rolland N (2003) Proteomics of the chloroplast envelope membranes from Arabidopsis thaliana. Mol. Cell. Proteomics 2: 325-345.
Finer, J. J., Vain, P., Jones, M. W. and McMullen, M. D. (1992) Development of the particle inflow gun for DNA delivery to plant cells. Plant Cell Reports 11:323-328.
Fischhoff D A, Bowdish K S, Perlak F J, et. al. (1987) Insect tolerant transgenic tomato plants. Bio/Technology, 5, 807-813.
Franck A, Guilley H, Jonard G, Richards K, Hirth L. (1980) Nucleotide sequence of cauliflower mosaic virus DNA. Cell, 21, 285-294 Fromm M E, Taylor L P, Walbot V. (1986) Stable transformation of maize after gene transfer by electroporation. Nature, 319: 791-793 Garcia I, Rodgers M, Lenne C, Rolland A, Sailland A and Matringue M (1997) Subcellular localization and purification of a p-hydroxyphenylpyruvate dioxygenase from cultured carrot cells and characterization of the corresponding cDNA. Biochem. J., 325:761-769.
Garcia I, Rodgers M, Pepin R, Hsieh T F, and Matringe M (1999). Characterization and subcellular compartmentation of recombinant 4-hydroxyphenylpyruvate dixoxygenase from Arabidopsis in transgenic tobacco. Plant Physiol, 119(4):1507-16.
Gingras A C, Raught B. and Sonenberg N. (1999) elF4 initiation factors: effectors of mRNA recruitment to ribosomes and regulators of translation. Annu. Rev. Biochem. 68, 913-63.
Groisman I, Jung M Y, Sarkissian M, Cao Q, Richter J D. (2002) Translational control of the embryonic cell cycle. Cell.; 109(4):473-83.
Hajdukiewicz, P., Svab, Z. & Maliga, P. (1994) The small, versatile pPZP family of Agrobacterium binary vectors for plant transformation. Plant Mol. Biol. 25, 989-94.
Hilder V A, Gatehouse A M R, Sheerman S E, Barker R F, Boulter D (1987) A novel mechanism of insect resistance engineered into tobacco. Nature, 300, 160-163.
Im K H, Cosgrove D J, Jones A M. (2000) Subcellular localization of expansin mRNA in xylem cells. Plant Physiol. 123(2):463-70.
Joyard J, Teyssier E, Miege, C, Berny-Seigneurin, D, Marechal, et al. (1998) The biochemical machinery of plastid envelope membranes. Plant Physiol. 118, 715-723.
Klee H J, Muskopf Y M, Gasser C S. (1987) Cloning of an Arabidopsis thaliana gene encoding 5-enolpyruvylshikimate-3-phosphate synthase: sequence analysis and manipulation to obtain glyphosate-tolerant plants. Mol. Gen. Genet., 210, 437-442.
Kloc M, Zearfoss N R, Etkin L D. (2002) Mechanisms of subcellular mRNA localization. Cell. 22; 108(4):533-44.
Krens F A, Molendijk L, Wullens G J and Schilperoort R^{A} (1982) In vitro transformation of plant protoplasts with Ti-plasmid DNA. Nature 296: 72-74
Lee K Y, Townsend J., Tepperman J., Black M., Chui C F, Mazur B. et al., (1988) The molecular basis of sulfonylurea herbicide resistance in tobacco. EMBO J., vol. 7, No. 5, pp. 1241-1248.
Lonnstedt I. et Speed T. P. (2002) Replicated Microarray Data. Statistical Sinica 12: 31-46.
Martin W and Herrmann R^{G}. (1998) Gene transfer from organelles to the nucleus: how much, what happens, and why. Plant Physiol. 118, 9-17
McElroy D., Blowers, A. D., Jenes, B. and Wu, R. (1991) Construction of expression vectors based on the rice actin 1 (Act1) 5′ region for use in monocot transformation. Mol. Gen. Genet. 231, 150-160.
Mitra A, Higgins D W. (1994) The Chlorella virus adenine methyltransferase gene promoter is a strong promoter in plants. Plant Mol. Biol. 26, 85-93,
Norris S R, Shen X, DellaPenna D (1998). Complementation of the Arabidopsis pdsl mutatin with the gene encoding p-hydroxyphenylpyruvate dioxygenase. Plant Physiol, 117(4):1317-23
Petracek M E, Dickey L F, Huber S C, Thompson W F. (1997) Light-regulated changes in abundance and polyribosome association of ferredoxin mRNA are dependent on photosynthesis. Plant Cell 9, 2291-300.
Preston C, Powles S B. (2002) Evolution of herbicide resistance in weeds: initial frequency of target site-based resistance to acetolactate synthase-inhibiting herbicides in Lolium rigidum. Heredity, 88(1), 8-13.
Robaglia C, Vilaine F, Pautot V, Raimond F, Amselem J, Jouanin L, Casse-Delbart F, Tepfer M. (1987) Expression vectors based on the Agrobacterium rhizogenes Ri plasmid transformation system. Biochimie. 69(3):231-7.
Rodriguez C M, Freire M A, Camilleri C, Robaglia C. (1998) The Arabidopsis thaliana cDNAs coding for eIF4E and elF(iso)₄E are not functionally equivalent for yeast complementation and are differentially expressed during plant development. Plant J. 13, 465-73.
Sambrook J, Fritsch E F. and Maniatis T. (1989) Molecular cloning: a laboratory manual, 2nd ed. Cold Spring Harbor Laboratory press.
Sharp, P. A. (2001) RNA interference—2001. Genes Dev, 15:485-490.
Sugiura M, Hirose T, and Sugita M. (1998) Evolution and mechanism of translation in chloroplasts. Annu. Rev. Genet. 32, 437-59.
Surpin M, Larkin R M, Chory J. (2002) Signal transduction between the chloroplast and the nucleus. Plant Cell 14: 327-338.
Vaeck, M., A. Reynaerts, H. Hofte, S. Jansens, M. DeBeuckeleer, C. Dean, M. Zabeau, M. Van Montagu, and J. Leemans. (1987) Transgenic plants protected from insect attack. Nature 328: 33-37.
Van Haaren M J, Houck C M. (1991) Strong negative and positive regulatory elements contribute to the high-level fruit-specific expression of the tomato 2A11 gene. Plant Mol. Biol. 17, 615-630, 1991.
van Heerden A, Browning K S. (1994) Expression in Escherichia Coli of the two subunits of the isozyme form of wheat germ protein synthesis initiation factor 4F. Purification of the subunits and formation of an enzymatically active complex. J. Biol. Chem. 269:17454-17457.
Verdaguer B, de Kochko A, Fux Cl, Beachy R N, Fauquet C. (1998) Functional organization of the cassaya vein mosaic virus (CsVMV) promoter. Plant Mol. Biol., 37(6):1055-67.
Von Arnim, A. G., Deng, X. W. & Stacey, M. G. (1998) Cloning vectors for the expression of green fluorescent protein fusion proteins in transgenic plants. Gene. 221, 35-43.
Watson et al. (1994) Ed. De Boeck Université, pp 273-292
White J., Chang S-YP., Bibb M J. and Bibb M J. (1990) A cassette containing the bar gene of Streptomyces hygroscopicus: a selectable marker for plant transformation. Nucl. Acid. Res. 18, 1062.
Yang, Y. H., Dudoit, S., Luu, P., Lin, D. M., Peng, V., Ngai, J., and Speed, T. P. (2002). Normalization for cDNA microarray data: a robust composite method addressing single and multiple slide systematic variation. Nucleic Acids Research 30(4):e15.

1. A method for targeting an RNA of interest to a plastid of a plant cell, said method comprising the transformation of a plant cell with a nucleic acid of interest linked to a targeting nucleic acid, the transcribed sequence of which is that of an mRNA of a nuclear gene, said mRNA being detectable in a plastid.

2. The method as claimed in claim 1, in which the targeting nucleic acid has, as transcribed sequence, that of an mRNA of an endogenous nuclear gene.

3. The method as claimed in claim 1, in which said plastid is selected from the group consisting of a chloroplast, an amyloplast, a chromoplast and a proplastid.

4. The method as claimed in claim 1, in which said mRNA is characterized by a concentration in a plastid which is greater than its cytoplasmic concentration.

5. The method as claimed in claim 4, in which said mRNA is characterized by a concentration in a plastid that is at least twice its cytoplasmic concentration.

6. The method as claimed in claim 1, in which said targeting nucleic acid has a transcribed sequence which is that of an mRNA of a gene selected from the group consisting of the genes having, as coding sequence, SEQ ID No.1, SEQ ID No.3, SEQ ID No.5, SEQ ID No.7, SEQ ID No.9, SEQ ID No.11, SEQ ID No.13, SEQ ID No.15, SEQ ID No.17, SEQ ID No.19, SEQ ID No.21, SEQ ID No.23, SEQ ID No.25, SEQ ID No.27, SEQ ID No.29, SEQ ID No.31, SEQ ID No.33, SEQ ID No.35, SEQ ID No.37, SEQ ID No.39 and SEQ ID No.41.

7. The method as claimed in claim 1, in which said targeting nucleic acid has a transcribed sequence which is that of an mRNA of a gene encoding the eukaryotic translation initiation factor eIF4E.

8. The method as claimed in claim 1, in which said nucleic acid of interest is a DNA.

9. The method as claimed in claim 1, in which said nucleic acid of interest is an RNA.

10. The method as claimed in claim 1, in which said targeting nucleic acid is a DNA.

11. The method as claimed in claim 1, in which said targeting nucleic acid is an RNA.

12. The method as claimed in claim 1, in which said nucleic acid of interest encodes a heterologous protein.

13. A nucleic acid construct comprising a nucleic acid of interest linked to a targeting nucleic acid, the transcribed sequence of which is that of an mRNA of a nuclear gene selected from the group consisting of the genes having, as coding sequence, SEQ ID No.1, SEQ ID No.3, SEQ ID No.5, SEQ ID No.7, SEQ ID No.9, SEQ ID No.11, SEQ ID No.13, SEQ ID No.15, SEQ ID No.17, SEQ ID No.19, SEQ ID No.21, SEQ ID No.23, SEQ ID No.25, SEQ ID No.27, SEQ ID No.29, SEQ ID No.31, SEQ ID No.33, SEQ ID No.35, SEQ ID No.37, SEQ ID No.39 and SEQ ID No.41.

14. A plant cell transformed with a nucleic acid of interest linked to a targeting nucleic acid, the transcribed sequence of which is that of an mRNA of a nuclear gene selected from the group consisting of the genes having, as coding sequence, SEQ ID No.1, SEQ ID No.3, SEQ ID No.5, SEQ ID No.7, SEQ ID No.9, SEQ ID No.11, SEQ ID No.13, SEQ ID No.15, SEQ ID No.17, SEQ ID No.19, SEQ ID No.21, SEQ ID No.23, SEQ ID No.25, SEQ ID No.27, SEQ ID No.29, SEQ ID No.31, SEQ ID No.33, SEQ ID No.35, SEQ ID No.37, SEQ ID No.39 and SEQ ID No.41.

15. A transgenic plant that can be regenerated from the transformed plant cell as claimed in claim 14.

16. A method for producing at least one protein of interest in a plastid of a plant cell, the method comprising the steps consisting in:
a) transforming a plant cell with a nucleic acid encoding a protein of interest linked to a targeting nucleic acid, the transcribed sequence of said targeting nucleic acid being that of an mRNA of a nuclear gene, said mRNA being detectable in a plastid of a plant cell; and
b) expressing said nucleic acid encoding a protein of interest.

17. The method as claimed in claim 16, in which the transcribed sequence of said targeting nucleic acid is that of an mRNA of a nuclear gene, said mRNA being characterized by a concentration in a plastid that is greater than its cytoplasmic concentration.

18. A method for identifying an RNA capable of targeting an RNA of interest to a plastid of a plant cell, in which the concentration of a candidate RNA in a plastid and in the cytoplasm of a plant cell is determined, and where an RNA, the concentration of which in the plastid is greater than its concentration in the cytoplasm, is identified as RNA capable of targeting an RNA of interest to a plastid of a plant cell.

19. The method as claimed in claim 18, in which an RNA, the concentration of which in the plastid is at least twice its concentration in the cytoplasm, is identified as RNA capable of targeting an RNA of interest to a plastid of a plant cell.


SEQUENCE LISTING (42 sequences)

SEQ ID NO: 1 - DNA, 1653 bp, Arabidopsis thaliana
  CDS (1)..(1653)
atggcgaattctcaaagatcatcatctctgatcgatccaaggaacggtttctgcacatcaaactccacattttacagtaaacgcaaaccattggcacttccttcaaaagaatcactcgacatcacaacattcatctcctcccaaacgtaccgtggcaaaaccgccttcatagacgcagccacggaccaccgcataagcttctccgatttatggatggccgtggatcgtgtcgccgactgtctcctccacgacgtgggaatacggagaggtgacgtggtcctagtcctctctcccaacaccatctccatccccattgtctgcctctccgtgatgtctcttggcgctgtcttaactaccgctaatcctctcaacaccgctagtgaaatcttaagacaaatcgcggatagcaatccaaaactcgccttcacaacaccggaactggctcccaaaatcgccagttccggtatctctatcgtcctcgagcgcgtagaagacactctacgcgttccgagaggactcaaagtggttgggaatctaactgaaatgatgaagaaagaaccaagtggacaagcagtcagaaaccaagttcataaagacgacacggcgatgttgctttactcctcggggacgacgggacgaagcaaaggagtgaattcgtctcacgggaacttaatagcacacgtggcgagatacatcgcggagccattcgagcagccacaacagaccttcatttgcactgttccgttgttccacacttttggtttactaaacttcgttttggccaccttagcgttaggtacgaccgttgtcatcctcccgaggtttgacctcggagagatgatggcggctgtcgagaaatacagagcgacgactctgattctcgtgccgccagttttagtaactatgataaacaaagcggatcaaataatgaagaagtacgacgtgagcttcttgagaacggtgcggtgcggtggagcacctttgagcaaggaagttactcaagggtttatgaagaaatatccaacggttgatgtttatcaaggatacgcattgacggaatctaacggtgcaggagcttcgatagaatcagtggaggagagtcggaggtacggtgcggtggggttgttgtcatgtggtgtagaagcgaggattgtggatccgaatacgggtcaggtcatgggtttgaaccaaacgggcgaactttggcttaaagggccttctatcgccaaaggttatttcaggaatgaagaagaaattataacttcagaaggatggcttaaaacaggagatctatgctatatagacaacgatggatttctttttatcgttgatcgattgaaagagcttatcaaatacaaaggttaccaggtacctccagctgaactagaggctctcttactaaaccatccagatattctcgatgcagccgttattccgtttccagataaagaagcaggacaatttccgatggcttacgtagcacgaaagcctgagagtaatctttgtgagaaaaaggttatcgactttatttctaaacaggtggcaccatataagaaaataagaaaagtcgcatttatagactctataccaaagactccatcgggcaaaacacttcgcaaggatctaatcaaatttgccatttcaaaaatttaa

SEQ ID NO: 2 - protein, 550 aa, Arabidopsis thaliana
MANSQRSSSLIDPRNGFCTSNSTFYSKRKPLALPSKESLDITTFISSQTYRGKTAFIDAATDHRISFSDLWMAVDRVADCLLHDVGIRRGDVVLVLSPNTISIPIVCLSVMSLGAVLTTANPLNTASEILRQIADSNPKLAFTTPELAPKIASSGISIVLERVEDTLRVPRGLKVVGNLTEMMKKEPSGQAVRNQVHKDDTAMLLYSSGTTGRSKGVNSSHGNLIAHVARYIAEPFEQPQQTFICTVPLFHTFGLLNFVLATLALGTTVVILPRFDLGEMMAAVEKYRATTLILVPPVLVTMINKADQIMKKYDVSFLRTVRCGGAPLSKEVTQGFMKKYPTVDVYQGYALTESNGAGASIESVEESRRYGAVGLLSCGVEARIVDPNTGQVMGLNQTGELWLKGPSIAKGYFRNEEEIITSEGWLKTGDLCYIDNDGFLFIVDRLKELIKYKGYQVPPAELEALLLNHPDILDAAVIPFPDKEAGQFPMAYVARKPESNLCEKKVIDFISKQVAPYKKIRKVAFIDSIPKTPSGKTLRKDLIKFAISKI

SEQ ID NO: 3 - DNA, 1728 bp, Arabidopsis thaliana
  CDS (1)..(1728)
atgggtaattgctgcggaagtaaacccctaacagcttctgatatcgtctcggatcaaaagcaagagacgattcttgggaagccattggaagatatcaagaagcactatagcttcggtgacgaactaggcaaaggaaagagttacgcttgcaaatccatacctaagagaactctaagcagcgaagaagaaaaagaagctgtgaagacagagattcaaatcatggatcatgtttcaggacagcctaacatcgtacagatcaaaggttcctacgaagataataattctatacacattgtaatggaattgtgtggtggtggtgagttattcgacaagatcgatgctttggtcaagtctcatagctattactctgagaaagatgctgctggaatctttaggtctattgtgaatgctgtgaagatttgtcattctttggatgtcgttcatcgtgatctcaagcctgagaacttcttgttctctagtaaagatgagaatgctatgcttaaagctatcgatttcgggtgttctgtttacatcaaagaagggaaaacttttgagagagttgttggaagtaaatactacattgctcctgaagtattagagggaagctatgggaaagaaatcgacatttggagtgctggtgttattttatacatcctactgagcggcgtaccgccatttcaaactggaattgaatctataattgtgtctactctttgtattgtagatgctgagattaaagaatgcaggcttgattttgagagccaaccatggcctttgatatcttttaaagcgaagcaccttatcgggaagatgctcaccaaaaaaccgaaggaacgaatctctgctgcagatgttcttgaacatccatggatgaaaagcgaagctccagataagcctattgataatgttgtcttatcacgtatgaagcaattccgagcaatgaacaagcttaagaagcttgctcttaaggttattgcggagggtctatcggaagaggagatcaaaggtcttaaaaccatgtttgagaatatggatatggacaaaagcgggtcaatcacttatgaagaactcaaaatggggctgaatagacatggctctaaactctctgagactgaagttaagcaactcatggaagcagtgagtgctgatgttgatgggaatggaacaattgactacatagagtttatctcagcgacgatgcatagacaccgtttggaacgagatgaacatttatacaaagcattccaatactttgataaagacggaagcgggcacataacgaaggaggaagtggagatagcaatgaaagagcatggtatgggagatgaagctaatgccaaagatttgatttcagaattcgataaaaacaacgatggaaaaatagactatgaggagttttgtacgatgatgagaaatggcatcttgcaaccacaagggaaacttttaaaacggctctatatgaatcttgaagaactcaaaactgggctaactagacttgggtctagactctctgaaactgaaatcgacaaagcattccaacactttgataaagacaacagcgggcacataactagagatgagttggaaagtgcaatgaaagaatatggaatgggagatgaagctagcatcaaagaagttatatccgaagttgataccgacaatgtaagttgcactctccaacatatagccaacatctcaaacattaaacaggttcttgaaaccttgtaa

SEQ ID NO: 4 - protein, 575 aa, Arabidopsis thaliana
MGNCCGSKPLTASDIVSDQKQETILGKPLEDIKKHYSFGDELGKGKSYACKSIPKRTLSSEEEKEAVKTEIQIMDHVSGQPNIVQIKGSYEDNNSIHIVMELCGGGELFDKIDALVKSHSYYSEKDAAGIFRSIVNAVKICHSLDVVHRDLKPENFLFSSKDENAMLKAIDFGCSVYIKEGKTFERVVGSKYYIAPEVLEGSYGKEIDIWSAGVILYILLSGVPPFQTGIESIIVSTLCIVDAEIKECRLDFESQPWPLISFKAKHLIGKMLTKKPKERISAADVLEHPWMKSEAPDKPIDNVVLSRMKQFRAMNKLKKLALKVIAEGLSEEEIKGLKTMFENMDMDKSGSITYEELKMGLNRHGSKLSETEVKQLMEAVSADVDGNGTIDYIEFISATMHRHRLERDEHLYKAFQYFDKDGSGHITKEEVEIAMKEHGMGDEANAKDLISEFDKNNDGKIDYEEFCTMMRNGILQPQGKLLKRLYMNLEELKTGLTRLGSRLSETEIDKAFQHFDKDNSGHITRDELESAMKEYGMGDEASIKEVISEVDTDNVSCTLQHIANISNIKQVLETL

SEQ ID NO: 5 - DNA, 1764 bp, Arabidopsis thaliana
  CDS (1)..(1764)
atgttgccaaagtttgatctaacagacccaaaagctagcctgtctcttcttgaggatgtgaccaccaacgtaacgcagattcaagattccatcttggaagcagtactttcacgtaatgctcataccgagtatcttaaaggtttcctcaacggtcaagttgataagcaaaccttcaagaagaacgtacccattgtgacctatgaagatattaagccttacatcaatcgtatcgctaatggagaggcgtctgatctcatctgtgatcgacccatcagtttacttgtgatgagctctggtactacagcaggaattcaaaatttgattcctttgacaacagaggatggggaacagaggattatgtttggatctctctatagatctttactctataagtacgtcgaggggattagagaaggaaagtctctcacgttctatttcgtgaaccctgaaagagagactgcctctgggatactgatcaggactatgataacttgtattttgaaaagcgtaaacaaaaccaactcatctctttgggatagattacagataagcccgcatgaaatttctacttgtgaagacactactcagagcatgtactgtcaattgctttgtgggcttctacaacgagataatgtagctcgcctcggtgcaccctttgcttctgtcttcatcagagtaatcaagtacttggaaggtcattggcaagagttatgctcaaacataaggactggccgtctcagtgactggatcacagaccctcaatgtgtctcagggattagtaagttcctcaccgctccaaatccggacctagcgagccttatcgagcaagaatgcagtaaaacctcatgggaggcaatagtgaagagactttggccaaaagcaaaatgcgtcgaagccgtcgttacaggctccatggcacagtacattccgttgctggaattctatggcggtggtcttccgttgatttcatcgtggtatggcagctctgaatgtttcatgggtgtcaatgtcaatcctctgtgcaagcctagcgatgtgtcgtacaccatcatcccatctatggcgtacttcgagttcttagaggtcaagaaagaccaacaagaagctggtcttgatcccatagagaaccatgtggtcgtcgatcttgtcgatgttaaaattggccatgattatgaacctgtcgtcacaacgttttctggtctgtataggtaccgtgtgggtgatcttttaagagttactggtttctacaacaattcaccacatttccgtttcgtgggaagacagaaagttgttctgagcctccacatggccaatacatacgaagaagacctccttaaggcagtgacaaacgcaaagctcctgctcgagccacatgacctgatgctaatggagttcactagccgtgtggattcgtcctcgtttgtaggacactatgtgctctattgggaacttgggagcaaagtcaaggacgccaagctcgaacctaaccgcgatgttatggaagaatgttgcttcaccgttgagaagtatctggaccctctctatagacaagaacgaagaaaagataagaacattggacctcttgagattaaggttgttaagcctggcgccttcgacgaactcatgaatttctttctgtctcgaggctcttctgtgagtcagtacaagacgccgaggtcggtgaagactgaagaagccgtcaagatattggaggccaacgttgtttccgagtttcttagtcaggaaactccgccgtggggataa

SEQ ID NO: 6 - protein, 587 aa, Arabidopsis thaliana
MLPKFDLTDPKASLSLLEDVTTNVTQIQDSILEAVLSRNAHTEYLKGFLNGQVDKQTFKKNVPIVTYEDIKPYINRIANGEASDLICDRPISLLVMSSGTTAGIQNLIPLTTEDGEQRIMFGSLYRSLLYKYVEGIREGKSLTFYFVNPERETASGILIRTMITCILKSVNKTNSSLWDRLQISPHEISTCEDTTQSMYCQLLCGLLQRDNVARLGAPFASVFIRVIKYLEGHWQELCSNIRTGRLSDWITDPQCVSGISKFLTAPNPDLASLIEQECSKTSWEAIVKRLWPKAKCVEAVVTGSMAQYIPLLEFYGGGLPLISSWYGSSECFMGVNVNPLCKPSDVSYTIIPSMAYFEFLEVKKDQQEAGLDPIENHVVVDLVDVKIGHDYEPVVTTFSGLYRYRVGDLLRVTGFYNNSPHFRFVGRQKVVLSLHMANTYEEDLLKAVTNAKLLLEPHDLMLMEFTSRVDSSSFVGHYVLYWELGSKVKDAKLEPNRDVMEECCFTVEKYLDPLYRQERRKDKNIGPLEIKVVKPGAFDELMNFFLSRGSSVSQYKTPRSVKTEEAVKILEANVVSEFLSQETPPWG

SEQ ID NO: 7 - DNA, 783 bp, Arabidopsis thaliana
  CDS (1)..(783)
atggaaactacaaaaaacactaatctccaaaaccctactaaactcccaaaaccatttcaacatcacctagaagaagaaaaagaagacgcactatctctccgagatcttcccctcaaagccaagaatcctaatcccaccaccaccgaagaccacaaagaaccgtcaacggaactttttgagtttctaacctcatcctcctacgacgtagctccggcagaaaatataatctttggcgggaaactcatccctttaaactaccaaaatgctttcttttcacctcctgagcacattagccgtcgaatccgttcacggtctgagtccttatccgcgatacaaggccataagcttaaccgtcccggtagttgtaccgtggcacgtcgtgacaatgcgggacctatgagggcaagccgctctttagattatcgtaagctttcacgtggtctaaccaccgtacattctccaccggaaaatagttcatcgactaaaaacacggggaagcctgaaacaacgtcttcgggaagtgtaaaaagtgtaaggccaagatggtatgtgatcatgtttggaatggttaagtttccgccggagatcgaactcaaggatataaagagccgtcagattcgccggaatattccaccggttatgtttccatctcctgctaacaggagagctcgtggatctcggtcgccatcaccgtcaccttcttggaggtttctcaacgctttaagttgcaagaagcccacaagcgtggctgctacggcgccgttttgggttcctcatccataa

SEQ ID NO: 8 - protein, 260 aa, Arabidopsis thaliana
METTKNTNLQNPTKLPKPFQHHLEEEKEDALSLRDLPLKAKNPNPTTTEDHKEPSTELFEFLTSSSYDVAPAENIIFGGKLIPLNYQNAFFSPPEHISRRIRSRSESLSAIQGHKLNRPGSCTVARRDNAGPMRASRSLDYRKLSRGLTTVHSPPENSSSTKNTGKPETTSSGSVKSVRPRWYVIMFGMVKFPPEIELKDIKSRQIRRNIPPVMFPSPANRRARGSRSPSPSPSWRFLNALSCKKPTSVAATAPFWVPHP

SEQ ID NO: 9 - DNA, 936 bp, Arabidopsis thaliana
  CDS (1)..(936)
atgggagacaacaaccctaaccgatcagaagcagaacgtcttctcggaatcgcggagaagcttctcgagtcacgagatctaaacggttcaaaagagtttgcaatcttagctcaagagacagagccactcctcgaaggcaccgatcaaatcctcgccgtcgtcgatgtcttactctcatcagcaccagagaatcgtatcaaaaaccaaccaaactggtacaaaatccttcagatcgaagatctaactgaatcatcaacagacaacgatctaatcaagaaacaataccgtcgtcttgctcttcttctccaccctgacaaaaaccgtttccctttcgccgatcaagctttcagattcgtgcttgatgcatgggaagttctatcaacacctacgaagaaatctcaattcgatggagatttgaatctcatcttcactaaagtaaatctcaacactcagaaatcgaagaagaaaacaacaacgaatgagaagatgtctacgttttggacggcgtgtccgtactgttacagtcttcatgagtatcctagggtttatcaagagtattgtattagatgtcaaaactgtcaaagagcgtttcacgctgcgagtattcctcagttgcctccgttgatacctggtaaagatgagtattattgttgttggggtttttttccgatggggtttgttggtggtaaaggaggagaagctgccattgctaatggagtagatgcagctaagttccctaattggatgcctccggttttctcatccggcggcgttgcagctcctccaagtggtaatggtgttagttttgatggatggtcaggtggtgcggcgaagagagataatgaggctgtgaggagtaataatggtgttggagttaattcagatggaacaccgaagaagagaggaagaggaaggccgaagaagaatccggtttag

SEQ ID NO: 10 - protein, 311 aa, Arabidopsis thaliana
MGDNNPNRSEAERLLGIAEKLLESRDLNGSKEFAILAQETEPLLEGTDQILAVVDVLLSSAPENRIKNQPNWYKILQIEDLTESSTDNDLIKKQYRRLALLLHPDKNRFPFADQAFRFVLDAWEVLSTPTKKSQFDGDLNLIFTKVNLNTQKSKKKTTTNEKMSTFWTACPYCYSLHEYPRVYQEYCIRCQNCQRAFHAASIPQLPPLIPGKDEYYCCWGFFPMGFVGGKGGEAAIANGVDAAKFPNWMPPVFSSGGVAAPPSGNGVSFDGWSGGAAKRDNEAVRSNNGVGVNSDGTPKKRGRGRPKKNPV

SEQ ID NO: 11 - DNA, 189 bp, Arabidopsis thaliana
  CDS (1)..(189)
atgggaaaggtccacggttcattggctcgtgccggtaaggtgagaggtcagacacctaaagtggctaagcaggacaagaagaagaagcctcgtggtcgtgctcataagcgtctgcaacacaaccgccgtttcgtcaccgccgttgttggttttggcaagaagagaggaccaaactcatcagagaagtag

SEQ ID NO: 12 - protein, 62 aa, Arabidopsis thaliana [sequence omitted]

SEQ ID NO: 13 - DNA, 747 bp, Arabidopsis thaliana
  CDS (1)..(747)
atgaagcgtgagtatttagattatacacttgtgcctttagggcttgctttgatggtcttttatcatctatggcttctctaccgtatcatccaccgtccttcttcgactgttgttggccttaacgccttcaatcgccgcctttgggtccaagccatgatggaggactcatccaaaaacggcgttttagcggttcaaacgctaaggaacaacataatggcatcaactctattagcatcaaccgcaataatgttatgttcactaatcgcggtactaatgactagcgctacaggagagcgatcagtctggtttgtgtttggagacaaaagcgaccgagccttctcgctcaagttcttcgcaatcctcgtgtgtttcctagtcgcgtttctcctcaatgtccaatcaattcgttattacagccatgcaagcatccttatcaatgtcccctttaaacagctcatggctgtttcttcaggtggtcgtggtaatggtagtcttatgataaaccaagattatgtggctgcaacggttaaccgtgggagttacttctggtcgttaggactacgagcgttctacttctcctcgcctttattcctatggatctttggtcccatcccaatgttcataacttgttgcgttcttgtttgttcgttgtacttccttgatttaacgtttgactcgatgaaatgtagcgttggagccgcagacgcggaggagcccgagattagaagcttagctcagaatgtttag

SEQ ID NO: 14 - protein, 248 aa, Arabidopsis thaliana
MKREYLDYTLVPLGLALMVFYHLWLLYRIIHRPSSTVVGLNAFNRRLWVQAMMEDSSKNGVLAVQTLRNNIMASTLLASTAIMLCSLIAVLMTSATGERSVWFVFGDKSDRAFSLKFFAILVCFLVAFLLNVQSIRYYSHASILINVPFKQLMAVSSGGRGNGSLMINQDYVAATVNRGSYFWSLGLRAFYFSSPLFLWIFGPIPMFITCCVLVCSLYFLDLTFDSMKCSVGAADAEEPEIRSLAQNV

SEQ ID NO: 15 - DNA, 189 bp, Arabidopsis thaliana
  CDS (1)..(189)
atgggaaaggttcacggatctttggctcgtgccggtaaggttagaggacaaactccaaaggttgcgaagcaagataagaaaaagaagcccagaggtcgtgctcacaagcgtctgcagcacaaccgtcgttttgtcaccgcagttgttggtttcggaaagaagagaggacccaactcttctgagaaatag

SEQ ID NO: 16 - protein, 62 aa, Arabidopsis thaliana [sequence omitted]

SEQ ID NO: 17 - DNA, 1119 bp, Arabidopsis thaliana
  CDS (1)..(1119)
atgtctaacgccgacgaaccaccgcagaaaaccaaccaaccaccgtcgtcttcattgacgccgccgtcattgttctcactcccggtggacatcgtattgaacatcttagccttggtaccaaaacgatattacccaatcctctgttgcgtctccaagtccttgcggagtctaattcgctcgccggagattcacaagacgcgatctttacacggaaaggactctctttatctatgcttctcgaccagaactacatacccgaaccggaaccggacgacttttcactggttcactctccggaggaatgataacaagatgaatacgactgagaatgtcttcgtctccatcgatgttccttatcgtcccgggcatgcttcttaccctctttccaatatcgcaattgatacagagatttactgcattcccggatataattttccctcatcatcaatcgtatggatctttgacacccaatctggacagtggcgtcaaggacctagtatgcaagtggagcgattatcagctaccgtaggactagtcggtggtaagatctatgtgatcggaggcaacagaggcgaagagatcctagcggaagtatttgacttaaagacacaaacttgggaagcagcgccgattcctaaggcaaaggatagaaacgaatggttcacgcatgcaagtgtttcacttgataggaaggtttatgctttaaattcgagggaatacatgaattcttacgatactagagatggttcttatcaaagatatacgatacctgaagataattggtggaagacaggaaaatgcgtgattgacaatgtgctcttcgtttacttccttagatttgggttaatgtggtatgactctgagctgatgttgtggagagtggtttatggtttagatcttgacaaagctcgatgcattgggatcggggaatactacggaaagttggcgtttatatggggaaaaccaagtaatgttagtgaaagcaaagagatttggtgtagaatgattggcttgcttaggagtgaagtaggaattcatggaactgaagaaccgtctcaacttctcagaatcgttcctaacaactatagtttgcgccattgtctgtctctttcgggttaa

SEQ ID NO: 18 - protein, 372 aa, Arabidopsis thaliana
MSNADEPPQKTNQPPSSSLTPPSLFSLPVDIVLNILALVPKRYYPILCCVSKSLRSLIRSPEIHKTRSLHGKDSLYLCFSTRTTYPNRNRTTFHWFTLRRNDNKMNTTENVFVSIDVPYRPGHASYPLSNIAIDTEIYCIPGYNFPSSSIVWIFDTQSGQWRQGPSMQVERLSATVGLVGGKIYVIGGNRGEEILAEVFDLKTQTWEAAPIPKAKDRNEWFTHASVSLDRKVYALNSREYMNSYDTRDGSYQRYTIPEDNWWKTGKCVIDNVLFVYFLRFGLMWYDSELMLWRVVYGLDLDKARCIGIGEYYGKLAFIWGKPSNVSESKEIWCRMIGLLRSEVGIHGTEEPSQLLRIVPNNYSLRHCLSLSG

SEQ ID NO: 19 - DNA, 858 bp, Arabidopsis thaliana
  CDS (1)..(858)
atgttcggtgcacaatctagtgattcgttgccagagatttcacggccgactgcaacagaatgtcaagaggtgaaatccgatgaagcactcactgaacccaaccctgattgggctctagttctcgtcaaagacccatcgcctccactcatcatcgaggttgaggatgagactctgccttcaatcgacagtgttcagaaccctaaaccgaaggatcctgaggctaccccctcgcaaccctctgttgcatctcgattgcgcaagaggaagtcatctgctgcggatccacgcatcaaaaggatgaagcagggaaagggagttaccggttcatcttccttcgatggaattcgatttgtctctacggaggctgaaaagaggtatgctcaattctctcaacgaaatttcattgaggaagtagaactgtctaggaagacaaatgaggcagctagggagtttataaagcaggcaggactgattcgaacggttactaagttcaacccgttcactcagaatctagtgtttgagttctgggctaatctgcccactatgaaggtagacacgtatatggtcaaagtcttggtgcgcaatcgggagtatgagctctcacctgggaagatcaacgagatgtatggtctcccttctgttgatgctagacagcagcggatggatatcgctggtctggttgatgaacaagtggctgaatttctcactggtgggaaagtcagtgttctgagcaagcttcaggtgagtgcctttacacctacgagtttggaactgtttaaactctgctgctcaaattcgtctcccacatccaacgctgggtatgctcagcctgatcgattgtatattacatttctttga

SEQ ID NO: 20 - protein, 285 aa, Arabidopsis thaliana
MFGAQSSDSLPEISRPTATECQEVKSDEALTEPNPDWALVLVKDPSPPLIIEVEDETLPSIDSVQNPKPKDPEATPSQPSVASRLRKRKSSAADPRIKRMKQGKGVTGSSSFDGIRFVSTEAEKRYAQFSQRNFIEEVELSRKTNEAAREFIKQAGLIRTVTKFNPFTQNLVFEFWANLPTMKVDTYMVKVLVRNREYELSPGKINEMYGLPSVDARQQRMDIAGLVDEQVAEFLTGGKVSVLSKLQVSAFTPTSLELFKLCCSNSSPTSNAGYAQPDRLYITFL

SEQ ID NO: 21 - DNA, 2766 bp, Arabidopsis thaliana
  CDS (1)..(2766)
atggttttgggtttaagctcaaagaacagaagatgctcatcggtacaagttgattacctgatccacattcatgacatcaagccttggcctccttctcagtcccttcgatctcttcgctctgttgttatccaatgggagaatggtgaccgcaattctggtaccacttctgttgttgctccttctcttggttctgtcattggcgaaggcaaaatcgagttcaacgaatcatttaagcttcccctcaccttgttgaaagatgtctctgctcggggcaaaggtggtgatgtcttcttcaagaacgtcttagagctcaacttatacgagcctcgcagagagaaaacacatcagcttttggctactgccaccattgatttggctgtgtacggtgttgtcaaagaaagtttcagcttgactgctcaaatgaacagtaaacgaagctaccggaatgccactcagcctgttctgtacctcactattcaacccgttagtagacgccgggccagttcttcatctatgaacagcttgaaggatgaagccaagaatgggggtgaatctgtttctgccttgatgaatgaggaatactataaggaagctgagattgcttccatcacagacgacgacatttcctcccactcctccttgacagtctcatcttcaactttagagtctaatggtggtttttctgtgcgaaccgaagaggaagaacatgagcgaataaacaagaatcccagagggaatggccacgagagatcaaaatcagtgtcagagagcagacaaagacaaattgctgaccagattccgtcacgttcctcgtcagtagatctatcttctgtgtttcatctcccggagggcatttctgattctgcacccaacacttctttgtctgggctggagcactgtgctaatgtgtttataactgataccaatgagagctcgaagctggcaagtaacggtcagcacaataatggagaggccaagtctgtgcctttacaaattgacaatctttcggaaaatgcttcgcctcgagcctctgtgaatagtcaagatttgaccagtgatcaggaaccagagagtatagttgagaaatcaagaaaggtgaagtctgttcgatcatccttggatattaaccggagcaatagccggttgagtttattcagtgaaagaaaagaggctaaggtgtatccaaattctacacacgacacaactttggagagcaaaatcaagaacttggaaagtagagtaaagaagcttgagggtgagttatgcgaggctgcagccattgaagcagctctctactccgtggttgcagagcatggaagttcatcaagtaaagtccatgcgcctgctagacgtcttttgaggctgtatcttcatgcttgtagagaaacccatctctcaagaagagccaatgcagctgaaagtgctgtctccgggttagttcttgttgccaaagcgtgtggaaacgatgtccccagattgacattttggttgtcaaacactatcgttctgaggacaatcattagtgatactagtgcagaagaggaactgcctgtttctgctggtcctggaccaagaaaacagaaggctgaaagagaaaccgagaaacgatcgtccttgaagtggaaagattcccctttgagcaaaaaggacattaagagttttggtgcctgggatgaccctgtaactttcataacagcgcttgaaaaggtcgaagcttggatcttctcacgcgtcgtagaatcaatttggtggcagactttgacaccacgtatgcagtcttcggcagcatcaactagagagtttgataaaggaaatggctctgcatcaaagaaaacttttggtaggactccgagtagtacgaaccaagaactaggtgacttctcattagagctttggaaaaaggctttcagggaggcacacgaacgtttatgccctctgcgaggtagtgggcatgaatgtggctgcttgcccatacctgcgcgtctgataatggaacagtgtgtggctcggctagatgttgctatgttcaatgctatccttcgtgattctgatgacaatttcccaacagaccctgtatctgatcccattgcagacttgagagtcttgccaattccttctagaacatcaagttttggttctggtgcccagctgaaaaactcgataggaaactggtcaaggtggctcactgatctattcggcattgatgacgaagatgatgacagcagcgatgagaatagctatgttgaaaaatcgttcaagacattcaatcttctcaaggcgttaagcgatctaatgatgcttccaaaagatatgctcctcaacagttctgtgagaaaagaggtttgcccgatgtttggtgccccgttgattaagagagtgttgaataattttgtcccagatgagttttgccctgacccagttcctgatgctgtactgaaatctcttgaatccgaggaagaagctgagaagagtatcatcacaagctatccatgcactgcaccttctccagtgtactgtccaccttcacggacttcaatctccaccatcattggaaactttggacagcctcaagcgccgcagttaagcagaatcaggtcttcaattacaagaaaagcgtacacaagcgatgacgagcttgatgagctgagttcaccgttggctgttgttgttcttcaacaagcaggttctaagaagatcaataatggtgatgcagatgagacaatcaggtatcaacttctcagggagtgttggatgaacggcgaatga

SEQ ID NO: 22 - protein, 921 aa, Arabidopsis thaliana
MVLGLSSKNRRCSSVQVDYLIHIHDIKPWPPSQSLRSLRSVVIQWENGDRNSGTTSVVAPSLGSVIGEGKIEFNESFKLPLTLLKDVSARGKGGDVFFKNVLELNLYEPRREKTHQLLATATIDLAVYGVVKESFSLTAQMNSKRSYRNATQPVLYLTIQPVSRRRASSSSMNSLKDEAKNGGESVSALMNEEYYKEAEIASITDDDISSHSSLTVSSSTLESNGGFSVRTEEEEHERINKNPRGNGHERSKSVSESRQRQIADQIPSRSSSVDLSSVFHLPEGISDSAPNTSLSGLEHCANVFITDTNESSKLASNGQHNNGEAKSVPLQIDNLSENASPRASVNSQDLTSDQEPESIVEKSRKVKSVRSSLDINRSNSRLSLFSERKEAKVYPNSTHDTTLESKIKNLESRVKKLEGELCEAAAIEAALYSVVAEHGSSSSKVHAPARRLLRLYLHACRETHLSRRANAAESAVSGLVLVAKACGNDVPRLTFWLSNTIVLRTIISDTSAEEELPVSAGPGPRKQKAERETEKRSSLKWKDSPLSKKDIKSFGAWDDPVTFITALEKVEAWIFSRVVESIWWQTLTPRMQSSAASTREFDKGNGSASKKTFGRTPSSTNQELGDFSLELWKKAFREAHERLCPLRGSGHECGCLPIPARLIMEQCVARLDVAMFNAILRDSDDNFPTDPVSDPIADLRVLPIPSRTSSFGSGAQLKNSIGNWSRWLTDLFGIDDEDDDSSDENSYVEKSFKTFNLLKALSDLMMLPKDMLLNSSVRKEVCPMFGAPLIKRVLNNFVPDEFCPDPVPDAVLKSLESEEEAEKSIITSYPCTAPSPVYCPPSRTSISTIIGNFGQPQAPQLSRIRSSITRKAYTSDDELDELSSPLAVVVLQQAGSKKINNGDADETIRYQLLRECWMNGE

SEQ ID NO: 23 - DNA, 609 bp, Arabidopsis thaliana
  CDS (1)..(609)
atgaatcctgaatatgactatctgttcaagcttctgcttattggtgattctggtgttggaaaatcatgcttgcttctaagatttgctgatgattcttacctggatagctacataagcaccattggtgttgactttaaaattcgcacagttgagcaggacggaaagaccatcaaactccagatctgggacacagcaggccaagaacgtttcaggacaatcactagcagctactacagaggagctcatgggatcattgtcacttatgatgtcacagacctagagagcttcaacaacgtcaaacaatggctgaatgaaattgaccgttacgctagcgaaaatgtgaacaagctactggttgggaacaagaacgatctcacttcacagaaagttgtatccactgagacagctaaggcttttgcagatgaacttgggatcccattcttggaaacaagtgctaaaaatgcaaccaatgtggaagaagctttcatggctatgactgctgcaattaagacaagaatggctagccaaccagctggaggtgccaagccaccaacggtccagatccgtggacagccagtgaaccagcaatcaggctgttgttcttcttga

SEQ ID NO: 24 - protein, 202 aa, Arabidopsis thaliana
MNPEYDYLFKLLLIGDSGVGKSCLLLRFADDSYLDSYISTIGVDFKIRTVEQDGKTIKLQIWDTAGQERFRTITSSYYRGAHGIIVTYDVTDLESFNNVKQWLNEIDRYASENVNKLLVGNKNDLTSQKVVSTETAKAFADELGIPFLETSAKNATNVEEAFMAMTAAIKTRMASQPAGGAKPPTVQIRGQPVNQQSGCCSS

SEQ ID NO: 25 - DNA, 990 bp, Arabidopsis thaliana
  CDS (1)..(990)
atggagacaccatcatcgacaagaagagtcacgagatctcagacccgctgtgctatcaataatctcttatcctcaaagaaagcggatgattggtcagataaatgtcaatcgaagtcggtacaaagaaacggtgcaaaagacaggtctgcgttgtttgacataaccaacgactcaccgatcgtaggtctggcgatgcagacaccgtcctcaggagttgttgggaagaggaacatgagcagaatcaataacactcctggatctggtgaggctctcttgagaggccaagtcaagactctcttgcagaaggttgaggaagaagcagatctcaccaagatttcgttggaatctcgtcctttcatccatctcgttacttctcccatgggtcttctcgccccaactccggccaacaccccccaagttcttggtttctcagacgaagtgcaggttgtgatcacttctccggtagttgcaggacaattcagagcaccgtctcaggtggtggaaagcaacatgtttgatgagaaagaagaaagcttggagttagagaagagcccgagcattactcggtcattgcttctggactttagtgataaatcagaattatgggagagctcagactgctcatctgtggtgactcaaaacccagaggatgataactcgtcggtgtggtcgatgcaggccaatgcaagtgccaaagacgaagagtatgatgatgaagaggaagaagcctattcttacggtgaggaatatgatgaggaatactacgacgaggaagaggaggaagaagagggagggattgtggatgggttgtgtgagggaattaggaagatgagcgtagagactgactttgcagggaaacacactagatttgtgtatgacagtgaagatgaagagattgtggaggccaaagatcaatctccaggagtgttgcgtttgaaaggctttcctacaccgacaggaaaacatgtcagatttgccggtgatgaatga

SEQ ID NO: 26 - protein, 329 aa, Arabidopsis thaliana
METPSSTRRVTRSQTRCAINNLLSSKKADDWSDKCQSKSVQRNGAKDRSALFDITNDSPIVGLAMQTPSSGVVGKRNMSRINNTPGSGEALLRGQVKTLLQKVEEEADLTKISLESRPFIHLVTSPMGLLAPTPANTPQVLGFSDEVQVVITSPVVAGQFRAPSQVVESNMFDEKEESLELEKSPSITRSLLLDFSDKSELWESSDCSSVVTQNPEDDNSSVWSMQANASAKDEEYDDEEEEAYSYGEEYDEEYYDEEEEEEEGGIVDGLCEGIRKMSVETDFAGKHTRFVYDSEDEEIVEAKDQSPGVLRLKGFPTPTGKHVRFAGDE

SEQ ID NO: 27 - DNA, 588 bp, Arabidopsis thaliana
  CDS (1)..(588)
atgaacaaaacccgccttcgtgctctctccccaccttccggtatgcaacaccgtaagagatgtcgattgagaggtcgaaactacgtaaggccagaagttaaacaacgcaacttctcaaaagatgaagacgatctcatcctcaagcttcatgcacttcttggcaatagatggtcattgatagcgggaagattgccaggacgaaccgacaacgaagttaggatccattgggaaacttacctaaaaaggaagctcgtaaaaatgggaatcgacccaaccaatcatcgtctccaccatcacaccaactacatttctagacgtcacctccattcttcacataaggaacatgaaaccaagattattagtgatcaatcttcttcggtatccgaatcatgtggtgtaacaattttgcccattccaagtaccaattgctcggaggatagtactagtaccggacgaagtcatttgcctgacctaaacattggtctcatcccggccgtgacttctttgccagctctttgccttcaggactctagcgaatcctctaccaatggttcaacaggtcaagaaacgcttcttctattccgatga

SEQ ID NO: 28 - protein, 195 aa, Arabidopsis thaliana
MNKTRLRALSPPSGMQHRKRCRLRGRNYVRPEVKQRNFSKDEDDLILKLHALLGNRWSLIAGRLPGRTDNEVRIHWETYLKRKLVKMGIDPTNHRLHHHTNYISRRHLHSSHKEHETKIISDQSSSVSESCGVTILPIPSTNCSEDSTSTGRSHLPDLNIGLIPAVTSLPALCLQDSSESSTNGSTGQETLLLFR

SEQ ID NO: 29 - DNA, 864 bp, Arabidopsis thaliana
  CDS (1)..(864)
atgtcgtggtcgaaagcttgtagaggaactcgaatttcttcatatttagagaaccttcatcgaacatctcagtatccaaggactattttatgctctcgttattacactcatggagcttgtaaaagcaatgagcattatctccgaagcaagagagtattttggggaagctcttcttcatggagcttgaattctcactctgctactgctaaatcgatgttagatagtgcccatcgccagtattctactcactctccatcggaaaccaaatcacagaaaatgctctattacctcaccgctgttgtctttggtatggtggggttaacttacgctgctgtgccattgtatagaacattctgccaagctactggatatggaggtactgttcaacgcaaagagactgttgaggagaagattgctaggcattcagaatctggtaccgtcactgaaagggagattgtggtgcagttcaatgctgatgttgcagatgggatgcagtggaagttcactccaacacaaagagaggttagagtaaagccaggagaaagtgcactcgcattttacactgctgaaaacaaaagttcagctccaataaccggagtctcgacatacaatgtcactcccatgaaggcaggagtttatttcaacaagatacaatgtttttgctttgaggagcagcgactccttcctggagagcagattgacatgccggtcttcttctacattgatcctgagtttgagactgatccaagaatggacggaatcaacaacttgatattgtcttacactttcttcaaagtgtcagaggaaaatactacagagacggtcaacaataacaactctgttccagttcaagaaaccaattaa

SEQ ID NO: 30 - protein, 287 aa, Arabidopsis thaliana
MSWSKACRGTRISSYLENLHRTSQYPRTILCSRYYTHGACKSNEHYLRSKRVFWGSSSSWSLNSHSATAKSMLDSAHRQYSTHSPSETKSQKMLYYLTAVVFGMVGLTYAAVPLYRTFCQATGYGGTVQRKETVEEKIARHSESGTVTEREIVVQFNADVADGMQWKFTPTQREVRVKPGESALAFYTAENKSSAPITGVSTYNVTPMKAGVYFNKIQCFCFEEQRLLPGEQIDMPVFFYIDPEFETDPRMDGINNLILSYTFFKVSEENTTETVNNNNSVPVQETN

SEQ ID NO: 31 - DNA, 1056 bp, Arabidopsis thaliana
  CDS (1)..(1056)
atgatttctgaagaccttttggtcgaaattctcttaagattgcctgtgaaacccctagctagatgcctatgtgtgtgcaagctttgggccacaatcatccgcagtcgatatttcatcaacttataccaatctcgatcctctactcgtcagccatatgtcatgtttgctttacgtgatatattcacatcttgtcgttggcatttcttctcgtcatcccaaccttctttggttaccaaggcgacatgcagcgccaataattcttctcatacgcctgattgtgtcaatggcttgatatgtgtcgaatacatgtctcaactgtggatatccaatcctgccacgcgtaagggtgtactcgtgccccaatccgctccacatcaaaagttcaggaaatggtatatgggatatgatcctatcaattatcaatacaaggttttgtttttctcaaaacaatatttattatctccctataaactggaagtgtttacattggaaggtcaaggttcatggaaaatgatcgaggttgagaatattccttctccctcaaccagaggaatatgcatcgatggggttgtgtattacggtgctcagacggcgcatggactgaggcttgttagattctacgtggctactgaaaagtttggtgatttcattgaaataccggtcggggctagtaatgtttatgatatgaactttggttactctaaacttgtgaactatcaagggaaactagctttacttgctgcaaaaagtatgagcatgtatgatttatgggttttggaagatgccgggaaacaagaatggtctaaggtttctatcgttttaactcgagagatgttttcgtatgatcttgtttggctaggtgcggtgggttttgttgctggtagtgatgagcttatagttacagctcatgatcgattttatcagatttatcttgtctatgttgatctcaagatgaaacgttcaagggaagtttggctcggagggatcagatgctcagatcgatcatctttagtgcttactttcacggattacgttgaaagtattatgttattgtaa

SEQ ID NO: 32 - protein, 351 aa, Arabidopsis thaliana
MISEDLLVEILLRLPVKPLARCLCVCKLWATIIRSRYFINLYQSRSSTRQPYVMFALRDIFTSCRWHFFSSSQPSLVTKATCSANNSSHTPDCVNGLICVEYMSQLWISNPATRKGVLVPQSAPHQKFRKWYMGYDPINYQYKVLFFSKQYLLSPYKLEVFTLEGQGSWKMIEVENIPSPSTRGICIDGVVYYGAQTAHGLRLVRFYVATEKFGDFIEIPVGASNVYDMNFGYSKLVNYQGKLALLAAKSMSMYDLWVLEDAGKQEWSKVSIVLTREMFSYDLVWLGAVGFVAGSDELIVTAHDRFYQIYLVYVDLKMKRSREVWLGGIRCSDRSSLVLTFTDYVESIMLL

SEQ ID NO: 33 - DNA, 10725 bp, Arabidopsis thaliana
  CDS (1)..(10725)
atgacgtcttcttcgcataatattgagttggaagcagctaagtttctgcataaacttattcaagattctaaagatgaacctgcaaagctagcaactaaactttatgtgatattgcagcacatgaaaaccagtggaaaggaaaacacaatgccctatcaagtcatatctagggccatggacactgtagtcaatcaacacggtcttgacattgaagctttgaagtcctcgtgtcttcctcatcctggtggtacccaaacggaagattctgggtctgcacatttggctgggtcttcccaagcggttggagttagcaatgagggtaaagcaactctagttgaaaatgagatgacaaaatacgatgcattcacttctggtaggcagcttggtggatcaaacagtgcgtcacaaaccttttaccaagggtctggaactcaaagtaacagatcatttgatcgtgagagtccatccaacttggattctacgtctggaatttctcaacctcacaatcgtagtgagacaatgaatcaaagagatgtcaagtcgagtggaaagagaaagagaggtgaatcatcactttcatgggaccagaatatggataactctcaaatatttgatagccacaagattgatgatcaaactggagaagtaagcaaaatagaaatgcccggtaattcaggtgacattaggaatttgcatgttgggttgtcctcagatgcattcacgactccacagtgtggctggcagagcagtgaagccacagcaatcaggcctgcaattcataaagaacctggaaataatgttgcaggagagggattcctgccttcaggttcaccttttagagagcaacaactgaagcagctcagagcccagtgccttgtgtttctagctcttagaaatggtttggtaccgaagaaacttcacgtagagattgcccttcgaaatactttccgcgaagaagatggtttccgcggagaattgtttgatccgaaagggagaacacatacgtccagtgatctgggtggcattcctgacgtctctgcactgttgtcaagaacagacaatcccactggaagattggatgaaatggacttctcatccaaagaaactgagagatcaagactaggggaaaaaagtttcgcaaatactgtattttctgacgggcaaaagcttctagcatctagaattcccagctctcaagcacaaactcaggttgctgttagtcattctcaactaacgttttctcctggtttgaccaaaaacacaccatcagagatggtggggtggactggagtaatcaaaactaatgacctctcaacttctgctgttcaactcgacgaatttcattcttctgatgaagaggaaggtaatttgcaaccgtcgcctaagtacaccatgtcacagaaatggattatgggtcgacagaataagagactattggttgatcgtagttggtcccttaaacagcagaaagccgaccaggcaattggttcacggttcaatgagttgaaggaatctgtgagtttgtctgatgatatatctgcaaagaccaagagtgtaatagaactgaaaaagcttcagctgttaaatctacaacggcgattgaggagtgagttcgtttacaacttcttcaaacctattgcaactgatgttgagcatctaaagtcatataagaaacataagcatggccggagaattaagcagcttgaaaagtatgagcagaagatgaaggaagagagacaacgaagaattcgggagagacagaaggagttctttggagggttagaagttcacaaggaaaaactagaggatttgtttaaagttaggagagaaaggttgaagggtttcaatagatatgcaaaggagttccacaaaagaaaggaacgacttcaccgtgagaagattgacaaaattcaacgtgagaagattaatttgttaaagataaatgatgtggagggttatctccgaatggtgcaggatgccaagtcagatcgagtaaagcaactactcaaagagactgaaaagtaccttcagaaacttggatccaagttaaaagaggctaaattgttgaccagtcgatttgagaatgaggcagatgaaacgcgtacgtcaaatgctaccgacgatgaaactttgattgaaaatgaagatgagagcgaccaagcaaagcattacctggaaagcaacgaaaaatactacttgatggctcacagtataaaagaaaatattaacgagcagccatcgtccctagtgggtggaaagttaagagagtaccaaatgaacggcttaaggtggctagtttcactgtacaacaatcacttaaatggcattttagctgatgagatgggtctcgggaaaactgttcaggttatttctttgatttgctatctgatggagacaaaaaatgatagaggtcccttcttggttgttgtcccatcttcggttctgcctggttggcagtcagaaattaacttctgggccccatcaattcataaaattgtttactgtgggactcctgacgaaaggcgcaaactattcaaggagcaaattgttcatcagaagttcaatgtccttctgacgacatatgagtatctaatgaacaagcatgataggcctaaattaagcaagattcactggcactatattattattgacgaaggacatcgcataaagaatgcatcctgcaagttaaatgcagacctgaaacattatgttagctcccaccgacttctgttaactgggaccccattgcagaacaacctagaagaattgtgggcactgcttaatttcctgctgcctaatatattcaactcgtcagaagacttttcacagtggttcaacaaaccattccaaagtaatggagaaagttctgccgaggaggcattgctatcagaagaggagaatctactgatcatcaatcgacttcaccaagttcttcgaccatttgttctgcgtcggctgaaacataaggttgaaaatgagcttcccgaaaagatagagagattgatacgctgtgaggcttctgcttatcagaagctgttgatgaagagggttgaggataatttgggttcgattggaaatgcgaagtctcgtgcagtgcacaactcagtaatggagcttcggaatatttgcaatcatccatatcttagccaactgcattcagaggaggtcaataacataattcctaagcatttcctgcctccaattgtaagactgtgtggaaagcttgagatgttggatcggatgttgcctaaactcaaagcaacagaccatcgggttcttttcttttccacaatgacaaggcttcttgatgtaatggaggattacctcaccttaaaggggtacaaataccttaggctggacgggcagacatctgggggtgatcgtggtgctcttattgatggtttcaacaagtcgggttccccatttttcatatttctgttgagcattcgggctggtggagtaggagttaatcttcaagctgctgatactgtgatactattcgacactgactggaatcctcaggttgatctgcaagctcaagctagggctcacaggattggccagaaaaaagatgtgcttgttcttcgttttgaaacggtgaattctgttgaggagcaagtcagagcttcagctgagcacaaacttggagttgctaaccagagtataaccgctggcttttttgacaataacacgagtgctgaagatcgtaaggaatatttggaatcccttttgcgtgaatcaaagaaagaggaggatgctccagtattggacgatgatgccttaaatgatcttatagctcgaagggagtcagagattgatatctttgagtccatcgacaaacaaaggaaagaaaatgagatggaaacatggaataccttggtacatgggccagggtcagatagttttgcccacataccatctataccctctcggcttgttactgaagatgatttaaaactactatatgaaacaatgaaactaaatgatgtccccatggttgcaaaagaatcaactgttggcatgaagcggaaggatggttcgatgggaggcctagatactcaccaatatggaagagggaaacgagcaagagaggttcgatcttatgaagagaaattaactgaggaggagtttgagaagctgtgtcagactgagtcacctgattcaccccaaggcaagggcgaaggaagtgaaaggagcttggcgaatgatacatcaaatattccggttgaaaattctagtgacacactactccctacatctccgacacaggcaattactgtacaaccaatggaacctgtaaggccacagtcacatacactgaaagaagaaacacaacctataaaacgtggtcgtggtaggccaaagagaactgataaagccttgactccggtatcattatcagctgtaagcaggacacaggcgacagggaatgctatatcatcggcagcaactggtcttgattttgttagttcagataaaaggctagaagccgcttctcatcctacttcgtctcttgccctgacttcgcctgatttatctggtcctcctggttttcaatcattgccagcatctcctgctcctacgccaataagaggccgaggtaggggaagaagcagggggcgtggagcaggaaggggaagaagagttgaaggtgtattgcatggttccaacagttctattactcagagaactgaaaccgctacatctcttgcaagtgatgcagaagccacaaaatttgctcttcctcgttctgcatctgagattgtttcaagagtccctaaagctaatgaaggaagtacttcgaatcctgaccaagtatctcccgtgcattctgccactactgcactccgatcagacaaagcagcagataaggacctagatgcaccacctggttttgattcgggatcccatgttcagacgctaaatgtacttgaaaattcatcggaaagaaaagcatttgctgtaaagaagaggcctttgatccaaggtgtgagctcccagcatccagggcctaataaacagccacttgacttgccagtctccactagctccactttgttgggtggtggcccagtgcaaaatcagaatgccgtctcttctgtttgtgatggatccaaaagtccttctgagggtcgaacatatacagctttgcaaggtgtgacaactgcaccttctgatgctactctgccaatgagttctcaaccttctgatgctactctgccaatgagttctcaacctgtcggttctacggttgaagctcaagaagccaatgttccttctcttccagcagccttgcctgctaagaggcgagtccgcaatttgccaagcagaggagaaactcctaaacgccaaggaaagaggcgtggccaacctttacctgcaaccgatgcctcttctgcaaggagtacaggattaacaccacaaatagaggtcaaggttggtaatttatcaggcaccaaagctaagtttgatgctgttgccaaagaacaaccccacttcagccagtcagttgcacccgatattcactcttctggtagtttgagtcaggaaattagaagagacacctctggtactggtggttctgctaggaaacaaactgctgatgtaactgatgttgctcgagtcatgaaagagatcttttcagagacttccctattaaaacataaagttggagagccttctgcaacaacgagaacaaatgtgcctgacgcacaatcccctggtgagatgaatttgcacacagttgagacccacaaggcagaggattcttctggtcttaagaatcaagaagctttatataacctgagcaaggcagataaactggtatcagatattcctcatcctgttcctggtgatctgacaacttcaggatcagttgcaaacaaagatgttgacattgggtcgtctaaggttgctgctgaaaatgagcttgtcaaaattccgggtggtgacgtagattcttctgtaatacaactctctttgggaaatactttgactgctaaatcgtctttggaaaagtgcactgcagatcagcttctgggagaaaaactgtctcaagaaggtgaaaccacacctgctagtgatggtgaaacatgtcacctggcagaagaaacggcatcttcattgagttatgttcgatctgagcctactgcatctgcgtcgacaactgcggaacctctacctactgacaagttggaaaaaaatatttcttttcaagatgaagttaaaactctcaatggtgataaaagagaagctatcctcctaagttcggaagagcaaacgaatgttaactccaagattgagacaaattctgaggaacttcaagccagtagaacagatgaagttccacatgtggatggaaaatctgttgatgttgcaaatcagacggtgaaagaagatgaggcaaaacattctgttgaaattcaatcgtctatgctggagcctgatgaactgccaaatgctggacaaaagggtcacagtagcattgacttgcagccattggttttagttacaagcaatgagaatgctatgtcccttgacgataaagattatgatcctatctctaaatctgctgatatagaacaagatcctgaagaatctgtttttgttcaaggtgttggtaggcctaaagttggtactgctgatacacagatggaggataccaatgatgccaaacttctagtgggttgttcagttgagagtgaggaaaaagagaaaactcttcaatccctcatacccggtgatgatgctgatacagaacaagatcctgaagaatctgtttcggatcaaaggcctaaagttggttctgcttacacacagatggaggatacggatgaggcgaaacttctaatgggttgttcagttgagagtgaggaaaaagagaaaactcttcaatcccatatacccggtgatgatgctgatacagaaaaaaatcctgaagaatctgtttccgttcaaggtgttgataggccgaaagttggtactactgacacacagatggaggataccaatgatgccaaacttctagtgggttgttcagttgcgagtgaggagaaagagaaaactcttcaatcccatatacccggtgatgatgctgatacagaacaaaatcctgaagaatctgtttcagttcaaggtgttaataggcctaaagttggtaatgctaacacacagatggaggatacggatgaggccaaagttctagtgggttgttcagttgagagtgaggagaaagagaaaactcttcaatcccacatacctggtgatgatgctgatacagaacaaaatcctgaagaatctgtttcgaattttgataggcctaaagatgggactgctgacacacatatggaggatatcgatgatgccaaacttctagtgggttgttcagttgagagtgaggagaaagagaaaagtcttcaatcccatatgcccagtgatgatgctgttctccatgcgccttttgagaacacaaaagacagtaaaggagatgatttacatggagagtctcttgtttcctgtccaacaatggaagtgatggaacagaaggggtttgaatcagagacacatgctcgtacagattcaggtggtattgataggggaaatgaggtatcagaaaatatgtctgatggcgtcaaaatgaatatttcatctgtgcaggtcccggatgcatcacatgatttaaatgtatcacaggatcaaacagacattcccctagttggtgggatagaccctgaacacgtacaagagaatgtggatgtacctgcatcacctcacggagcagcgccaaacattgtgattttccagtctgagggacatctgtctccaagtatcttaccggacgatgtggcaggacaactagaaagcatgtctaatgacgaaaaaacgaatatttcatctgagcaggtcccagatgtatcacatgatttgaaagtgtctcaggatcaaactgacattcccccagttggtgggatagtgcctgaaaatttgcaagagattgtggatgtacctgcatcacctcatggagtagtgccagacgttgttgtttcccagtctgaggaaattcaatctccaagtattttgcccgacgatgtaccaggacaaccagacgatggcaactgtgagaaaatggataccatgcagaacaatacctctattgatattggcataacttcaggtaagacatgtcagccttcatcttctacccagcctgaggatgagaacagaaatagcttatcacactgtgaaccgtcagaagtagttgaacaaagggattcaagagatcaagtttgcatagggtctgtggaatctcaagtagagatcagctctgctatactggaaaatagatcagctgatatccagcccccgcaatccattttggttgatcaaaaggatattgaagaatccaaagaacctggtatcgagagtgctgatgtgtctttacaccaattagctgatatccaggccgagccatccaatttggttgatcaaatggatattgaagaatccaaagaacctggtaccgagagtgctgatgtgtctttacaccaattagctgatatccagcccgggccatccattttggttgatcaaatggatactgaaaaatccaaagaacctggtaccgagagtgctgatgtgtctttacaccaattagctgatatccagcccgggccatccattttggttgatcaaatggatactgaaaaatccaaagaacctggtaccgagagtgctgatgtgtctttacaccaattagctgatatccagcccgggccatccattttggttgatcaaatggatactgaagaattcaaaaatcctgatgtgtctttacaccaattagctgatattgagccctcactgtctatttcagctgtgcaaaagaatattgaggataaggatcaaagtcacgttgaaactgctggatctgagttagttgatgtctctgccgaatgttcaacagaacctcaagttcaattaccgccatcttcagagccagtgggagatatgcacgttcatttaggggcaagcaaatcagaaatagttgccgaaggtactgacttctcttcatctctcccgaagacggaggaagaaaatgccaagagccaattagctgacaccgagccatcatcgtctcttacagctgtgcaaaagaacattgaagatcaagttgaaactgctggatgtgaatttgttgttgtctctaccggatgttcaacagaaccacaagttcaattaccgccgtccgcagagccagtggttgctgaaggtacagaattcccttcttccctcctaatgaccggggtagataattcttcccatctaatgaccggggtagataatgccaagacccatctcgctgatgttgtgccttcatcgtcacctacaactatggaaaagaacattgaagctcaagatcaagatcaagttacaactggtggatgtggtctagttgatgtcttgaccgaatgttcgtcagaacctcaacttcaactgccgccatccgcagaaccagtgatttctgaaggtacagaactcgctacactcccattgacggaggaagaaaatgctgatagccaattagctaatattgagccctcatcgtctccttcagttgtggaaaagaacattgaggctcaagatcaagatcaagttaaaactgctggatgtgagttagtctcgactggatgttcgtcagaaccacaagttcatttaccgccctccgcagagccagatggagatatacacgttcacttaaaggaaacagagaaatctgaaagcatggttgtggttggcgaaggtacagcattcccttcatctctcccagtgacagaggaaggaaatgctgagagccaattagctgacactgagccctttacgtctcctacagttgtggaaaagaacattaaggatcaagaacaagttgaaactactggatgtgggttagttgatgattctaccggatgttcgtcagaacctcaagttcaattaccgccatccgcagagccaatggaaggtacacacatgcacttagaggaaacaaagaaatctgaaactgtagttaccgagattcaattagctgatatagatccctcattttctcttatagttgtgcaaacgaatattgaggatcaagatcaaattgaaacaggtggatgtgatttaattaatgtcccttccggatgttcaacagaacctcaaattcaattatcgtcatccgcagagcccgaggaaggtatgcacattcacttagaggcagcaatgaactctgaaacggtggttactgaaggttcagaactcccttcatctctcccaatgacggaggacgaaaatgctgatggccaattagctgaagtcgagccctcagtgtctcttacagttgagcaaactaacattgaggagaaagatcacattgaaactgccgaatgtgagttagttgatgtctctcccggatgttcatcacaacctgaagttaaatttccgccatccccagatgcagtgggaggtatggacgttcacttagaaaccgttgttactgaagacacagattcaaattcatccctcccgaagacggaggaaaaagatgccgagaatccatcagacaggcttgacggtgaatccgatggtacaactgttgctactgttgaaggaacttgtgttgagtcgaattcattggtcgccgaagagagcaacatagaagtgccaaaagacaatgaagatgtgtag

SEQ ID NO: 34 - protein, 3574 aa, Arabidopsis thaliana
MTSSSHNIELEAAKFLHKLIQDSKDEPAKLATKLYVILQHMKTSGKENTMPYQVISRAMDTVVNQHGLDIEALKSSCLPHPGGTQTEDSGSAHLAGSSQAVGVSNEGKATLVENEMTKYDAFTSGRQLGGSNSASQTFYQGSGTQSNRSFDRESPSNLDSTSGISQPHNRSETMNQRDVKSSGKRKRGESSLSWDQNMDNSQIFDSHKIDDQTGEVSKIEMPGNSGDIRNLHVGLSSDAFTTPQCGWQSSEATAIRPAIHKEPGNNVAGEGFLPSGSPFREQQLKQLRAQCLVFLALRNGLVPKKLHVEIALRNTFREEDGFRGELFDPKGRTHTSSDLGGIPDVSALLSRTDNPTGRLDEMDFSSKETERSRLGEKSFANTVFSDGQKLLASRIPSSQAQTQVAVSHSQLTFSPGLTKNTPSEMVGWTGVIKTNDLSTSAVQLDEFHSSDEEEGNLQPSPKYTMSQKWIMGRQNKRLLVDRSWSLKQQKADQAIGSRFNELKESVSLSDDISAKTKSVIELKKLQLLNLQRRLRSEFVYNFFKPIATDVEHLKSYKKHKHGRRIKQLEKYEQKMKEERQRRIRERQKEFFGGLEVHKEKLEDLFKVRRERLKGFNRYAKEFHKRKERLHREKIDKIQREKINLLKINDVEGYLRMVQDAKSDRVKQLLKETEKYLQKLGSKLKEAKLLTSRFENEADETRTSNATDDETLIENEDESDQAKHYLESNEKYYLMAHSIKENINEQPSSLVGGKLREYQMNGLRWLVSLYNNHLNGILADEMGLGKTVQVISLICYLMETKNDRGPFLVVVPSSVLPGWQSEINFWAPSIHKIVYCGTPDERRKLFKEQIVHQKFNVLLTTYEYLMNKHDRPKLSKIHWHYIIIDEGHRIKNASCKLNADLKHYVSSHRLLLTGTPLQNNLEELWALLNFLLPNIFNSSEDFSQWFNKPFQSNGESSAEEALLSEEENLLIINRLHQVLRPFVLRRLKHKVENELPEKIERLIRCEASAYQKLLMKRVEDNLGSIGNAKSRAVHNSVMELRNICNHPYLSQLHSEEVNNIIPKHFLPPIVRLCGKLEMLDRMLPKLKATDHRVLFFSTMTRLLDVMEDYLTLKGYKYLRLDGQTSGGDRGALIDGFNKSGSPFFIFLLSIRAGGVGVNLQAADTVILFDTDWNPQVDLQAQARAHRIGQKKDVLVLRFETVNSVEEQVRASAEHKLGVANQSITAGFFDNNTSAEDRKEYLESLLRESKKEEDAPVLDDDALNDLIARRESEIDIFESIDKQRKENEMETWNTLVHGPGSDSFAHIPSIPSRLVTEDDLKLLYETMKLNDVPMVAKESTVGMKRKDGSMGGLDTHQYGRGKRAREVRSYEEKLTEEEFEKLCQTESPDSPQGKGEGSERSLANDTSNIPVENSSDTLLPTSPTQAITVQPMEPVRPQSHTLKEETQPIKRGRGRPKRTDKALTPVSLSAVSRTQATGNAISSAATGLDFVSSDKRLEAASHPTSSLALTSPDLSGPPGFQSLPASPAPTPIRGRGRGRSRGRGAGRGRRVEGVLHGSNSSITQRTETATSLASDAEATKFALPRSASEIVSRVPKANEGSTSNPDQVSPVHSATTALRSDKAADKDLDAPPGFDSGSHVQTLNVLENSSERKAFAVKKRPLIQGVSSQHPGPNKQPLDLPVSTSSTLLGGGPVQNQNAVSSVCDGSKSPSEGRTYTALQGVTTAPSDATLPMSSQPSDATLPMSSQPVGSTVEAQEANVPSLPAALPAKRRVRNLPSRGETPKRQGKRRGQPLPATDASSARSTGLTPQIEVKVGNLSGTKAKFDAVAKEQPHFSQSVAPDIHSSGSLSQEIRRDTSGTGGSARKQTADVTDVARVMKEIFSETSLLKHKVGEPSATTRTNVPDAQSPGEMNLHTVETHKAEDSSGLKNQEALYNLSKADKLVSDIPHPVPGDLTTSGSVANKDVDIGSSKVAAENELVKIPGGDVDSSVIQLSLGNTLTAKSSLEKCTADQLLGEKLSQEGETTPASDGETCHLAEETASSLSYVRSEPTASASTTAEPLPTDKLEKNISFQDEVKTLNGDKREAILLSSEEQTNVNSKIETNSEELQASRTDEVPHVDGKSVDVANQTVKEDEAKHSVEIQSSMLEPDELPNAGQKGHSSIDLQPLVLVTSNENAMSLDDKDYDPISKSADIEQDPEESVFVQGVGRPKVGTADTQMEDTNDAKLLVGCSVESEEKEKTLQSLIPGDDADTEQDPEESVSDQRPKVGSAYTQMEDTDEAKLLMGCSVESEEKEKTLQSHIPGDDADTEKNPEESVSVQGVDRPKVGTTDTQMEDTNDAKLLVGCSVASEEKEKTLQSHIPGDDADTEQNPEESVSVQGVNRPKVGNANTQMEDTDEAKVLVGCSVESEEKEKTLQSHIPGDDADTEQNPEESVSNFDRPKDGTADTHMEDIDDAKLLVGCSVESEEKEKSLQSHMPSDDAVLHAPFENTKDSKGDDLHGESLVSCPTMEVMEQKGFESETHARTDSGGIDRGNEVSENMSDGVKMNISSVQVPDASHDLNVSQDQTDIPLVGGIDPEHVQENVDVPASPHGAAPNIVIFQSEGHLSPSILPDDVAGQLESMSNDEKTNISSEQVPDVSHDLKVSQDQTDIPPVGGIVPENLQEIVDVPASPHGVVPDVVVSQSEEIQSPSILPDDVPGQPDDGNCEKMDTMQNNTSIDIGITSGKTCQPSSSTQPEDENRNSLSHCEPSEVVEQRDSRDQVCIGSVESQVEISSAILENRSADIQPPQSILVDQKDIEESKEPGIESADVSLHQLADIQAEPSNLVDQMDIEESKEPGTESADVSLHQLADIQPGPSILVDQMDTEKSKEPGTESADVSLHQLADIQPGPSILVDQMDTEKSKEPGTESADVSLHQLADIQPGPSILVDQMDTEEFKNPDVSLHQLADIEPSLSISAVQKNIEDKDQSHVETAGSELVDVSAECSTEPQVQLPPSSEPVGDMHVHLGASKSEIVAEGTDFSSSLPKTEEENAKSQLADTEPSSSLTAVQKNIEDQVETAGCEFVVVSTGCSTEPQVQLPPSAEPVVAEGTEFPSSLLMTGVDNSSHLMTGVDNAKTHLADVVPSSSPTTMEKNIEAQDQDQVTTGGCGLVDVLTECSSEPQLQLPPSAEPVISEGTELATLPLTEEENADSQLANIEPSSSPSVVEKNIEAQDQDQVKTAGCELVSTGCSSEPQVHLPPSAEPDGDIHVHLKETEKSESMVVVGEGTAFPSSLPVTEEGNAESQLADTEPFTSPTVVEKNIKDQEQVETTGCGLVDDSTGCSSEPQVQLPPSAEPMEGTHMHLEETKKSETVVTEIQLADIDPSFSLIVVQTNIEDQDQIETGGCDLINVPSGCSTEPQIQLSSSAEPEEGMHIHLEAAMNSETVVTEGSELPSSLPMTEDENADGQLAEVEPSVSLTVEQTNIEEKDHIETAECELVDVSPGCSSQPEVKFPPSPDAVGGMDVHLETVVTEDTDSNSSLPKTEEKDAENPSDRLDGESDGTTVATVEGTCVESNSLVAEESNIEVPKDNEDV

SEQ ID NO: 35 - DNA, 963 bp, Arabidopsis thaliana
  CDS (1)..(963)
atggctgctgataccacagcttcaagttactggttgaattggagggtcctcctctgtgcattaatcctcttagctccaatagtattagcagctgttctcatttggaaatatgaaggcaagagaagaagacaacgcgagagccagcgagaattaccagggacattgttccaagatgaagcttggaccacatgttttaaaagaatccaccctctttggttgcttgcctttagggtattctcgtttgttgcaatgttgactttactcatttccaatgttgtgcgcgatggagctggcatattctacttctatactcagtggacatttactcttgtcacactctactttgggtatgcttcggtgttatctgtttatggatgctgcatctataataaagaagctagtggaaacatggagagttatacaagtataggtgatacggaacaaggcacttatagaccaccaatcgctcttgatggggagggaaatacgtcaaaagcttccaatagaccttcagaagcacccgcccgaaaaacagcggggttttgggtttatatctttcagatccttttccaaacttgtgcaggtgctgttgtgctaacagatattgtattttgggcgataatctacccgtttactaaaggttacaagctgagttttctcgatgtttgtatgcattctctcaacgctgtttttctccttggtgacacaagcctcaattccttgcgattcccactgttccggattgcttactttgtactttggagctgtattttcgtggcttaccaatggataatccatgccgtcaagaacttatggtggccataccaatttcttgatctttcgtcaccatacgcacccttatggtacttgggagtggctgtgatgcatataccatgctttgcggtcttcgctttggtcataaagctgaagaattacttgctgcagcagcgtcacaactcgtga

SEQ ID NO: 36 - protein, 320 aa, Arabidopsis thaliana
MAADTTASSYWLNWRVLLCALILLAPIVLAAVLIWKYEGKRRRQRESQRELPGTLFQDEAWTTCFKRIHPLWLLAFRVFSFVAMLTLLISNVVRDGAGIFYFYTQWTFTLVTLYFGYASVLSVYGCCIYNKEASGNMESYTSIGDTEQGTYRPPIALDGEGNTSKASNRPSEAPARKTAGFWVYIFQILFQTCAGAVVLTDIVFWAIIYPFTKGYKLSFLDVCMHSLNAVFLLGDTSLNSLRFPLFRIAYFVLWSCIFVAYQWIIHAVKNLWWPYQFLDLSSPYAPLWYLGVAVMHIPCFAVFALVIKLKNYLLQQRHNS

SEQ ID NO: 37 - DNA, 4605 bp, Arabidopsis thaliana
  CDS (1)..(4605)
atggtggaaaatggggctaaagctgcgaagcgaaagaagagaccacttccagagattcaagaggtagaagatgtacctaggacgaggagaccaaggcgtgctgcagcgtgtaccagtttcaaggagaaatctattcgagtctgtgagaaatctgctactattgaagtaaagaaacagcagattgtggaggaagagtttctcgcgttacggttaacggctctggaaactgatgttgaagatcgtccaaccaggagactgaatgattttgttttgtttgattcagatggagttccacaacctctggagatgttggagattcatgacatattcgtttcaggtgctatcttaccttcagatgtgtgtactgataaggagaaagagaagggtgtgaggtgtacatcgtttggacgggttgagcattggagtatctctggttatgaagatggttcccctgttatttggatctcaacggaattggcggattatgattgtcgtaaacctgctgctagctacaggaaggtttatgattacttctatgagaaagctcgtgcttcagtggctgtgtataagaaattgtccaagtcatctggtggggatcctgatataggtcttgaggagttacttgcggcggttgtcagatcaatgagcagtggaagcaagtacttttctagtggtgcggcaatcatcgattttgttatatcccagggagattttatatataaccaactcgctggtttggatgagacagccaagaaacatgaatcaagctatgttgagattcctgttcttgtagctctcagagagaagagtagtaagattgacaagcctctgcagagggaaagaaacccatctaatggtgtgaggattaaagaagtttctcaagttgcggagagcgaggccttgacatctgatcaactggttgatggtactgatgatgacagaagatatgctatactcttacaagacgaagagaataggaaatctatgcaacagcccagaaaaaacagcagctcaggttctgcttcaaatatgttctacattaagataaatgaagatgagattgccaatgattatcctctcccatcgtactataagacctccgaagaagaaacagatgaacttatactttatgatgcttcctatgaggttcaatctgaacacctgcctcacaggatgcttcacaactgggctctttataactctgatttacgattcatatcactggaacttctaccgatgaaacaatgtgatgatattgatgtcaacatttttgggtcaggtgtggtgactgatgataatggaagttggatttctttaaacgatcctgacagcggttctcagtcacacgatcctgatgggatgtgcatattcctcagtcaaattaaagaatggatgattgagtttgggagcgatgatattatctccatttctatacgaacagatgtggcctggtaccgtcttgggaaaccatcaaaactttatgccccttggtggaaacctgttctgaaaacagcaagggttgggataagcattcttacttttcttagggtggaaagtagggttgctaggctttcatttgcagatgtcacaaaaagactgtctgggttacaggcgaatgataaagcttacatttcttctgaccccttggctgttgagagatatttggtcgtccatgggcaaattattttacagctttttgcagtttatccggacgacaatgtcaaaaggtgtccatttgttgttggtcttgcaagcaaattggaggataggcaccacacaaaatggatcatcaagaagaagaaaatttcgctgaaggaactgaatctgaatccaagggcaggcatggcaccagtagcatcgaagaggaaagctatgcaagcaacaacaactcgcctggtcaacagaatttggggagagttttactccaattactctccagaggatccattgcaggcgactgctgcagaaaatggggaggatgaggtggaagaggaaggcggaaatggggaggaagaggttgaagaggaaggtgaaaatggtctcacagaggacactgtaccagaacctgttgaggttcagaagcctcatactcctaagaaaatccgaggcagttctggaaaaagggaaataaaatgggatggtgagagtctaggaaaaacttctgctggcgagcctctctatcaacaagcccttgttggaggggaaatggtggctgtaggtggcgctgtcaccttggaagttgatgatccagatgaaatgccggccatctattttgtggagtacatgttcgaaagtacagatcactgcaaaatgttacatggtagattcttacaaagaggatctatgactgttctggggaatgctgctaacgagagggaactattcctgactaatgaatgcatgactacacagctcaaggacattaaaggagtagccagttttgagattcgatcaaggccatgggggcatcagtataggaaaaagaacatcactgcggataagcttgactgggctagagcattagaaagaaaagtaaaagatttgccaacagagtattactgcaaaagcttgtactcacctgagagagggggattctttagtcttccactaagtgatattggtcgcagttctgggttctgcacttcatgtaagataagggaggatgaagagaagaggtctacaattaaactaaatgtttcaaagacaggctttttcatcaatgggattgagtattctgttgaggattttgtctatgtcaaccctgactctattggtgggttgaaggagggtagtaaaacttcttttaagtctgggcgaaacattgggttaagagcgtatgttgtttgccaattgctggaaattgttccaaaggaatctagaaaggctgatttgggttcctttgatgttaaagtgagaaggttttataggcctgaggatgtttctgcagagaaggcctatgcttcagacatccaagaattgtatttcagccaggacacagttgttctccctccaggtgctctagagggaaaatgtgaagtaagaaagaaaagtgatatgcccttatcccgtgaatatccaatatcagaccatattttcttctgtgatcttttctttgacacctccaaaggttctctcaagcagctgcccgccaatatgaagccaaagttctctactattaaggacgacacacttttaagaaagaaaaagggaaagggagtagagagtgaaattgagtctgagattgtcaagcctgttgagccacctaaagagattcgtctggctactctagatatttttgctggttgtggtggcctgtctcatggactgaaaaaggcgggtgtatctgatgcaaagtgggcgattgagtatgaagagccagctgggcaggcttttaaacaaaaccatcctgagtcaacagtttttgttgacaactgcaatgtgattcttagggctataatggagaaaggtggagatcaagatgattgtgtctctactacagaggcaaatgaattagcagctaaactaactgaggagcagaagagtactctgccactgcctggtcaagtggacttcatcaatggtggacctccatgtcagggattttctggtatgaacaggttcaaccaaagctcttggagtaaagttcagtgtgaaatgatattagcattcttgtcctttgctgactatttccggccaaggtattttcttctggagaacgtgaggacctttgtgtcattcaataaagggcagacatttcagcttactttggcttcccttctcgaaatgggttaccaggtgagatttggaatcctggaggccggtgcatatggagtatcccaatctcgtaaacgagctttcatttgggctgctgcaccagaagaagttctccctgaatggcctgagccgatgcatgtctttggtgttccaaagttgaaaatctcactatctcaaggtttacattatgctgctgttcgtagtactgcacttggtgcccctttccgtccaatcaccgtgagagacacaattggtgatcttccatcagtagaaaacggagactctaggacaaacaaagagtataaagaggttgcagtctcgtggttccaaaaggagataagaggaaacacgattgctctcactgatcatatctgcaaggctatgaatgagcttaacctcattcgatgcaaattaatcccaactaggcctggggctgattggcatgacttgccaaagagaaaggttacgttatctgatgggcgcgtagaagaaatgattcctttttgtctcccaaacacagctgagcgccacaacggttggaagggactatatgggagattagattggcaaggaaactttccgacttccgtcacggatcctcagcccatgggtaaggttggaatgtgctttcatcctgaacagcacagaatccttacagtccgtgaatgcgcccgatctcaggggtttccggatagctacgagtttgcagggaacataaatcacaagcacaggcagattgggaatgcagtccctccaccattggcatttgctctaggtcgtaagctcaaagaagccctacatctcaagaagtctcctcaacaccaaccctag

SEQ ID NO: 38 - protein, 1534 aa, Arabidopsis thaliana
MVENGAKAAKRKKRPLPEIQEVEDVPRTRRPRRAAACTSFKEKSIRVCEKSATIEVKKQQIVEEEFLALRLTALETDVEDRPTRRLNDFVLFDSDGVPQPLEMLEIHDIFVSGAILPSDVCTDKEKEKGVRCTSFGRVEHWSISGYEDGSPVIWISTELADYDCRKPAASYRKVYDYFYEKARASVAVYKKLSKSSGGDPDIGLEELLAAVVRSMSSGSKYFSSGAAIIDFVISQGDFIYNQLAGLDETAKKHESSYVEIPVLVALREKSSKIDKPLQRERNPSNGVRIKEVSQVAESEALTSDQLVDGTDDDRRYAILLQDEENRKSMQQPRKNSSSGSASNMFYIKINEDEIANDYPLPSYYKTSEEETDELILYDASYEVQSEHLPHRMLHNWALYNSDLRFISLELLPMKQCDDIDVNIFGSGVVTDDNGSWISLNDPDSGSQSHDPDGMCIFLSQIKEWMIEFGSDDIISISIRTDVAWYRLGKPSKLYAPWWKPVLKTARVGISILTFLRVESRVARLSFADVTKRLSGLQANDKAYISSDPLAVERYLVVHGQIILQLFAVYPDDNVKRCPFVVGLASKLEDRHHTKWIIKKKKISLKELNLNPRAGMAPVASKRKAMQATTTRLVNRIWGEFYSNYSPEDPLQATAAENGEDEVEEEGGNGEEEVEEEGENGLTEDTVPEPVEVQKPHTPKKIRGSSGKREIKWDGESLGKTSAGEPLYQQALVGGEMVAVGGAVTLEVDDPDEMPAIYFVEYMFESTDHCKMLHGRFLQRGSMTVLGNAANERELFLTNECMTTQLKDIKGVASFEIRSRPWGHQYRKKNITADKLDWARALERKVKDLPTEYYCKSLYSPERGGFFSLPLSDIGRSSGFCTSCKIREDEEKRSTIKLNVSKTGFFINGIEYSVEDFVYVNPDSIGGLKEGSKTSFKSGRNIGLRAYVVCQLLEIVPKESRKADLGSFDVKVRRFYRPEDVSAEKAYASDIQELYFSQDTVVLPPGALEGKCEVRKKSDMPLSREYPISDHIFFCDLFFDTSKGSLKQLPANMKPKFSTIKDDTLLRKKKGKGVESEIESEIVKPVEPPKEIRLATLDIFAGCGGLSHGLKKAGVSDAKWAIEYEEPAGQAFKQNHPESTVFVDNCNVILRAIMEKGGDQDDCVSTTEANELAAKLTEEQKSTLPLPGQVDFINGGPPCQGFSGMNRFNQSSWSKVQCEMILAFLSFADYFRPRYFLLENVRTFVSFNKGQTFQLTLASLLEMGYQVRFGILEAGAYGVSQSRKRAFIWAAAPEEVLPEWPEPMHVFGVPKLKISLSQGLHYAAVRSTALGAPFRPITVRDTIGDLPSVENGDSRTNKEYKEVAVSWFQKEIRGNTIALTDHICKAMNELNLIRCKLIPTRPGADWHDLPKRKVTLSDGRVEEMIPFCLPNTAERHNGWKGLYGRLDWQGNFPTSVTDPQPMGKVGMCFHPEQHRILTVRECARSQGFPDSYEFAGNINHKHRQIGNAVPPPLAFALGRKLKEALHLKKSPQHQP

SEQ ID NO: 39 - DNA, 2337 bp, Arabidopsis thaliana
  CDS (1)..(2337)
atgacatcgcctggatcgaagaaagttgttaccaccgacgacggatcggagaagaaagtttccggcaatctcggaaaagtaagtttttccggagatcttaatcatagtggaagccatagtggaagccatagtcgtagcagcagcagcgccggaggtggtgaaggaggaacgtttgagtatttcggatgggtttatcatttgggtgtgaataagatcggtcatgagtactgtaatcttcgtttccttttcattagaggcaaatatgttgagatgtataaaagagatcctcatgagaacccagacattaaacctataaggcgtggtgtcatagggccgacaatggtgattgaagagctaggacgtcgaaaagttaatcatggggatgtctacgttataaggttctataaccggttggacgagtcaaggaaaggagaaattgcttgtgctactgctggagaagccctgaaatgggttgaggcttttgaggaagcaaagcagcaggctgaatatgcactatcgagaggcggaagtacaagaacaaagttaagcatggaggccaacatcgatctggaaggacatcggcctagagtacggcgttatgcttatggattaaaaaagcttatccgaattgggcaaggtccagagtctcttttgaggcaatcatctaccctggttaatgatgtcaggggtgatggtttctatgagggtggtgacaatggagatgcaatcgaggcacacgaatggaaatgtgttcgtacaatcaatggtgttaggatttttgaagatgttgctaacttcaaggctggtcgaggagttcttgtgaaagccgttgctgtagttgaagcaagtgcggataccgtgttcgaagtacttttgaacattgacaaacatcaaagatacgagtgggatgcagtaacaggtgattcggaaaagatagactcatacgagggtcactatgatgtcatttattgcatatacgatcctaaatatctttcgcgatggcagtcaaagagagattttgtgttctctagacagtgggttcgtgggcaagatgggacatatacaatcttacagtttccagcagtgcataagaaacggcctgcaaagtctggatatcgacgtacagaaatcacgccatcaacttgggaaatcaaaagcttaaaaaagcgcagcgatgcagaaactccaagttgtcttgtaacacacatgctagaaatccactctaaacgttggtgcaaatggaagaggaccagctactcaaagtttgaaaagactattccatatgcattactcctccaagtagcaggtctgaaggagtacattggagcaaacccagccttcaagtatgaaacttccgccacggttgtccagtccaagttccaagatgtccccaatggtgaatatgtagatgaagaaatggaggagcagttttatgatgccacggactcatcatctggagaagaggatgaggaggaaagcgatgacgatgatgaaaatcaggacaataaggagataaaagttaaactgaagaatgtttcatgggcaatcgctagcttatctttgaagcgacctaaagctccaggcgcaagtaatgtattagacgctagtgttgatcctgtgagtatcgatccaagtcagttccaaggatctctgcgcaaaggaaatggtgataaggactcaaattgctggaattctcctagtggcatggggtttatgatcaggggaaagacctacctaaaagataatgcaaaggtgatgggtgggcagccccttctaacacttatatcagtggattggttcaaagttgatagtgctgtagacaacatcgccctgcatccaaaatgtcttatccagtcggaacctgggaaaaagctcccattcatcctagtcatcaatctacaggtaccagcaaagcctaactattgtctggtactctactatgctgcggatcgacctgtaaacaagacttcatccctgggaaaattcgtggatgggagtgacagttaccgcgacgcaagatttaagctaattccaagtattgttcagggatattggatggtcaagcgggctgttggaacaaaagcctgcttgctcggtaaagctgtgacctgtaagtacctgaggcaggataactttttggagattgatgtagatattggatcatcggctgttgcgaggagcgttattggtctggtccttggttatgtcacaagcctcattgttgatctcgccatcttgattgagggaaaggaggaaagcgatttgcccgagtatatccttgggacagttcgacttaaccgaatagagctggactcagccgtatcatttgaggaatga

SEQ ID NO: 40 - protein, 778 aa, Arabidopsis thaliana
MTSPGSKKVVTTDDGSEKKVSGNLGKVSFSGDLNHSGSHSGSHSRSSSSAGGGEGGTFEYFGWVYHLGVNKIGHEYCNLRFLFIRGKYVEMYKRDPHENPDIKPIRRGVIGPTMVIEELGRRKVNHGDVYVIRFYNRLDESRKGEIACATAGEALKWVEAFEEAKQQAEYALSRGGSTRTKLSMEANIDLEGHRPRVRRYAYGLKKLIRIGQGPESLLRQSSTLVNDVRGDGFYEGGDNGDAIEAHEWKCVRTINGVRIFEDVANFKAGRGVLVKAVAVVEASADTVFEVLLNIDKHQRYEWDAVTGDSEKIDSYEGHYDVIYCIYDPKYLSRWQSKRDFVFSRQWVRGQDGTYTILQFPAVHKKRPAKSGYRRTEITPSTWEIKSLKKRSDAETPSCLVTHMLEIHSKRWCKWKRTSYSKFEKTIPYALLLQVAGLKEYIGANPAFKYETSATVVQSKFQDVPNGEYVDEEMEEQFYDATDSSSGEEDEEESDDDDENQDNKEIKVKLKNVSWAIASLSLKRPKAPGASNVLDASVDPVSIDPSQFQGSLRKGNGDKDSNCWNSPSGMGFMIRGKTYLKDNAKVMGGQPLLTLISVDWFKVDSAVDNIALHPKCLIQSEPGKKLPFILVINLQVPAKPNYCLVLYYAADRPVNKTSSLGKFVDGSDSYRDARFKLIPSIVQGYWMVKRAVGTKACLLGKAVTCKYLRQDNFLEIDVDIGSSAVARSVIGLVLGYVTSLIVDLAILIEGKEESDLPEYILGTVRLNRIELDSAVSFEE

SEQ ID NO: 41 - DNA, 708 bp, Arabidopsis thaliana
  CDS (1)..(708)
atggcggtagaagacactcccaaatctgttgtaacggaagaagctaagcctaattcaatagagaatccgattgatcgataccatgaggaaggtgatgatgccgaagaaggagagatcgccggaggagaaggagacggaaacgttgacgaatcgagcaaatccggtgttcctgaatcgcatcctctggaacattcatggactttctggttcgataatcctgctgtgaaatcgaaacaaacctcttggggaagttccttgcgacccgtgtttacgttttcaactgttgaggaattttggagtttgtacaacaacatgaagcatccgagcaagttagctcacggagctgacttctactgtttcaaacacatcattgaacctaagtgggaggatcctatttgtgctaatggaggaaaatggactatgactttccctaaggagaagtctgataagagctggctctacactttgcttgcattgattggagagcagtttgatcatggagatgaaatatgtggagcagttgtcaacattagaggaaagcaagaaaggatatctatttggactaaaaatgcttcaaacgaagctgctcaggtgagcattggaaaacaatggaaggagtttctcgattacaacaacagcataggtttcatcatccatgaggatgcgaagaagctcgacaggaatgcaaagaacgcttacaccgcttga

SEQ ID NO: 42 - protein, 235 aa, Arabidopsis thaliana
MAVEDTPKSVVTEEAKPNSIENPIDRYHEEGDDAEEGEIAGGEGDGNVDESSKSGVPESHPLEHSWTFWFDNPAVKSKQTSWGSSLRPVFTFSTVEEFWSLYNNMKHPSKLAHGADFYCFKHIIEPKWEDPICANGGKWTMTFPKEKSDKSWLYTLLALIGEQFDHGDEICGAVVNIRGKQERISIWTKNASNEAAQVSIGKQWKEFLDYNNSIGFIIHEDAKKLDRNAKNAYTA